(19) United States
(12) Patent Application Publication
(10) Pub. No.: US 2012/0189638 A1
(43) Pub. Date: Jul. 26, 2012

(54) METHOD OF TREATING NEURODEGENERATIVE DISEASE

(75) Inventors: Andrew Dillin, San Diego, CA (US); Ehud Cohen, Har Adar (IL)

(73) Assignee: Salk Institute for Biological Studies

(21) Appl. No.: 13/166,886

(22) Filed: Jun. 23, 2011

Related U.S. Application Data
(63) Continuation of application No. PCT/US2009/069410, filed on Dec. 23, 2009.
(60) Provisional application No. 61/140,469, filed on Dec. 23, 2008.

(51) Int. Cl. A61K 39/395; A61K 38/50; A61K 31/7088; A61K 38/45; A61K 38/02; A61P 25/28

(52) U.S. Cl. 424/158.1; 514/8.6; 514/44 A; 424/94.5; 514/17.7; 424/94.6; 514/17.8

(57) ABSTRACT

The invention is directed to a method of treating a patient suffering from Alzheimer's disease comprising administering to said patient an agent that reduces the activity of the IGF-1 signaling pathway.

### RELATED APPLICATIONS

This application is a continuation of International Application No. PCT/US09/69410, which designated the United States and was filed on Dec. 23, 2009, published in English, which claims the benefit of U.S. Provisional Application No. 61/140,469, filed Dec. 23, 2008. The entire teachings of the above applications are incorporated herein by reference.

### GOVERNMENT SUPPORT

This invention was made with government support under grant P01 AG031097 awarded by the National Institute of Aging. The Government has certain rights in the invention.

### BACKGROUND OF THE INVENTION

Alzheimer's disease (AD) is the most common neurodegenerative disorder associated with aging. The symptoms of AD include deterioration of cognitive function, memory impairment and personality changes. The deposition of amyloid beta (Aβ) is considered a hallmark of Alzheimer's disease. Aβ originates from the proteolysis of the Amyloid Precursor Protein (APP). The serine protease Beta Amyloid Cleaving Enzyme (BACE) cleaves APP which is then followed by intra-membrane cleavage of the resulting fragment by presinilin1. These events release a subset of aggregation prone peptides Aβ, including Aβ1-40 and the highly amyloidogenic Aβ1-42. Although, there is considerable evidence indicating that Aβ aggregation triggers Alzheimer's disease in humans, the mechanism leading to disease development is not well understood.

There have been several drugs developed for the treatment of AD, including several anticholinergic agents, which are currently marketed for the treatment of AD. However, there remains a need in the art for the development of additional therapeutic strategies for the treatment of Alzheimer's disease.

### SUMMARY OF THE INVENTION

The present invention is based on the surprising discovery that reduced IGF signaling is protective against Aβ toxicity. For example, Example 1 shows that in a mouse model of Alzheimer's disease, mice with reduced IGF signaling were protected from neurological Alzheimer's-like disease.

In one embodiment, the invention is directed to a method of treating a patient suffering from a gain of function disease wherein the method comprises administering to said patient a therapeutically effective amount of an agent that reduces IGF-1 signaling, wherein the gain of function disease is a neurodegenerative disease.

In another embodiment, the invention is directed to a method of treating Alzheimer's disease wherein the method comprises administering to said patient a therapeutically effective amount of an agent that reduces IGF-1 signaling.

In yet another embodiment, the invention is a method of inducing Aβ hyper-aggregation in a patient in need thereof comprising administering to said patient a therapeutically effective amount of an agent that reduces IGF-1 signaling.

In an additional embodiment, the invention is a method of reducing Aβ proteotoxicity in a patient in need thereof, wherein the method comprises administering to said patient a therapeutically effective amount of an agent that reduces IGF-1 signaling.

In a further embodiment, the agent that reduces IGF-1 signaling is selected from the group consisting of an agent that inhibits the binding of a natural ligand to an IGF-1R, an agent that reduces the level of IGF-1 in the serum, an agent that reduces the level of IGF-1 in the nervous system, an agent that reduces the expression of IGF-1R or a ligand thereof, an agent that inhibits the phosphorylation of IGF-1R, and an agent that activates a FOXO transcription factor. In certain aspects, the agent that reduces IGF-1 signaling is an agent that activates a FOXO transcription factor.

In another embodiment, the agent that inhibits the binding of a natural ligand to IGF-1R is a receptor antagonist.

In an additional embodiment the agent that activates a FOXO transcription factor is selected from the group consisting of an agent that increases deacetylation of the FOXO transcription factor, an agent that decreases phosphorylation of the FOXO transcription factor, an agent that promotes nuclear translocation of the FOXO transcription factor and an agent that increases binding to a FOXO transcriptional co-regulator.

In a further embodiment, the invention is a method of identifying an agent that reduces Aβ toxicity comprising contacting an IGF-1 signaling indicator with a test agent wherein a decrease in IGF-1 signaling indicates that the test agent reduces Aβ toxicity.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIGS. 1A-E shows that reduction of IGF Signaling Protects Mice from Aβ-Associated Behavioral Impairments. (A) Long-lived mice carrying one Igf1r copy were crossed with transgenic Alzheimer's disease (AD) model mice harboring two AD-linked mutated genes, APP_{swe} (containing the human Aβ sequence) and PS1ΔE9 to obtain offspring of four genotypes: (1) wild-type, harboring two Igf1r copies and no AD-linked transgenes (WT), (2) long-lived mice with one Igf1r copy and no AD-linked transgenes (Igf1r^{+/-}), (3) AD model mice with two Igf1r copies and both AD-linked transgenes (AD), and (4) mice that harbor one Igf1r copy and both AD-linked transgenes (AD;Igf1r^{+/-}). (B) Latency time for reaching the cued platform significantly decreased through the acquisition sessions (p=0, F=35.49, df=3) in mice of all genotypes (p>0.05, F=1.84, df=3, n=8, 15, 16, 18 for AD, AD;Igf1r^{+/-}, WT, and Igf1r^{+/-}, respectively), suggesting no impairment of learning. (C) Significant differences were observed among AD mice and their counterparts of the other genotypes in the submerged platform test (p=5E-4, two-way analysis of variance [ANOVA], F=7.71, df=3) and across the acquisition days (p=0.032, F=2.97, df=3, n=8, 15, 16, 18 for AD, AD;Igf1r^{+/-}, WT, and Igf1r^{+/-}, respectively). AD mice searched for a longer period of time (p<0.05. Fisher LSD) for the submerged platform. No difference was observed among the three other genotypes. (D) AD;Igf1r^{+/-} animals crossed the previous platform location significantly (p=0.024, Kruskal-Wallis, χ²=9.38, df=3) more times than their AD counterparts. (E) Mice older than the age of plaque formation of all genotypes were tested in a Rota Rod task. Animals of the different genotypes significantly differed in their performance (p<0.01, one-way ANOVA, df=3, F=4.25; n=31, 32, 29, and 28 individuals for AD, AD;Igfr^{+/-}, Igf1r+/−, and wild-type, respectively). AD mice performed worst among the four genotypes whereas AD;Igfr^{+/-} mice where partially rescued because they performed significantly better than AD animals (p<0.05, Tuckey LSD). No statistical difference appeared between AD;Igfr^{+/-} animals and the two control genotypes. In all behavioral tests, 11- to 15-month-old mice were tested and age-match controlled. Error bars represent mean and standard error of the mean (±SEM).

FIGS. 2A-I shows that reduced IGF Signaling Reduces Aβ-Associated Neuroinflammation; (A-H) Immunohistochemistry using GFAP antibody indicated reduced astrocytosis in brain sections of 12- to 13-month-old AD;Igf1r^{+/-} mice (D and H) compared with age-matched AD mice (C and G). (I) Image analysis confirmed the significance of the GFAP signal difference (six mice per genotype and 3 sections per animal were analyzed, p<0.05; error bars represent mean±SEM).

FIGS. 3A-K shows that reduced IGF Signaling Protects from Aβ-Associated Neuronal and Synaptic Loss. (A-H) Immunohistochemistry using NeuN antibody indicated that neural densities in the brains of 12- to 13-month-old AD;Igf1r^{+/-} (D and H), WT (A and E), and Igf1^{+/-} (B and F) mice were comparable, while remarkable neuronal loss was observed in brains of age-matched AD animals (C and G). (I) Image analysis of the NeuN signals indicated that neural density in both cortices and hippocampuses of AD animals was significantly lower compared with their age-matched WT counterparts (cortex: p<0.001, one-way ANOVA, F=16.03; hippocampus: p<0.05, Kruskal-Wallis χ²=9.36, df=3). No significant difference was observed among brains of AD;Igf1r^{+/-} and Igf1^{+/-} mice (six mice per genotype and three sections per animal were analyzed). (J and K) Immunohistochemistry using synaptophysin antibody revealed that AD;Igf1r^{+/-} mice exhibit significantly higher synaptic densities than their age-matched AD counterparts in both frontal (J) and hippocampal (K) brain regions (AD n=7, AD;Igf1r^{+/-} n=5). Error bars represent mean±SEM.

FIGS. 4A-B shows that reduced IGF Signaling Facilitates Aβ Hyperaggregation. (A) Thioflavin-S amyloid labeling showed similar Aβ plaque burden in brains of AD (panels III and VII) and AD;Igf1r^{+/-} animals (panels IV and VIII). Image analysis indicated that the Thioflavin-S signals are similar in brains of AD and AD;Igf1r^{+/-} mice, but significantly different from WT and Igf1^{+/-} mice (panel IX). Six 12- to 13-month-old animals per genotype were analyzed. (B) Aβ plaque signal density was measured using Aβ-specific antibody (82E1). The signal per area ratio in brains of AD;Igf1r^{+/-} animals (panels IV and VIII) was significantly higher (panel IX, p<0.05) compared with brains of age-matched AD animals (panels III and VII), indicating higher plaque compaction in brains of AD;Igf1r^{+/-} mice (six mice per genotype and three sections per animal were analyzed; DG, dentate gyms; NC, neocortex). Error bars represent mean±SEM.

FIGS. 5A-C shows electron microscopy data and in vitro kinetic aggregation assays which reveal densely packed Aβ aggregates in the brains of AD;Igf1r^{+/-} Mice. (A) Electron micrographs of immunogold-labeled Aβ amyloids in the cortex of AD and AD;Igf1r^{+/-} mouse brains at different ages. Gold-labeled amyloid and fibrillar Aβ structures can be observed in the higher magnification electron micrographs (right panels). The amyloid load similarly increased with age in both genotypes, but highly ordered, condensed amyloids were present in AD;Igf1r^{+/-} cortices (arrows) but not in the cortices of their AD counterparts. White scale bars represent 1 μm, black bars 200 nm. (B) Unbiased automated image processing indicates that median intensities of regions of interest (ROIs) around the gold particles labeling Aβ plaques of AD;Igf1r^{+/-} mice (black) are significantly (p<0.04) higher than the plaque intensities of age-matched AD animals (red), confirming the higher compaction state of Aβ plaques of AD;Igf1r^{+/-} (135 images [34,087 ROIs] of AD and 101 images [26,066 ROIs] of AD;Igf1r^{+/-} were collected and analyzed). (C) Using an in vitro kinetic aggregation assay to assess fibril load, 12- to 13-month-old AD;Igf1r^{+/-} mouse brain homogenates (blue) accelerated Thioflavin-T (ThT) monitored in vitro kinetic aggregation significantly (p=0.035) faster than homogenates of age-matched AD brains (brown), indicating more Aβ seeding competent assemblies in AD;Igf1r^{+/-} mouse brains. Inset: Statistical analysis of results obtained in (C). Error bars represent mean±SEM.

FIGS. 6A-E shows that AD Brains Contain More Soluble Aβ Oligomers Than Brains of AD;Igf1r^{+/-} Animals. (A and B) ELISA assay detected significantly higher amounts of soluble Aβ₁₋₄₀ (A) (p<0.001) and Aβ₁₋₄₂ (B) (p<0.005) in brain homogenates of 12- to 13-month-old AD mice compared with brains of age-matched AD;Igf1r^{+/-} animals. (C and D) Western blot analysis reveals no detectable difference in the amount of SDS-sensitive Aβ monomers and small oligomeric assemblies between AD and AD; 1gf1r^{+/-} brain homogenates. Asterisk (*) indicates significant difference from WT or Igfr1^{+/-} mice. (E) Native SEC indicated that Aβ dimers were mainly associated with large structures in brains of 16- to 17-month-old AD;Igf1r^{+/-} mice (panel iii) while more soluble in brains of age-matched AD animals (panel ii, arrowhead) (panels represent 6 AD and 6 AD;Igf1r^{+/-} animals that were analyzed). Loading of total samples onto the gel and subsequent WB analysis using 6E10, confirmed equal protein loading onto the column (panel i). Error bars represent mean±SEM (A, B, and D).

FIG. 7 is a schematic showing that IGF-1 Signaling Can Play Several Roles in Mitigating the Toxicity of Aβ. The digestion of APP creates Aβ monomers that spontaneously aggregate to form toxic oligomers in vivo. At least two biological mechanisms can detoxify Aβ oligomers: (1) conversion of toxic oligomers into monomers (disaggregation), and (2) conversion of toxic oligomers into less toxic, larger structures (active aggregation). Within scenario 1 IGF-1, signaling normally functions to reduce protein disaggregase. Therefore, reduction of IGF-1 signaling would result in less oligomers and more monomeric forms of Aβ due to the activation of protein disaggregases. Our results are inconsistent with this scenario because we find less oligomers, but equal amounts of monomeric Aβ. Alternatively, in scenario 2, IGF-1 signaling could normally function to reduce protective protein aggregases that convert toxic species into larger, less toxic forms. Thus, reduced IGF-1 signaling elevates aggregase activity that in turn reduces the load of toxic oligomers and increases the compaction of less toxic fibrils. In support of scenario 2, we observed less soluble oligomers and highly compact amyloid plaques in AD;Igf1r^{+/-} animals. Alternatively, (3) IGF-1 signaling could promote proteotoxicity and neuroinflammation in response to toxic Aβ assemblies. These results are also consistent with this proposed mechanism as much less neuroinflammation was observed in the brains of protected AD;Igf1r^{+/-} animals. Yet this lower inflammation rate could be directly related to the reduction of Aβ oligomers in these animals by increased aggregases. In scenario 4, reduction of toxic secondary factors, such as reactive oxygen species (ROS), might synergize with the production of toxic Aβ assemblies to promote neuronal loss. Consistent with this mechanism, Igf1r^{+/-} mice are much more resistant to oxidative damage than wild-type mice. Taken together, IGF-1 signaling could impinge at multiple steps on the path to neuronal loss and neurodegeneration in response to Aβ production and none of the interventions are mutually exclusive. Our data are most consistent with a model in which reduced IGF-1 signaling reduces the load of toxic Aβ structures, presumably dimers, which results in higher compaction of plaques, reduced neuroinflammation, and reduced neuronal loss.

FIGS. 8A-H shows that the production and processing of APP is not affected by reduced IGF signaling. Quantitative RT-PCR (qPCR) revealed that reduction in IGF signaling does not affect the expression level of the human APP_{swe} transgene in the experimental mice. (A-C) qPCR of human Ab normalized to actin levels. (D and E) B-actin controls indicate the linearity of the reaction. (F) 12-13 month old Ad and AD;Igf1r+/− mouse brains contain similar quantities of APP processing enzymes and their products. Western blot analyses of AB, APP, C-terminal fragment (APP CTFs), a secretase (ADAM17) and B secretase (BACE1) quantities in brain homogenates of mice of all four genotypes. 12-13 month old AD and AD;IGF1r+/− mouse brains contain similar quantities of APP process enzymes and their products. (G) Quantification of the western blots shown in FIG. 22F indicated that the total amounts of Ab and of APP CTFs were higher in AD and AD;IGF1r+/− mice compared to their WT and IGF1r+/− age matched counterparts, however, no significant differences could be detected among AD and AD;IGF1r+/− brain samples. (H) No differences in the quantities of ADAM17 and of BACE1 were apparent among mice brains of all genotypes.

FIGS. 9A-D shows initial behavioral analysis of AD mice in the context of reduced IGF signaling. (A) Reduced IGF signaling results in lower body weight through 16-17 months of age in both Igf1r+/− and AD:Igf1r+/− mice compared to their WT and AD counterparts. (B) and C) shows the results of preliminary water maze experiment which indicates that the orientation impairment of AD and AD;IGF1r+/− mice is apparent at 9 months of age or later. Eight animals per genotype were trained to find a hidden platform submerged 1.5 cm under opaque water in a watermaze. Average times of latency in day 2 and 3 after training are presented for WT and AD animals (B) and for Igf1r+/− and AD;Igf1r+/− mice (C). Mice of all genotypes swam at the water maze at nearly identical speeds as measured by the Ethovision software (corresponding FIGS. 1B, C).

FIG. 10A-C shows that reduced IGF signaling protects against neuronal loss due to Ab expression. (A-C) Total neuron counts of AD;IGF1r+/− mice were significantly (P<0.05) higher than those of AD animals both in cortices and hippocampus CA1 regions in young (A, 4-5 month), midlife (B, 12-13 month) and old (C, 16-17 month) ages.

FIG. 11 shows immunohistochemistry using the Aβ antibody 6E10 indicated that Aβ plaques appear at similar temporal fashion in brains of AD and AD;IGF1r+/− mice. No plaques were detected in the brains of 4-5 month old mice, a few dispersed plaques observed in the brains of 8-9 month old animals and abundant plaques were seen throughout the brains of 12-13 month old animals (scale bars 200 um). Insets: plaques detected in AD;IGF1r+/− mice cortices were focal and more condensed compared to those seen in cortices of AD animals.

FIGS. 12A-B shows Thioflavin-S staining and proteinase K treatment of amyloid plaques. (A) Double labeling of Aβ plaques using Aβ antibody (82E1, red channel) and Thioflavin-S (green channel) confirms the specificity of Thioflavin-S plaque labeling in AD and AD:Igf1r+/− mice (nuclei are labeled with DAPI (Blue)). (B) Proteinase K treatment shows higher sensitivity of plaques of AD animals compared to their AD:IGF1r+/− counterparts. Plaques stained with the Aβ antibody 82E1.

FIGS. 13A-K shows post-embedding immuno-electron microscopy (EM) and EM quantification of animal brains used in this study. (A) Post-embedding immune-electron microscopy indicated that Ab plaques of 12-13 month old AD;IGF1r+/− (right panel) are of higher order and density compared to those of their AD counterparts (left panel). Corresponding FIG. 5A. (B) No background Ab antibody. (C) Electron microscope analysis of Ab plaque density. Median object size and object number threshold signature for the image displayed in panels D, E and F. Shaded areas correspond to different threshold regimes and their respected segmented object size and total number of segmented objects. (D-F) Segmented object (circled) for the th1, th2, and th3 threshold levels in C (corresponding FIG. 5B). (G) Immuno-gold label curve, bottom) measured along the pixel line pointed by the arrow. A threshold of 0.05% is applied to the inverse of the intensity profile (gray curve, top) and gold particle segmentation threshold is estimated by finding the first two non zeroes (to the right and left moving away from the center of the scan, dashed lines). (H) Squares represent pixels corresponding to the segmented gold particle. (I) Resulting mask used for estimating median intensity. Scale bar 100 nm in b-d and 10 nm e-g. Corresponding FIG. 5B. (J) The distance between each gold particle and its closest neighbor was measured in all images of AD and AD:Igf1r+/− mice brains. Distance distributions in both genotypes was nearly identical, indicating no difference in antibody accessibility among the genotypes. (K) Brain homogenates of 4-5 month old (prior to plaque formation) AD and AD;Igf1r+/− mice seeded in vitro kinetic Ab aggregation assays with similar efficiencies (corresponding to FIG. 5C).

FIGS. 14A-D shows that no significant differences were observed in the non-aggregated Aβ₁₋₄₀ content in brain homogenates of 4-5 month old mice as measured by ELISA assay (corresponding to FIG. 6A). B. No Aβ oligomers were detected in brain cytosolic fractions of 12-13 month old mice of all genotypes as measured by western blot analysis using Aβ antibody 6E10 (corresponding to FIGS. 6C-D). C-D. Standard protein mixture was separated on the superdex 75 size exclusion column to calibrate proteins of what size are expected to exit the column in each fraction.

FIGS. 15A and B shows the results of hidden platform experiment which indicated memory deficiency in AD mice compared to their WT counterparts but not among AD:Igf1r+/− and Igf1r+/− mice (Mice age 9-12 month, *Pᵥₐₗᵤₑ<0.01) (Group sizes: WT (10), Igf1r+/− (8), AD (10), AD:Igf1r+/− (10).

FIG. 15C shows the results of an independent hidden platform assay which showed that the memory impairments of AD mice are not detectable at 3 months of age but become apparent and significant thereafter (*Pᵥₐₗᵤₑ<0.05) (7 animals per group for all genotypes).

FIG. 15D shows that no significant difference in memory performance could be detected among AD:Igf1r+/− and Igf1r+/− mice.

FIG. 15E shows that the results of RotaRod assay which indicated that AD mice exhibit a significant locomotory coordination impairment compared to their WT counterparts. This impairment was apparent throughout the experiment (*Pᵥₐₗᵤₑ<0.01).

FIG. 15F shows that AD:Igf1r+/− mice exhibited a small locomotion coordination deficiency compared to Igf1r+/− which was apparent only at 16-17 month of age (*Pᵥₐₗᵤₑ<0.01).

FIG. 15G shows that AD mice had impaired agility compared to matched WT animals as measured by the string agility test. This impairment was apparent at 6 month of age and later (*Pᵥₐₗᵤₑ<0.01).

FIG. 15H shows that no significant agility performance difference could be detected among AD:Igf1r+/− and Igf1r+/− animals.

FIG. 16A shows that reduced astrocytosis was observed in the dentate gyms (hippocampal substructure) of AD:Igf1r+/− mice (B) (DG—dentate gyms).

FIG. 16B shows densitometry analysis of the NeuN signals which indicated that neural density in cortices of AD animals was significantly (Pᵥₐₗᵤₑ<0.01) lower compared to their WT counterparts. No significant difference observed among cortices of AD:Igf1r+/− and Igf1r+/− mice.

FIG. 17A-B shows that densitometry analysis confirmed a significantly higher Aβ signal intensity in cortices of 12-13 month old AD mice compared to cortices of their AD:Igf1r+/− counterparts (Pᵥₐₗᵤₑ<0.02).

FIGS. 17C and D shows that no significant intensity difference could be seen in the hippocampuses of AD and AD:Igf1r+/− animals.

FIG. 18 is a schematic showing that temporally distinct mechanisms protect from Aβ toxicity. At young age (I), disaggregation/degradation mechanism clear spontaneously formed Aβ oligomers from the brain, preventing the formation of Aβ plaques. This mechanism is possibly regulated by one of the Heat Shock factors (HSF). An age-dependent decline in the activity of the primary disaggregation/degradation mechanism invokes the activation of a secondary active-aggregation mechanism later in life (II). This mechanism, which is apparently inactive in young animals, mediates the assembly of small toxic Aβ oligomers into highly aggregated structures of lower toxicity. The IGF signaling pathway negatively regulates the protective active-aggregation mechanism, possibly via FOXO transcription factors.

FIG. 19 shows that 12-13 month old AD:Igf1r+/− mice had reduced astrocytosis in the dentate gyms compared to their age matched AD counterparts (DG—dentate gyms).

FIG. 20A shows that post embedding immuno-electron microscopy indicated that Aβ plaques of 12-13 month old AD:Igf1r+/− (right panel) are of higher order and density compared to these of their AD counterparts (left panel).

FIG. 20B shows that no background Aβ gold labeling was detected in cortices of WT and of Igf1r+/− mice of all ages using Aβ polyclonal antibody.

FIG. 21 shows that no significant difference observed in the non-aggregated Aβ content in brain homogenates of 4-5 month old mice as measured by sandwich ELISA assay.

### DETAILED DESCRIPTION OF THE INVENTION

A description of preferred embodiments of the invention follows.

The words “a” and “an” are meant to encompass one or more, unless otherwise specified.

“Treating” or “treatment” refers to the alleviation, amelioration, prevention or delay of the onset of the symptoms, complications, or biochemical indicia of the gain of function disease or condition to be treated, such as Alzheimer's disease, or arresting or inhibiting further development of the disease. Symptoms, complications and biochemical indicia of Alzheimer's disease include, for example, memory impairment, neuroinflammation, neuronal loss and/or the presence to toxic Aβ oligomers in the brain.

A “therapeutically effective amount” is an amount which, alone or in combination with one or more other active agents, can control, decrease, inhibit, ameliorate, prevent or otherwise affect one or more symptoms of a disease or condition to be treated.

A “patient” is a human subject in need of treatment.

As used herein, the term “inhibiting” or “decreasing” or “reducing” encompasses causing a net decrease by either direct or indirect means. The term “increasing” or “promoting” means to cause a net gain by either direct or indirect means.

The term “agent” is used herein to denote a chemical compound, a mixture of chemical compounds, a biological macromolecule (including, for example, a nucleic acid, an antibody, a protein or portion thereof, e.g., a peptide), or an extract made from biological materials such as bacteria, plants, fungi, or animal (particularly mammalian) cells or tissues.

The term “antibody” encompasses monoclonal antibodies, polyclonal antibodies, humanized antibodies, chimeric antibodies, single-chain Fv (scFv), Fab fragment, F(ab′) fragments, intrabodies, and synthetic antibodies.

The term “natural ligand” in reference to the IGF-1 receptor is intended to mean a ligand that binds the receptor and occurs naturally in the organism. In human, natural ligands of the IGF-1 receptor include, for example, IGF-1 and IGF-2.

The term “small molecule” as used herein, is meant to refer to a chemical compound which has a molecular weight of less than about 5 kD. Small molecules can be an organic or inorganic chemical compounds.

The insulin/IGF signaling pathway regulates stress resistance, ageing and longevity. In mammals, the IGF-1 signaling pathway is mediated by the activity of IGF-1 and its membrane bound receptor, IGF-1R. The Type 1 IGF-1 receptor (referred to herein as “IGF-1R”) shares about 70% amino acid homology to the insulin receptor and shares some of its signaling pathways (Jones et al. (1995), Endocr. Rev., 16: 3-34 (1995); Riedermann et al. (2006), Endocrine-Related Cancer 13: S33-S43). A major regulator of IGF-1 is growth hormone (GH) which is released from the anterior pituitary. The human IGF-1 is a 70 amino acid polypeptide (the sequence is described for example in U.S. Pat. No. 5,231,178 and Rinderknecht et al. (1978) JBC, 253(8): 2769-2776). IGF-1R is located in various tissues including muscle, ovary, pituitary and the brain (Daftray et al. (2005), Experimental Biology and Medicine 230(5): 292-306).

IGF-1R is a transmembrane receptor kinase. The IGF-1R is composed of two identical extracellular alpha-subunits that mediate ligand binding, two identical beta-subunits with a transmembrane domain and an intracellular tyrosine kinase domain. The ligand-receptor interaction results in phosphorylation of tyrosine residues in the tyrosine kinase domain. Ligands for IGF-1R include, for example, IGF-1 and IGF-2. Tyrosine residues that are phosphorylated in IGF-1R include tyrosines at positions 1131, 1135 and 1136 (LeRoith et al., Endocr Rev 1995 April; 16(2), 143-63). After phosphorylation, the receptor kinase then phosphorylates various intracellular proteins, including, for example, insulin receptor substrate-1 and Shc, which in turn activate the phosphatidyl inositol-3 kinase and the mitogen-activated protein kinase signaling pathways, respectively.

Reduction in serum IGF-1 levels has been associated with increased longevity and protection from cancer (Suh et al. (2008) PNAS 105(9): 3438-3442). That caloric restriction results in reduced circulating IGF-1 has been described extensively in the literature (Smith et al., (1995), J Clin Endocrinol Metab. 80(2):443-9; Giani et al., J Gerontol A Biol Sci Med. 63(8): 788-797; Hiyashi et al., Exp. Gerontol. 43(9): 827-32). In addition to calorie restriction, several agents have been described as effective in reducing the activity of the IGF-1 signaling pathway.

The invention is based on the discovery that a reduction in IGF-1 signaling provides protection from the toxicity associated with the peptide, Aβ. Reduced IGF-1 signaling is associated with the formation of high molecular weight, densely packed aggregates of Aβ. These densely packed aggregates are less toxic than small oligomeric Aβ assemblies (Haass et al. (2007) Nat Rev Mol Cell Biol 8(2):101-12; Shankar et al. (2008). Nat Med 14(8): 837-42).

In some aspects, the invention is directed to a method of treating a patient suffering from a gain of function disease or disorder comprising reducing the activity of the IGF-1 signaling pathway in the patient. In another embodiment, an agent that reduces IGF-1 signaling is administered to said patient in a therapeutically effective amount. The terms “gain of function disorder,” “gain of function disease,” “gain of toxic function disorder” and “gain of toxic function disease” are used interchangeably. A gain of function disorder is a disease characterized by increased aggregation-associated proteotoxicity. In these diseases, aggregation exceeds clearance inside and/or outside of the cell. Gain of function diseases include, but are not limited to neurodegenerative diseases including, for example, disease associated with aggregation of polyglutamine, Lewy body diseases, amyotrophic lateral sclerosis, transthyretin-associated aggregation diseases, and Alzheimer's disease. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: extracellular aggregates of Aβ peptide and intracellular aggregates of the microtubule associated protein tau. Transthyretin-associated aggregation diseases include, for example, senile systemic amyloidoses and familial amyloidotic neuropathy. Lewy body diseases are characterized by an aggregation of α-synuclein protein and include, for example, Parkinson's disease.

In certain aspects, the invention is a method of treating a patient suffering from Alzheimer's disease comprising reducing the activity of the IGF-1 signaling pathway in the patient. In another embodiment, an agent that reduces IGF-1 signaling is administered to said patient in a therapeutically effective amount.

In a further aspect of the invention, the invention is directed to a method of promoting the fibrillization or aggregation of Aβ in the brain into densely packed aggregates (also referred to herein as “promoting hyper-aggregation of Aβ”) in a patient in need thereof. A densely packed aggregate is an insoluble aggregate of Aβ that is associated with less neurotoxicity than soluble, oligomers of Aβ (such as Aβ dimers). Methods for distinguishing high molecular weight aggregates of Aβ from small, oligomeric Aβ are known in the literature and specific examples of such methods are described below in the Examples section. A patient in need of such treatment can be, for example, a patient at risk for developing Alzheimer's disease or a patient suffering from Alzheimer's disease. A patient at risk for developing Alzheimer's disease can be a patient carrying a genetic mutation resulting in increased production of Aβ or another risk factor for Alzheimer's disease.

Another embodiment of the invention is directed to a method of reducing the toxicity associated with deposition of Aβ in the brain comprising reducing IGF-1 signaling. Deposition of Aβ in the brain is associated with the presentation of symptoms associated with Alzheimer's disease including, but not limited to, memory impairment, neuronal loss and neuroinflammation.

The present invention relates to a methods of treating a patient suffering from Alzheimer's disease or other gain of function disease (such as a neurodegenerative disease associated with proteotoxicity), a methods of reducing proteotoxicity, a methods of reducing Aβ proteotoxicity and methods of inducing Aβ hyper-aggregation comprising administering to a patient an agent that reduces IGF-1 signaling in said patient. The agent can be selected from the group consisting of a small molecule, an antibody, a peptide and a nucleic acid. IGF-1 signaling is reduced when there is a decrease in or an inhibition of the activity of IGF-1, IGF-1R or their downstream signaling pathways. These downstream signaling pathways include the phosphotidylinositol 3-kinase and MAP kinase pathways that in turn affect the activity of several transcription factors and other downstream proteins. It is to be understood that the agent used according to the inventive methods can be any agent that reduces IGF-1 signaling by any mechanism. Agents that reduce IGF-1 signaling and strategies for reducing IGF-1 signaling have been reviewed in Riederman et al. (2006), IGF1R Signaling and Its Inhibition, Endocrine-Related Cancer 13: S33-S36, the contents of which are herein incorporated by reference.

In one embodiment, the agent that reduces IGF-1 signaling inhibits the binding of a natural ligand to IGF1-R. In another embodiment, the ligand is IGF-1. In an additional embodiment, the agent inhibits the binding of a natural ligand to IGF-1R in the brain. The binding of a natural ligand to the receptor can be inhibited by the administration of an IGF-1R receptor antagonist (IGF-1R antagonist). An IGF-1R antagonist inhibits the binding of IGF-1R to its ligand by binding to the receptor and thus, blocking the ability of the natural ligand to bind to the IGF-1R. IGF-1R antagonists include competitive antagonists, non-competitive antagonists, uncompetitive antagonists and partial antagonists. In one embodiment, the IGF1R antagonist is a small molecule. Small molecule IGF-1R antagonists have been described, for example, in U.S. Pat. No. 6,337,338 and Haylor et al. (2000), J Am Soc Nephrology 11(11):2027-2035), the contents of which are herein incorporated by reference. In a further embodiment, the IGF-1R antagonist is a peptide. As used herein, the term peptide includes proteins and antibodies as well as molecules comprised of two or more amino acids. Peptides that act as IGF-1R antagonists have been described, for example, in U.S. Pat. No. 7,173,005 and U.S. Patent Application Publication No. 20060233804, WO 00/23469 and EP 639981, the contents of each of which are herein incorporated by reference. In another embodiment, the agent is an anti-IGF-1R antibody. Examples of anti-IGF-1R antibodies and methods for the synthesis thereof are described in U.S. Patent Application Publication No.'s. 20070243194, 20080181891, 20080187536 and 20080176881, the contents of which are herein incorporated by reference. In addition, the preparation of immunizing antigen, and polyclonal and monoclonal antibody production can be performed using any suitable technique, including, for example, a phage display. A variety of methods have been described (see e.g., Kohler et al., Nature, 256:495-497 (1975) and Eur. J. Immunol. 6:511-519 (1976); Milstein et al., Nature 266:550-552 (1977); U.S. Pat. No. 4,172,124; Harlow, E. and D. Lane, 1988, Antibodies: A Laboratory Manual, (Cold Spring Harbor Laboratory: Cold Spring Harbor, N.Y.); and Current Protocols In Molecular Biology, Vol. 2 (Supplement 27, Summer '94), Ausubel, F. M. et al., Eds., (John Wiley & Sons: New York, N.Y.), Chapter 11, 1991); the teachings of each of which are incorporated herein by reference.

In another embodiment, the agent that inhibits the binding of a ligand to IGF-1R binds to IGF-1 and prevents IGF-1 from binding to IGF-1R. Such an agent can be, for example, an antibody that binds IGF-1. In another embodiment, the agent can be a soluble form of recombinant IGF-1R (sIGF1-R) (for example, IGF1-R lacking the intracellular and transmembrane domains) or an anti-IGF-1 antibody. As will be understood by one of skill in the art, the sIGF-1R can have the amino acid sequence of the extracellular domain and can optionally have various modifications including one of more amino acid substitutions and/or post-translational modifications provided that such sIGF-1R molecules have IGF-1 binding activity, which can be assessed using methods known in the art. Such sIGF-1R molecules can be made, for example, using recombinant techniques. An example of a soluble IGF1-R has been described, for example, in WO9718241, the contents of which are herein incorporated by reference.

The agent that reduces IGF-1 signaling can also act by inhibiting the expression of IGF-1R or a natural ligand thereof. In one embodiment, the agent that inhibits expression of IGF-1R or an IGF-1R ligand is a nucleic acid. Examples of specific nucleic acids that inhibit expression of IGF-1R have been described, for example, in U.S. Patent Application Publication No.'s. 20070185319 and 20050255493, the contents of which are herein incorporated by reference.

In one embodiment, the agent that inhibits expression of IGF-1R or a natural ligand thereof is an antisense nucleic acid. The antisense nucleic acid can be RNA, DNA, PNA or any other appropriate nucleic acid molecule. A duplex can form between the antisense sequence and its complementary sense sequence, resulting in inactivation of the gene. The target gene of the present invention is a gene encoding IGF-1R or a ligand thereof. The antisense nucleic acid can inhibit gene expression by forming a duplex with an RNA transcribed from the gene, by forming a triplex with duplex DNA, etc. An antisense nucleic acid can be produced, for example, for any gene whose coding sequence is known or can be determined by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their uses are described, for example, in U.S. Pat. Nos. 6,242,258, 6,500,615, 6,498,035, 6,395,544 and 5,563,050, the contents of each of which are herein incorporated by reference.

In another embodiment, the nucleic acid is an RNA interfering agent. An “RNA interfering agent” as used herein, is defined as any agent that interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference (RNAi). The target gene of the present invention is a gene encoding IGF-1R or a ligand thereof.

In one embodiment, the RNA interfering agent is a siRNA. The siRNA can be chemically synthesized, can be produced by in vitro transcription, or can be produced within a host cell. In one embodiment, siRNA is a double stranded RNA (dsRNA) molecule of about 15 to about 40 nucleotides in length or about 15 to about 28 nucleotides or about 19 to about 25 nucleotides in length or about 19, 20, 21, or 22 nucleotides in length, and may contain a 3′ and/or 5′ overhang on each strand having a length of about 0, 1, 2, 3, 4, 5, or 6 nucleotides. The length of the overhang is independent between the two strands, i.e., the length of the overhang on one strand is not dependent on the length of the overhang on the second strand.

RNAi also includes small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand may follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein).

In addition to RNA, RNA interfering agents can also be comprised of chemically modified nucleotides and non-nucleotides, and also include molecules wherein a ribose sugar molecule is substituted for another sugar molecule or a molecule which performs a similar function. In addition, a non-natural linkage between nucleotide residues may be used, such as a phosphorothioate linkage. The RNA strand can be derivatized with a reactive functional group of a reporter group, such as a fluorophore. Exemplary derivatives are modified at a terminus or termini of an RNA strand, typically the 3′ terminus of the sense strand. Other exemplary derivatives incorporate nucleotides having modified carbohydrate moieties, such as 2′O-alkylated residues or 2′-O-methyl ribosyl derivatives and 2′-O-fluoro ribosyl derivatives. The RNA bases can also be modified, for example, they can be alkylated or halogenated. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can additionally be incorporated.

In another embodiment, the nucleic acid is a ribozyme or a deoxyribozyme. Ribozymes and deoxyribozymes have been shown to catalyze the sequence-specific cleavage of nucleic acid molecules. The cleavage site is determined by complementary pairing of nucleotides in the RNA or DNA enzyme with nucleotides in the target nucleic acid. Thus, RNA and DNA enzymes can be designed to cleave to a nucleic acid molecule, thereby increasing its rate of degradation [Cotten et al, EMBO J. 8: 3861-3866, 1989; Usman et al., Nucl. Acids Mol. Biol. 10: 243, 1996; Usman, et al., Curr. Opin. Struct. Biol. 1: 527, 1996; Sun, et al., Pharmacol. Rev., 52: 325, 2000].

In yet another embodiment, the agent that inhibits the expression of IGF-1R or a ligand thereof is a peptide or a small molecule. Examples of such agents are described, for example, in U.S. Patent Publication No. 20070129399; Parrizas et al. (1997), Endocrinology 138: 1427-1433; Blum et al. (2000), Biochemistry: 15705-15712; Blum et al. (2003), J Biol Chem 278: 40442-40451, the contents of which are herein incorporated by reference.

The IGF-1 signaling pathway can also be reduced by activating a FOXO transcription factor. FOXO refers to the members of the forkhead box, class O family of transcription factors, such as FOXO1 (Genbank Acc. No. NM019739 and NM002015), FOXO3 (Genbank Acc. No. NM019740 and NP001446) and FOXO4 (Genbank Acc. No. Ab032770). Exemplary FOXO transcription factor include FOXO1, FOXO3a, FOXO4 and FOXO6 (Lam et al. (2006), Biochemical Society Transactions 34(5):722-726). In certain aspects, the FOXO transcription factor is FOXO3a. The FOXO gene family is highly conserved in mammals and is expressed in neurons. The FOXO proteins are negatively regulated by the PI3 kinase/Akt pathway. The FOXO proteins can be regulated by post-translational modifications, such as deacetylation and phosphorylation. For example, when phosphorylated by serine/threonine protein kinase Akt/Protein Kinase B (at either threonine 32, serine 253 and/or serine 315 of Foxo3), FOXO is retained in the cytoplasm and has impaired nuclear transcriptional activity. When dephosphorylated, FOXO is translocated to the nucleus and promotes transcriptional activity. Methods of modulating FOXO have been described, for example, in U.S. Patent Application Publication No. 20060069049, WO 20070(38982, and U.S. Pat. No. 7,288,385 the contents of each of which are expressly incorporated by reference herein. Therefore, in one embodiment of the invention, an agent that increases the activity of a FOXO transcription factor is administered. In another embodiment the agent that activates a FOXO transcription factor is selected from the group consisting of an agent that increases deacetylation of a FOXO transcription factor, an agent that decreases phosphorylation of a FOXO transcription factor, an agent that increases translocation into the nucleus of the FOXO transcription factor and an agent that modulates a FOXO transcriptional co-regulator. Exemplary FOXO transcriptional co-regulators are 14-3-3, PGC-1a, SMK-1, Sir2 proteins (including, for example SIRT1), p300, HCF-1, and orthologues of any of thereof (Daitoku et al. (2004). PNAS 101(27): 10042-10047; Li et al. (2008) PLoS Biol 6(9): e233. doi:10.1371/journal.pbio.0060233; Lam et al. (2006)).

The activity of the IGF-1 signaling pathway can additionally be reduced by an agent that inhibits the phosphorylation of a tyrosine residue of IGF-1R. Such agents have been described, for example, in WO 02/102805 A1 and are reviewed in Riederman et al. (2006), Endocrine-Related Cancer 13: S33-S43, the contents of each of which are incorporated by reference herein.

In a further embodiment, the agent that reduces IGF-1 signaling is an agent that reduces the level of IGF-1 in the serum. In another embodiment, the agent reduces the level of IGF-1 in the brain. As described above, IGF-1R are found in the central nervous system. The activity of IGF-1 in the nervous system is mediated by peripheral IGF-1 and IGF-1 that is synthesized by neuorons and glia (Daftary et al. (2005)). Therefore, in one example, the agent can reduce the IGF-1 signaling by decreasing the production or increasing the clearance of IGF-1 in the periphery and/or by decreasing the production or increasing the clearance of IGF-1 produced in the brain. In one embodiment, the agent that reduces IGF-1 level in the serum or in the nervous system is an anti-IGF-1 antibody.

The agent that reduces IGF-1 signaling activity can also be a peptide aptamer. Peptide aptamers are peptides or small polypeptides that act as dominant inhibitors of protein function. The peptide aptamers can target IGF-1R or a natural ligand thereof. In one embodiment, the ligand is IGF-1. Peptide aptamers bind specifically to target proteins, blocking their function (Kolonin and Finley, PNAS (1998) 95:14266-14271). Peptide aptamers that bind with high affinity and specificity to IGF-1R or a ligand thereof can be isolated by a variety of techniques known in the art. Peptide aptamers can be isolated from random peptide libraries by yeast two-hybrid screens (Xu et al., PNAS (1997) 94:12473-12478). They can also be isolated from phage libraries (Hoogenboom et al., Immunotechnology (1998) 4:1-20) or chemically generated peptides/libraries.

The agent that reduces IGF-1 signaling can be administered by any means appropriate for the disease or condition to be treated. Such routes of administration include, for example, parenteral, topical, intravenous, oral, subcutaneous, intraarterial, intracranial, intracerebroventricular, intraperitoneal, intranasal or intramuscular means. The agent that reduces IGF-1 signaling can optionally be administered in combination with other agents that are at least partly effective in treating a gain of function disease, Alzheimer's disease, condition associated with proteotoxicity or condition associated by Aβ toxicity.

The invention also relates to a methods of treating a patient suffering from a gain of function disease or Alzheimer's disease, a method of reducing proteotoxicity, a method of reducing Aβ proteotoxicity and a method of inducing Aβ hyper-aggregation comprising administering to a patient an agent that reduces IGF-1 signaling in said patient in combination with the administration of an additional proteostasis regulator. The term “proteostasis regulator” refers to small molecules, siRNA and biologicals (including, for example, proteins) that enhance cellular protein homeostasis. For example, proteostasis regulators can be agents that influence protein synthesis, folding, trafficking and degradation pathways. Proteostasis regulators function by manipulating signaling pathways, including, but not limited to, the heat shock response and the unfolded protein response, or both, resulting in transcription and translation of proteostasis network components. Proteostasis regulators can enhance the folding, trafficking and function of proteins (for example, mutated proteins). Proteostasis regulators can also regulate protein chaperones by upregulating transcription or translation of the protein chaperone, or inhibiting degradation of the protein chaperone. Proteostasis regulators can influence the biology of folding, often by the coordinated increase in chaperone and folding enzyme levels and macromolecules that bind to partially folded conformational ensembles, thus enabling their progression to intermediates with more native structure and ultimately increasing the concentration of folded mutant protein for export. In one aspect, the proteostasis regulator is distinct from a chaperone in that the proteostasis regulator can enhance the homeostasis of a mutated protein but does not bind the mutated protein. In another embodiment, the agent that reduces IGF-1 signaling is administered in combination with a mechanistically distinct proteostasis regulator. A mechanistically distinct proteostasis regulator is a proteostasis regulator that enhances cellular proteostasis by a mechanism other than by reducing IGF-1 signaling. In addition, proteostasis regulators can upregulate an aggregation pathway or a disaggregase activity. Exemplary proteostasis regulators are celastrol, MG-132 and L-type Ca²⁺ channel blockers (e.g., dilitiazem and verapamil).

It is to be understood that when the agent that reduces IGF-1 signaling is administered in combination with a second agent (for example, a proteostasis regulator, agents used in the treatment of Alzheimer's disease or other condition associated by Aβ toxicity), both agents can be administered at the same time and/or both agents can be administered at different times or sequentially.

The form of the agent that reduces IGF-1 signaling or pharmaceutical composition comprising the agent used according to the inventive methods of treatment depends on the intended mode of administration and therapeutic application. The agent can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

For parenteral administration, pharmaceutical compositions or pharmacologic agents can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

The compositions can be prepared as injectable formulations, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions and pharmacologic agents described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. Topical application can result in transdermal or intradermal delivery. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

In a further aspect, the invention is a method of identifying an agent that reduces Aβ toxicity comprising contacting a test agent with an IGF-1 signaling indicator wherein a decrease in IGF-1 signaling decrease indicates that the test agent reduces Aβ toxicity. In another aspect, the invention is a method of identifying an agent that promotes Aβ hyperaggregation comprising contacting a test agent with an IGF-1 signaling indicator wherein a decrease in IGF-1 signaling indicates that the test agent reduces Aβ toxicity. The method of identification can be conducted in a cell-free system, a cell-based assay or in an organism. An IGF-1 signaling indicator is a parameter indicative of an interaction between IGF-1R and a ligand thereof or a parameter indicative of the level of IGF-1R or a ligand thereof. IGF-1 signaling indicators include, but are not limited to, phosphorylation of IGF-1R, expression of IGF-1R or a ligand thereof, binding to IGF-1R, inhibiting the binding of IGF-1R to a natural ligand, the level of IGF-1R or a ligand thereof in a sample, and activation of a FOXO transcription factor (e.g., by determining acetylation or phosphorylation state).

Test agents can be obtained from a wide variety of sources including libraries of synthetic or natural compounds. For example, numerous means are available for random and directed synthesis of a wide variety of organic compounds and other agents. Libraries of natural compounds in the form of bacterial, fungal, plant and animal extracts are also available or can be readily produced. Additionally, natural or synthetically produced libraries and compounds are readily modified through conventional chemical, physical and biochemical means, and can be used to produce combinatorial libraries. Pharmacologic agents can also be subjected to directed or random chemical modifications, such as acylation, alkylation, esterification, amidification, etc. to produce structural analogs. Test agents encompass numerous chemical classes, typically synthetic, semi-synthetic, or naturally-occurring inorganic or organic molecules. Test agents include those found in large libraries of synthetic or natural compounds.

In an example of a cell-free assay, the effect of a test agent on the interaction between IGF-1R and a ligand thereof can be measured. The interaction between IGF1-R and a ligand thereof (e.g., IGF-1) is measured in the presence and absence of a test agent, wherein an inhibition of the interaction between IGF-1R and its ligand in the presence of the test agent indicates that the test agent has the ability to reduce Ab toxicity in an organism.

In another embodiment, the method of identifying an agent that reduces Aβ toxicity is a cell-based assay. The cell can be of vertebrate, non-vertebrate, eukaryotic, prokaryotic, mammalian or non-mammalian origin. In one embodiment, a cell that expresses the IGF-1R is contacted with a test agent and the ability of the test agent to inhibit an IGF-1 signaling indicator is measured. In one embodiment, the IGF-1 signaling indicator is the level of IGF-1R or a ligand thereof. The level of IGF-1R or a ligand therefor can be measured by immunoassay, for example.

In a further aspect, the invention is a method of identifying an agent that reduces Aβ toxicity in an organism comprising:
a) Administering a test agent to an organism;
b) Measuring a change in an IGF-1 signaling indicator; wherein a reduction in IGF-1 signaling indicates that the agent reduces Aβ toxicity in an animal;
wherein the IGF-1 signaling indicator is selected from the group consisting of reduced expression of IGF-1R or a ligand thereof, reduced phosphorylation of IGF-1R, an affinity for IGF-1R or a reduced affinity of IGF-1R for a natural ligand.

The organisms for use in the method of screening include vertebrates and invertebrates, mammals and other mammals. In one embodiment, the organism is selected from the group consisting of Drosophila, C. elegans, mouse and rat.

Methods of measuring a change in an indicator of reduced IGF-1 signaling have been described in the art. For example, phosphorylation of IGF-1R can be measured by detecting a phosphorylated tyrosine residue using an anti-phosphotyrosine residue as described in U.S. Patent Application Publication No. 20070129399, or by a kinase receptor activation assay (as described, for example, in U.S. Patent Publication No. 20060233804). The level of IGF-1R or a ligand thereof in a sample can be measured using immunoassay (ELISA, radioimmunoassay or chemiluminescence), for example.

The invention is illustrated by the following examples which are not meant to be limiting in any way.

### EXAMPLES

### Example 1

### Reduced IGF-1 Signaling Delays Age-Associated Proteotoxicity in Mice

### Summary

The insulin/insulin growth factor (IGF) signaling (IIS) pathway is a key regulator of aging of worms, flies, mice, and likely humans. Delayed aging by IIS reduction protects the nematode C. elegans from toxicity associated with the aggregation of the Alzheimer's disease-linked human peptide, Aβ. IGF signaling was reduced in Alzheimer's model mice and it was discovered that these animals are protected from Alzheimer's-like disease symptoms, including reduced behavioral impairment, neuroinflammation, and neuronal loss. This protection is correlated with the hyperaggregation of Aβ leading to tightly packed, ordered plaques, suggesting that one aspect of the protection conferred by reduced IGF signaling is the sequestration of soluble Aβ oligomers into dense aggregates of lower toxicity. These findings indicate that the IGF signaling-regulated mechanism that protects from Aβ toxicity is conserved from worms to mammals and point to the modulation of this signaling pathway as a promising strategy for the development of Alzheimer's disease therapy.

### Introduction

Most cases of Alzheimer's disease (AD) exhibit sporadic onset during the seventh decade of life or later, whereas the fewer mutation-linked, familial cases typically manifest during the fifth decade. These temporal features, common to numerous neurodegenerative diseases, define aging as the major risk factor for the development of these maladies (Amaducci and Tesco, 1994). The insulin/insulin-like growth factor (IGF) signaling (IIS) pathway regulates stress resistance, aging and is a life span determinant. IIS reduction results in stress-resistant, long-lived worms (Kenyon et al., 1993), flies (Tatar et al., 2001), and mice (Bluher et al., 2003, Holzenberger et al., 2003) and correlates with increased longevity of humans (Flachsbart et al., 2009, Suh et al., 2008, Willcox et al., 2008). Delayed aging, by IIS reduction, protects worms from proteotoxicity associated with the aggregation of the Huntington's disease-associated polyQ peptide (Morley et al., 2002) and the AD-linked human Aβ peptide (Cohen et al., 2006). However, little is known about whether this protection from proteotoxicity is conserved from worms to mammals, and what protective mechanisms may be operating.

Aβ originates from the endoproteolysis of the amyloid precursor protein (APP) (Glenner and Wong, 1984, Selkoe, 2004). The serine protease BACE (beta amyloid cleaving enzyme) cleaves APP (Farzan et al., 2000), followed by an intramembrane cleavage of the resulting fragment by presenilin 1 (PS1), an active component of the γ-secretase proteolytic complex (Wolfe et al., 1999). These events release the Aβ family of aggregation-prone peptides, including Aβ₁₋₄₀ and the highly amyloidogenic Aβ₁₋₄₂. Although compelling data indicate that Aβ aggregation triggers AD, the mechanism leading to the development of the disease is unclear (Selkoe, 2004). Recent studies suggest that it is not fibrils, but small Aβ oligomers lead to toxicity in AD model organisms (Cohen et al., 2006, Lesne et al., 2006) and to AD in humans (Haass and Selkoe, 2007, Shankar et al., 2008).

In the C. elegans Aβ model (Aβ worms [Link, 1995]), the protection from human Aβ₁₋₄₂ proteotoxicity conferred by IIS reduction is dependent upon two transcription factors, heat shock factor 1 (HSF-1), which regulates Aβ disaggregation, and DAF-16 (ortholog to FOXO in mammals), which facilitates the formation of larger, less toxic Aβ aggregates. Accordingly, Aβ worms protected from Aβ toxicity by reduced IIS accumulate more large Aβ aggregates and have fewer oligomers than did their unprotected counterparts with normal IIS (Cohen et al., 2006).

Although reduced IGF signaling extends the life span of mice (Holzenberger et al., 2003), IGF-1 infusion protects from Aβ toxicity (Carro et al., 2002, Carro et al., 2006), raising the query of whether IGF signaling reduction or activation protects from Aβ toxicity. To address this question, we created an AD mouse model with reduced IGF signaling by crossing a well-established AD transgenic mouse model (Jankowsky et al., 2001) with long-lived mice harboring only one Igf1r gene copy (Igf1r^{+/-} mice) (Holzenberger et al., 2003).

### Results

Creation of Mice with AD Transgenes in the Context of Reduced IGF-1R Signaling

Igf1r is the mammalian ortholog of the sole worm insulin/IGF receptor daf-2 (Kimura et al., 1997). Igf1r^{+/-} mice exhibit reduced IGF-1 signaling, are long-lived, oxidative stress resistant, and have reduced body size (Holzenberger et al., 2003). The AD mouse model expresses two AD-linked mutated transgenes, APP_{swe} (a humanized mouse APP that contains the human Aβ peptide sequence) and human presenilin-1 ΔE9, both driven by the mouse prion protein promoter (hereafter referred to as AD mice) (Jankowsky et al., 2001). The expression of these transgenes results in the production of human Aβ amyloid, plaque formation in the brain, and slow, progressive AD-like symptoms (Jankowsky et al., 2004). The AD-like mice also exhibit age-onset behavioral impairments, analogous to other AD murine models (Reiserer et al., 2007). The AD model is less aggressive than other AD models, exhibiting appearance of Aβ plaques in the brain at 6-7 months of age (Jankowsky et al., 2004). The slow onset of AD-like symptoms allows for the perturbation of IIS to examine its role in the age onset requirements of the AD-like syndrome.

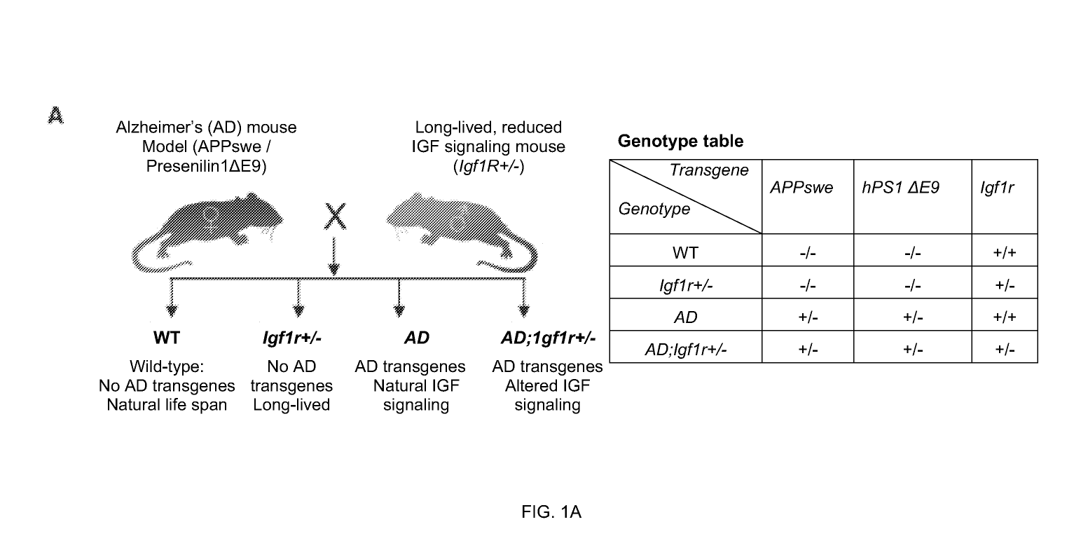

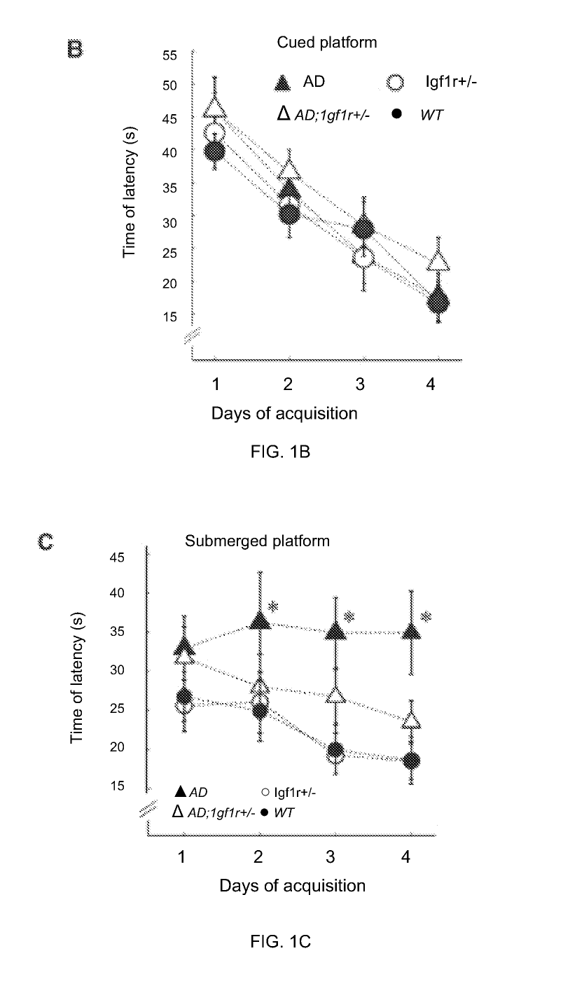

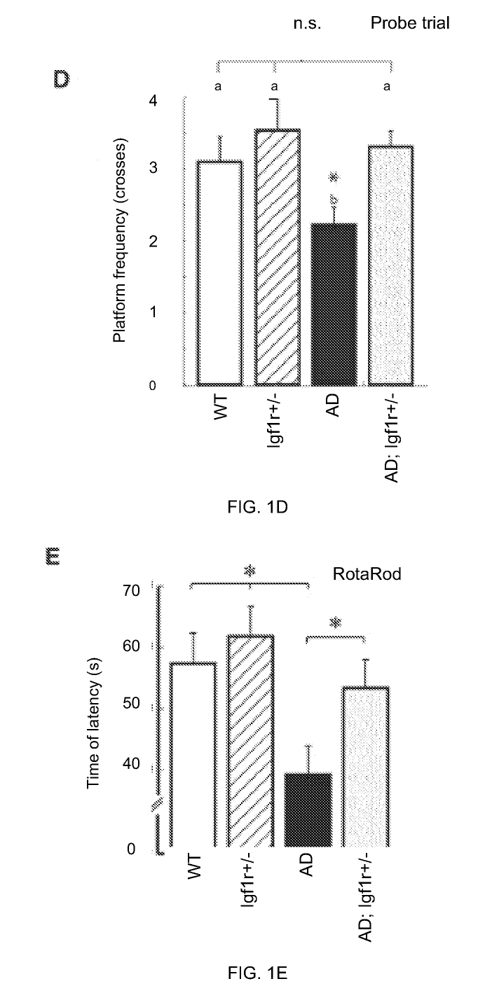

To equalize the genetic background of our mice, we first backcrossed both the AD and Igf1r^{+/-} mouse strains with wild-type 129 females for three generations, followed by four intercrosses between the AD and Igf1r^{+/-} mice. Crossing Igf1r^{+/-} with the AD mice generated offspring of four genotypes (FIG. 1A): The original parental genotypes, (1) heterozygous Igf1r^{+/-} (Igfr^{+/-}) and (2) AD mice, which served as internal controls (AD). (3) Congenic siblings that age naturally due to two Igf1r gene copies but carrying neither of the AD transgenes. These animals served as negative internal controls for asymptomatic AD-like disease and natural IGF-1 signaling (WT). Finally, (4) mice harboring both AD transgenes and only one Igf1r gene copy served as the experimental group of focus (AD;Igf1r^{+/-}).

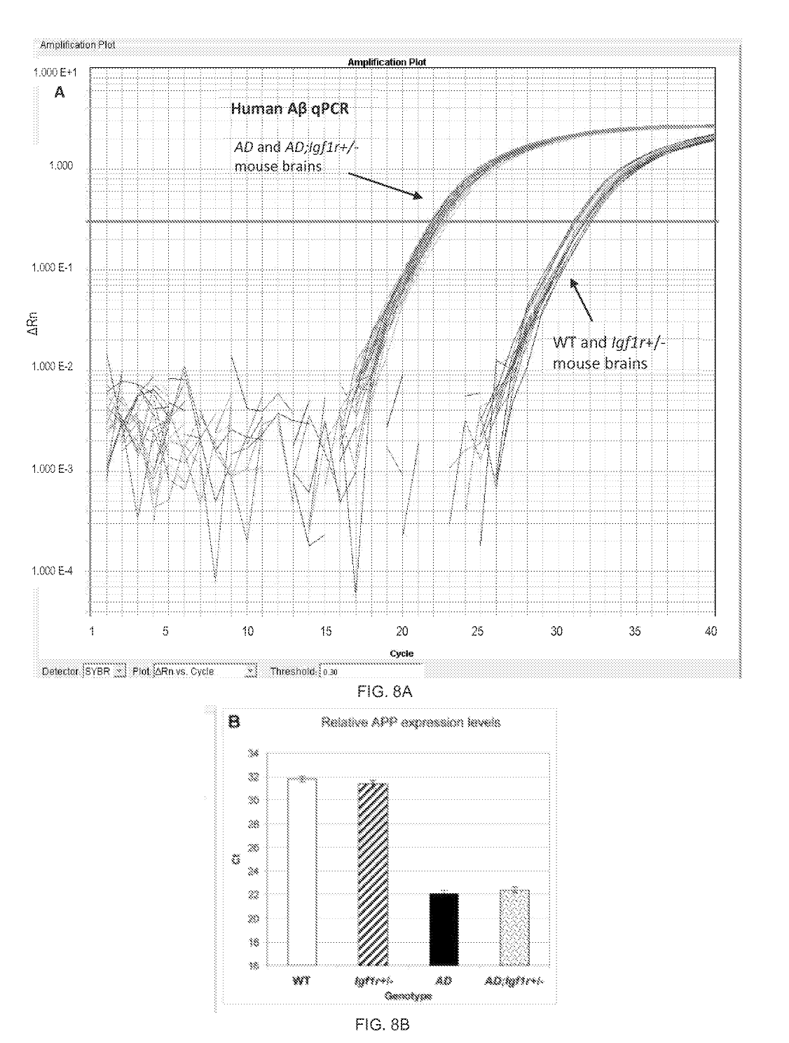

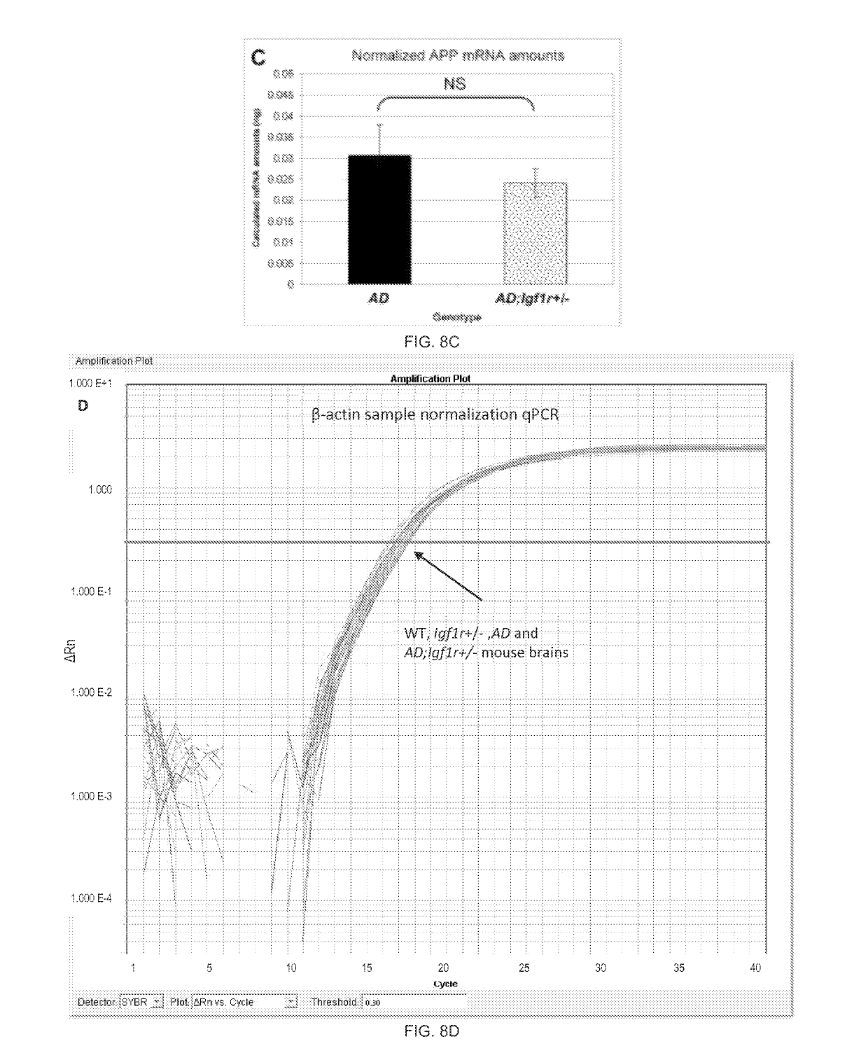

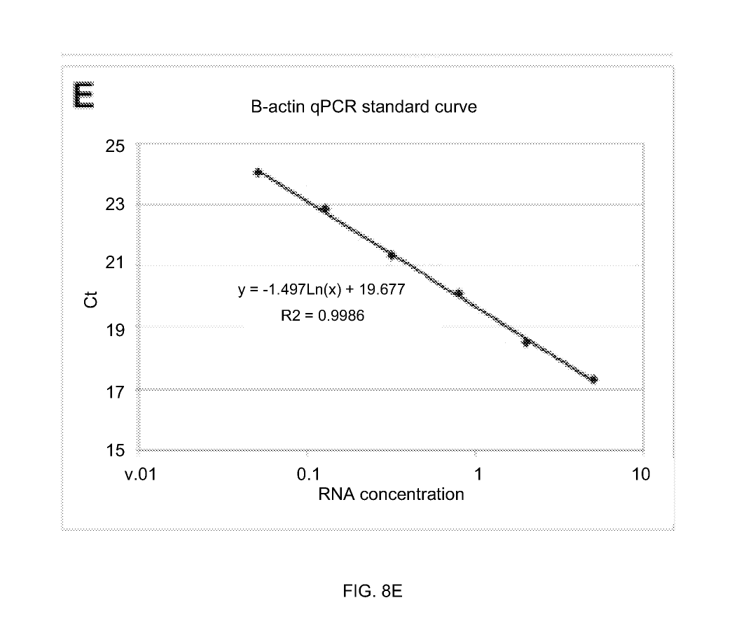

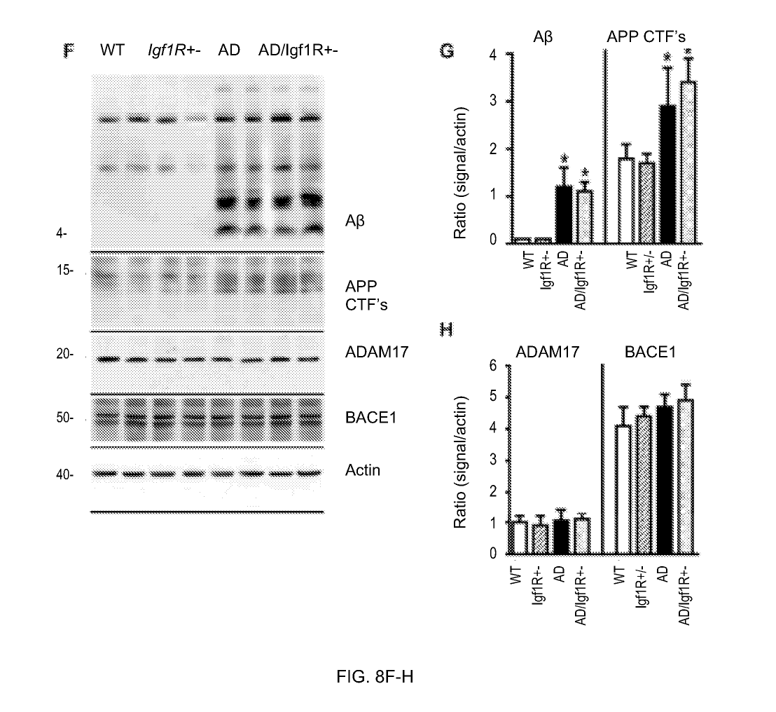

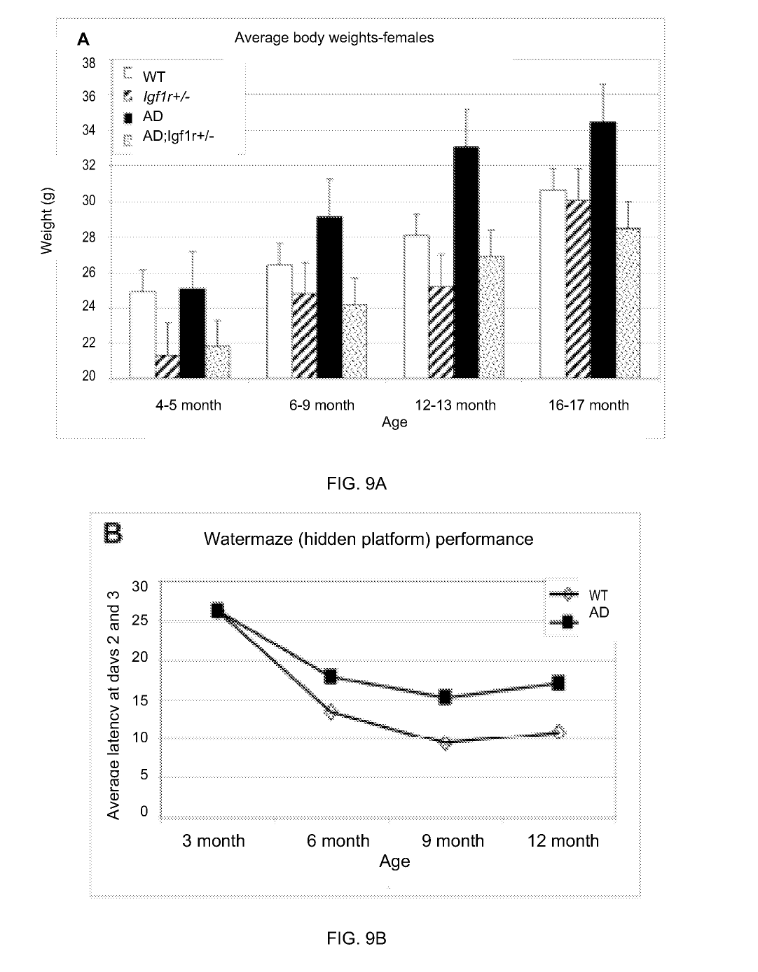

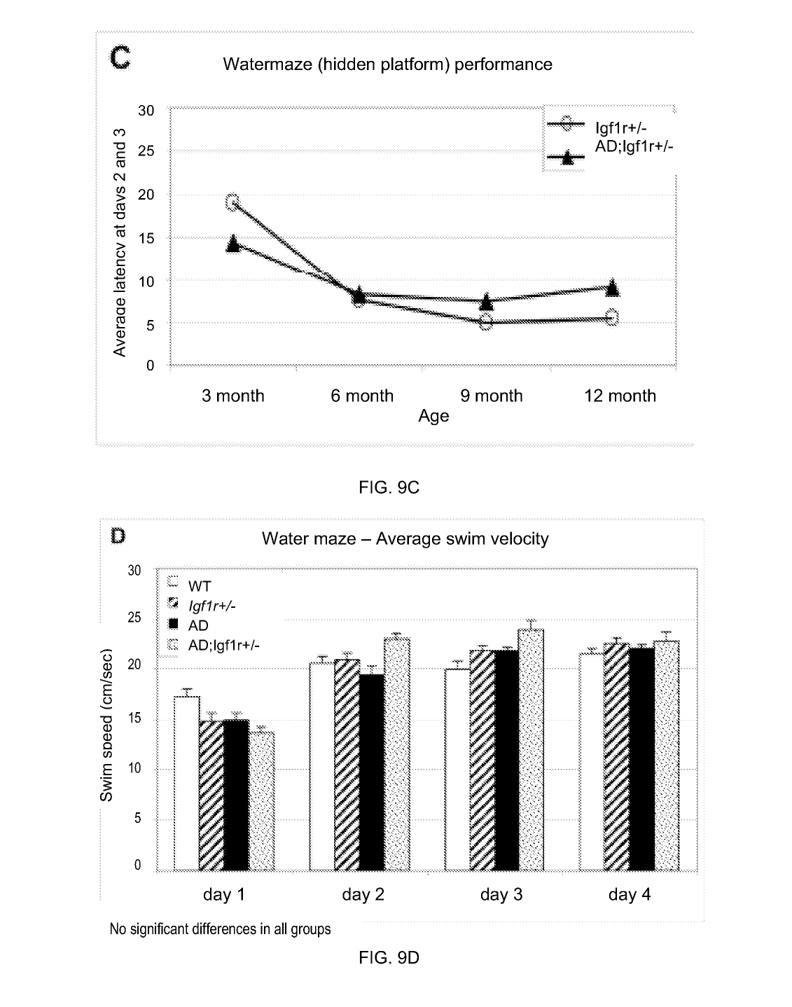

Quantitative polymerase chain reaction (PCR) analysis revealed that the expression levels of the APP_{swe} transgene were nearly identical in brains of AD and AD;Igf1r^{+/-} mice (FIG. 8A-E), indicating that IGF signaling reduction does not affect the expression of the prion protein promoter-driven transgenes. The levels of monomeric Aβ and of the C-terminal APP fragment (APP CTF) were also very similar in AD and AD;Igf1r^{+/-} mice (FIGS. 8F and 8G). Similarly, reduced IGF signaling did not affect the endogenous α and β secretases (ADAM17 and BACE, respectively) in mouse brains of all genotypes (FIG. 8 H). Together these results indicate that IGF signaling reduction affected neither the transgene expression nor the levels of the endogenous APP processing enzymes or their activity. As expected, both Igf1r^{+/-} and AD; Igf1r mice were smaller compared with their littermates carrying two Igf1r copies, indicating reduced IGF-1R signaling (FIG. 9A) (Holzenberger et al., 2003).

### Reduced IGF-1R Signaling Reduces the Behavioral Deficits of AD Mice

Age-onset memory deficiency and impairment of orientation and locomotion are associated with Aβ production in numerous AD murine models (Jensen et al., 2005, King and Arendash, 2002, Westerman et al., 2002). We evaluated whether reduced IGF-1 signaling protects mice from Aβ-associated behavioral impairments using several behavioral assays. As an initial analysis, we used eight animals per genotype and followed their performance in the Morris water maze test at 3, 6, 9, and 12 months of age, and found the greatest differences among AD, AD;Igf1r^{+/-} animals and their littermate controls at the 9 and 12 month time points (FIGS. 9B and C). At 16 months we observed mortality in the AD group that was not present in the AD;Igf1R^{+/-} mice (data not shown). Thus, we refined our behavioral analysis to the 11-15 month of age using a larger cohort of animals.

We measured the learning ability of mice using a Morris water maze with a cued (visible) platform for four consecutive days. As previously reported for other AD model mice (Blanchard et al., 2008, Westerman et al., 2002), the AD mice did not exhibit a learning deficiency compared to their age-matched WT, Igf1r^{+/-} and AD;Igf1r^{+/-} counterparts (FIG. 1B, p>0.05). In order to test orientation aptitude, we removed the cue from the platform and recorded the latency time required for mice to locate the submerged platform for four consecutive days. At days 2, 3, and 4 of the experiment, AD mice required a significantly (p<0.05) longer time to find the hidden platform compared to their WT, Igf1r^{+/-} and AD;Igf1r^{+/-} counterparts (FIG. 1C). This indicates that, like other mouse AD models (Jensen et al., 2005, King and Arendash, 2002, Westerman et al., 2002), orientation capabilities of AD animals are impaired (swim velocities were nearly identical for all genotypes; FIG. 9D). Lastly, we tested memory skills by removing the platform from the water maze and recording the number of the crosses of the previous platform location (probe trial). AD mice crossed the platform's previous location significantly (p<0.05) fewer times than their WT, Igf1r^{+/-} and most importantly AD;Igf1r^{+/-} counterparts, indicating impaired memory. The observation that AD;Igf1r^{+/-} animals crossed the previous platform location at similar frequencies compared to WT and Igf1r^{+/-} animals suggests partial memory restoration (FIG. 1D).

Next, we tested the effect of reduced IGF-1 signaling on the motor skills of AD model mice using a Rota-Rod assay. Much like the orientation and memory tests, AD mice performed significantly less well than their age-matched WT, Igf1r^{+/-} and AD;Igf1r^{+/-} counterparts in this assay (FIG. 1E, p<0.05).

Collectively, the behavioral data revealed that AD mice have impaired orientation and memory performance as well as locomotion impairment that can be delayed by reduced IGF-1 signaling.

### Reduced IGF-1R Signaling Reduces Inflammation and Neuronal Loss in AD Mice

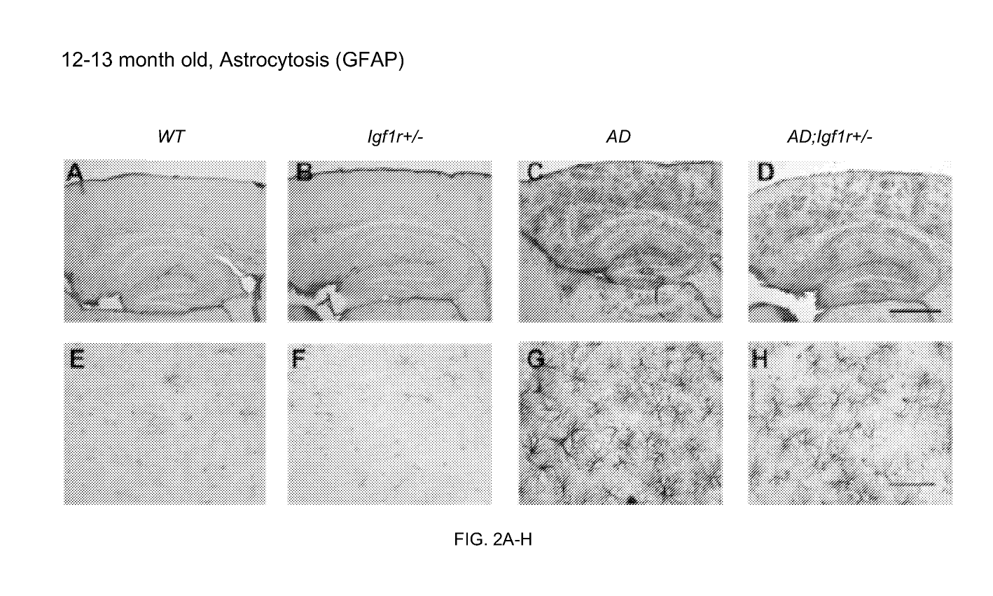

We asked whether the appearance of biological markers associated with AD-like disease in mice was also delayed by reduced IGF signaling. First we tested whether reactive astrocytosis, indicative of neuroinflammation, associated with AD in humans (Mancardi et al., 1983), and with Aβ aggregation in brains of AD model mice (Wirths et al., 2008), was reduced in AD;Igf1r^{+/-} animals. Utilizing glial fibrillary acidic protein (GFAP) antibodies, which recognize activated astrocytes (Mancardi et al., 1983), we found notably less activated astrocytes in the brains of AD;Igf1r^{+/-} mice compared with age-matched AD mice (FIG. 2). This reduction was apparent both in the cortex and hippocampus (FIG. 2I), indicating that neuroinflammation is reduced in AD;Igf1r^{+/-} mice compared with age-matched AD mice. Interestingly, whereas the GFAP signal observed in cortices of AD mice was largely diffuse, cortical GFAP staining of AD;Igf1r^{+/-} mice appeared to be focal (compare FIGS. 2C and 2D), suggesting that neuroinflammation within AD;Igf1r^{+/-} brains is confined to smaller areas than in the brains of AD animals.

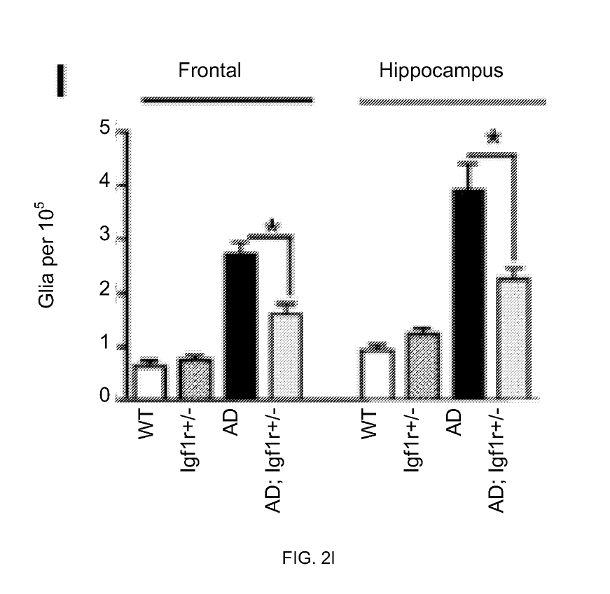

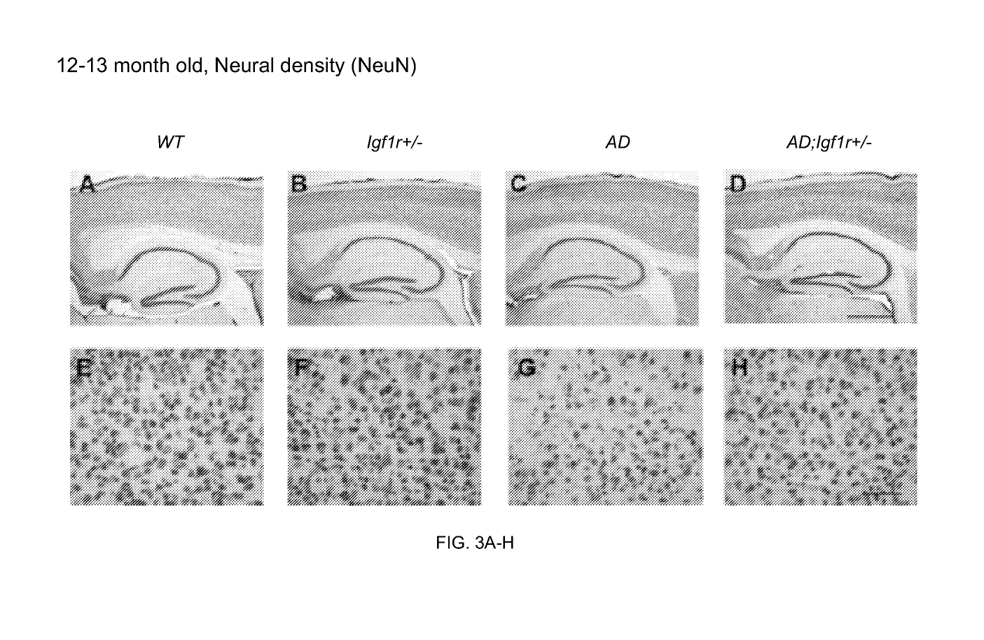

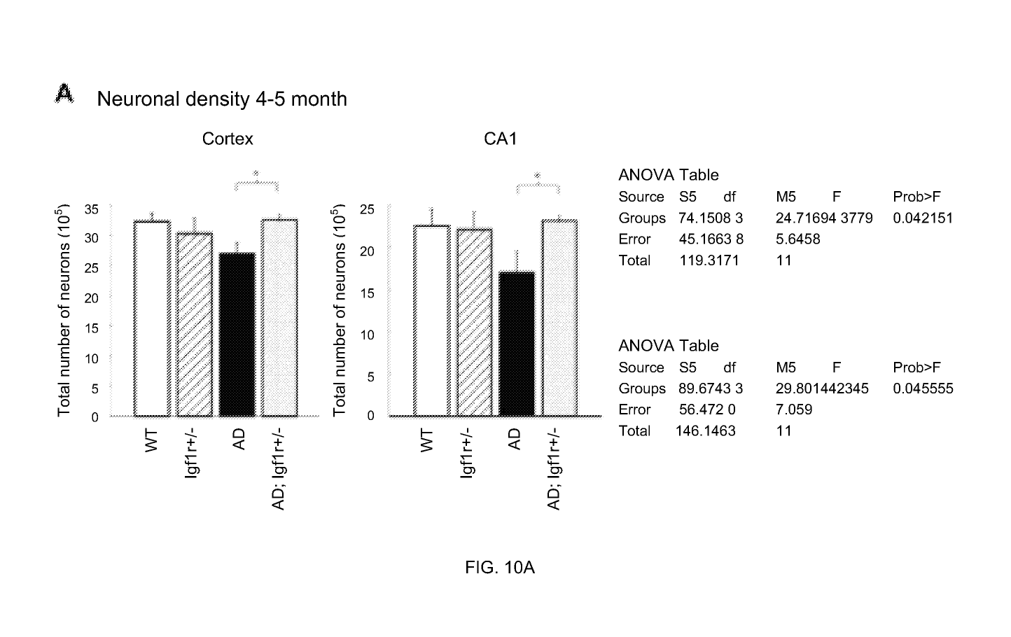

Neuronal loss is another hallmark of AD in humans (Scheff et al., 1990) and AD model mice (Masliah and Rockenstein, 2000). We used direct stereological visualization and NeuN immunoreactivity, a marker of neuronal density that declines in AD mice, and found higher NeuN immunoreactivity in the cortices of 12- to 13-month-old AD;Igf1r^{+/-} mice compared to their age-matched AD counterparts (FIG. 3). This indicates that reduced IGF signaling protects from neuronal loss. Similar neuronal losses were observed in young (4-5 months of age) and in old (16-17 months) AD but not in AD;Igf1r^{+/-} mice when compared to age-matched control genotypes (FIG. 10).

Reduced synaptic density is an additional hallmark and probably causative of AD (Hamos et al., 1989). Thus, we used the synaptic marker synaptophysin to compare synaptic densities in frontal and hippocampal brain regions of 12- to 13-month-old mice of all genotypes (Hamos et al., 1989) and found significantly lower synaptic densities in both brain regions (FIGS. 3J and 3K, respectively) of AD animals compared with their AD;Igf1r^{+/-} counterparts. These observations confirm that IGF signaling reduction protects mice form Aβ-associated neuronal loss.

### Reduced IGF Signaling Promotes the Formation of Densely Packed Aggregates

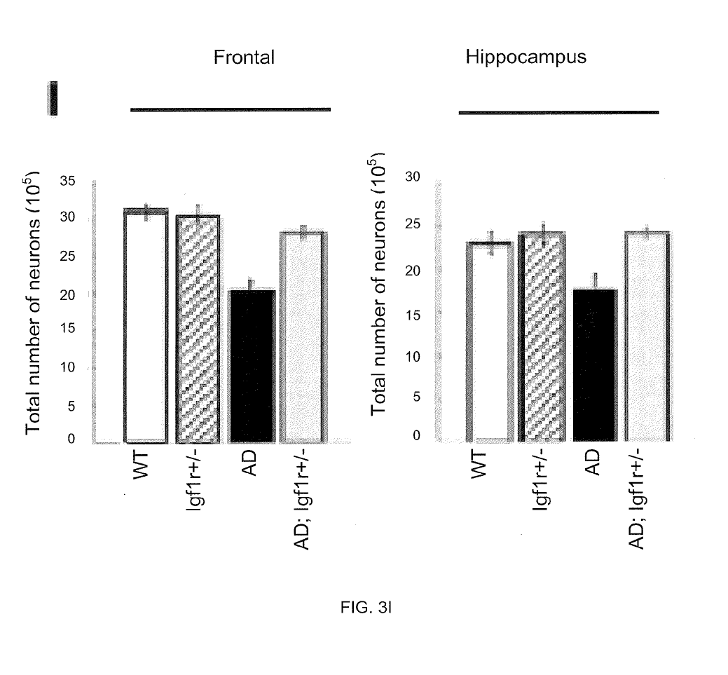

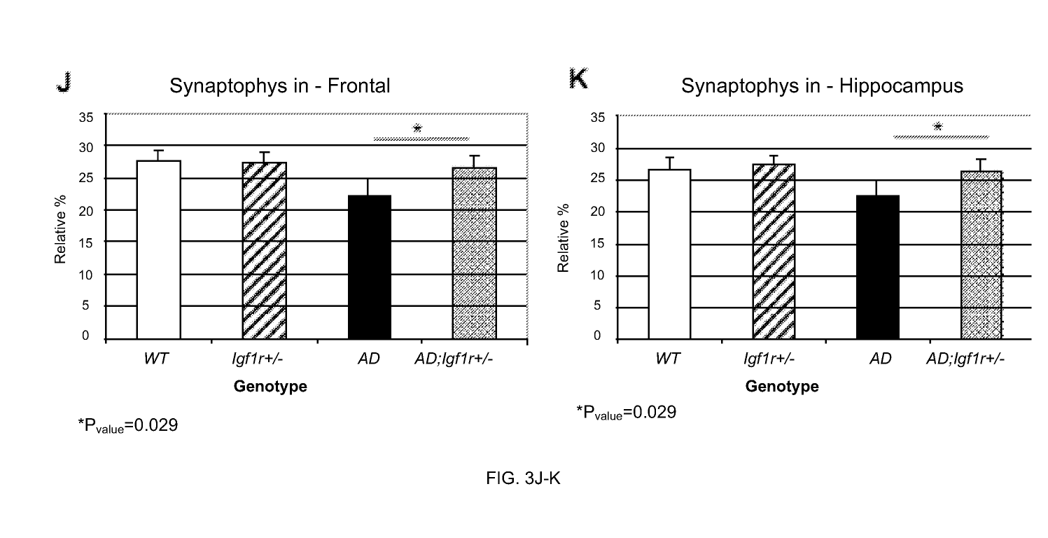

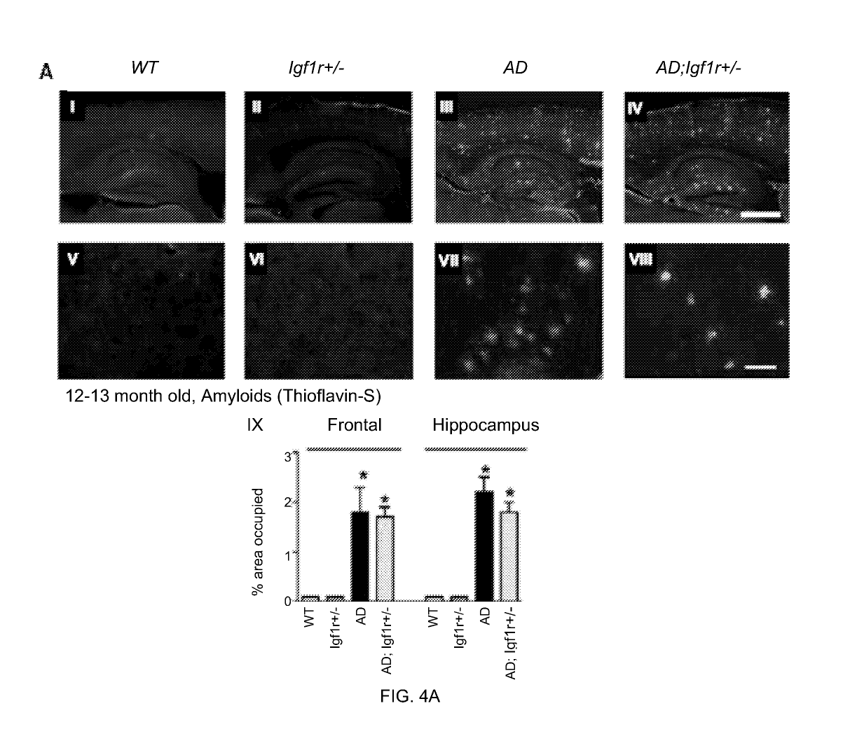

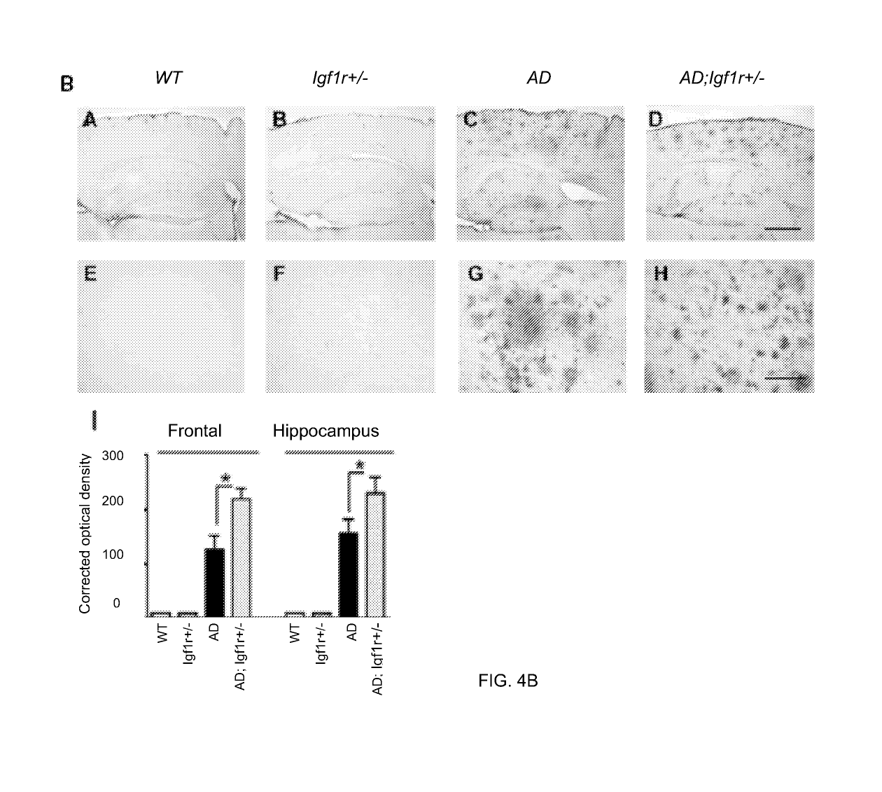

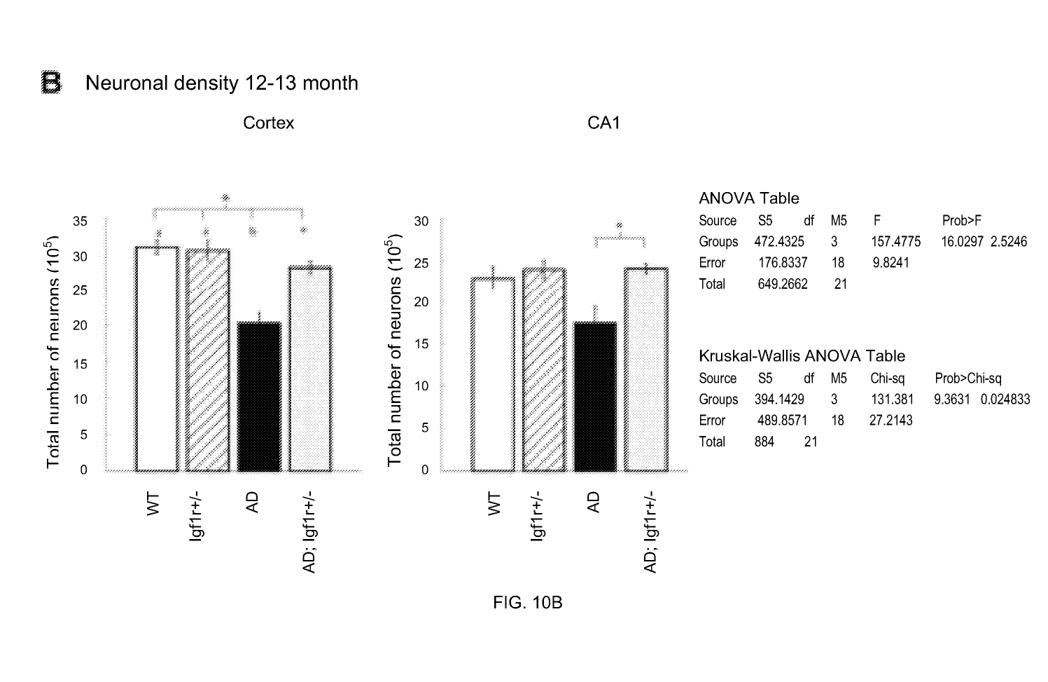

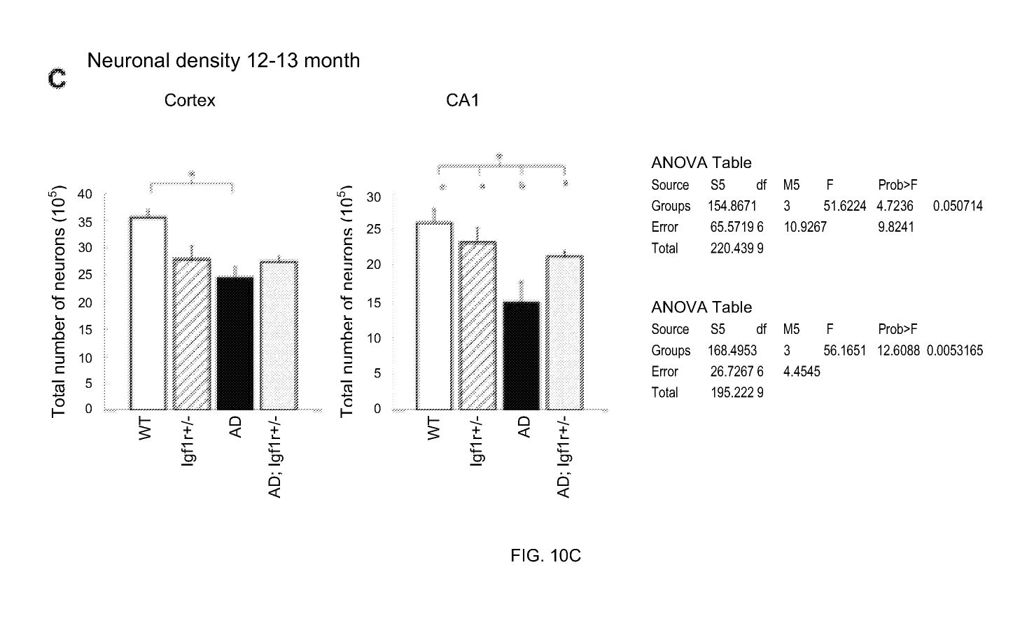

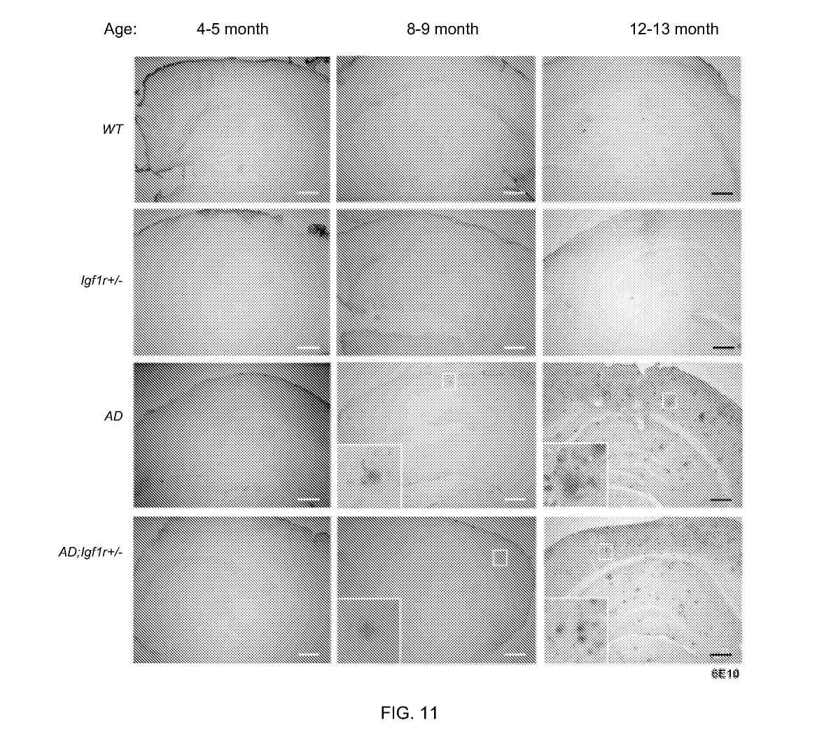

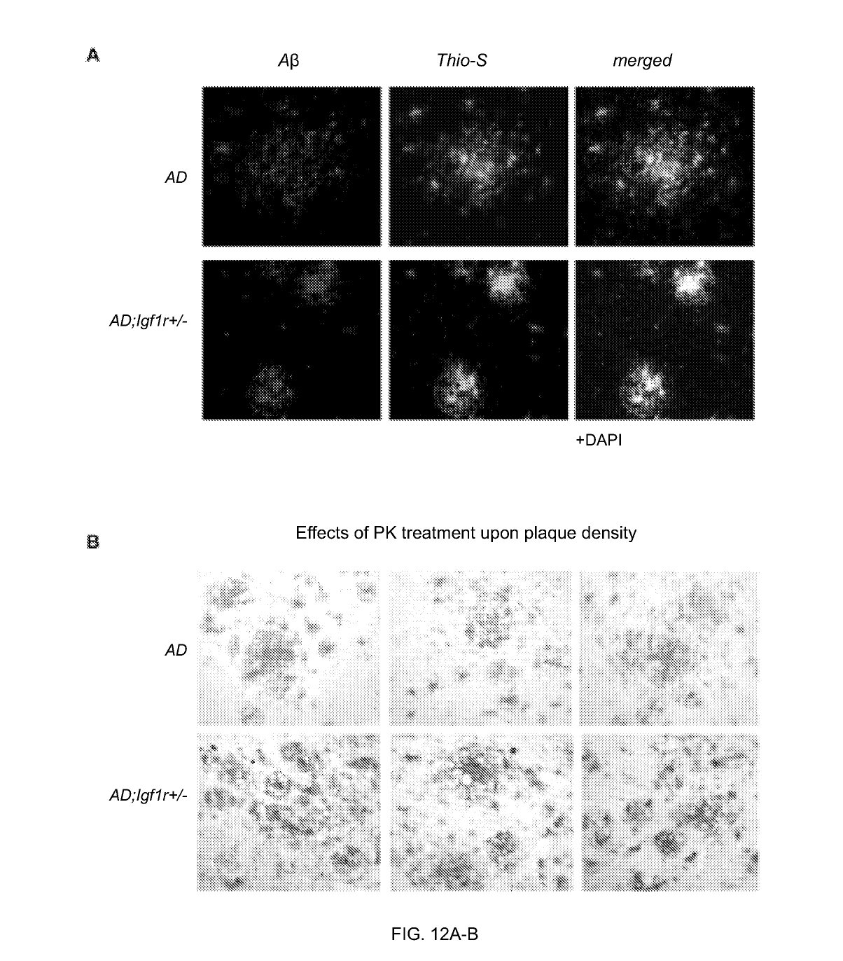

To explore the mechanism underlying the protection toward behavioral deficiencies conferred by reduced IGF signaling, as well as the protection from inflammation and neuronal loss in mice ectopically expressing mutated AD-linked transgenes, we investigated the nature of Aβ assemblies in brains of AD and AD;Igf1r^{+/-} mice. Immunohistochemistry (IHC) and Aβ antibodies (clone 6E10) were used to visualize Aβ plaques in brain sections of AD and AD;Igf1r^{+/-} mice (FIG. 11). Consistent with previous results (Jankowsky et al., 2004), Aβ plaques could not be detected in brains of young mice (4-5 months old). A few plaques were observed in the brains of 8- to 9-month-old animals, whereas the number of plaques increased in the brains of 12- to 13-month-old AD and AD;Igf1r^{+/-} mice. No background staining was observed in brains of WT or Igf1r^{+/-} mice at any age examined (FIG. 11). Thus, reduced IGF signaling has no apparent effect on the onset of plaque formation. Next we used the fluorescent dye Thioflavin-S to visualize amyloid within AD and of AD;Igf1r^{+/-} brains and found nearly identical amyloid load in cortex and hippocampus regions of both genotypes (FIG. 4A, panel IX). (Colocalization of Thiosflavin-S labeling with the signal of specific Aβ antibody [clone 82E1] confirmed the plaque specificity of Thioflavin-S FIG. 12). Therefore, the kinetics of Aβ plaque appearance as well as the amyloid load did not appear to differ between AD and AD;Igf1r^{+/-} animals.

Closer inspection of the Aβ plaques analyzed by IHC revealed that plaques observed in the cortices of AD;Igf1r^{+/-} animals are smaller and more condensed than those detected in the cortices of their age-matched AD counterparts (FIG. 11, 12-13 months, insets). To compare the plaque compaction in the mouse brains, we used the highly specific Aβ antibody (clone 82E1 that recognizes processed Aβ) and measured the Aβ immunoreactive optical density (signal per area) in the different brains. Significantly higher Aβ immunoreactive optical densities were detected in brains of AD;Igf1r^{+/-} mice than in AD brains (FIG. 4B, panel IX), suggesting higher compaction of the Aβ amyloid plaques in AD;Igf1r^{+/-} animals.

We also compared the protease sensitivity of plaques of AD and AD;Igf1r^{+/-} animals by treating brain sections of 12- to 13-month-old mice with 10 μg/ml proteinase K prior to their labeling with Aβ antibody. A diffuse staining of Aβ plaques seen in AD brain slices compared to the AD;Igf1r^{+/-} brain slices (FIG. 12 B) suggested that plaques of AD;Igf1r^{+/-} animals are more protease resistant than those of AD mice.

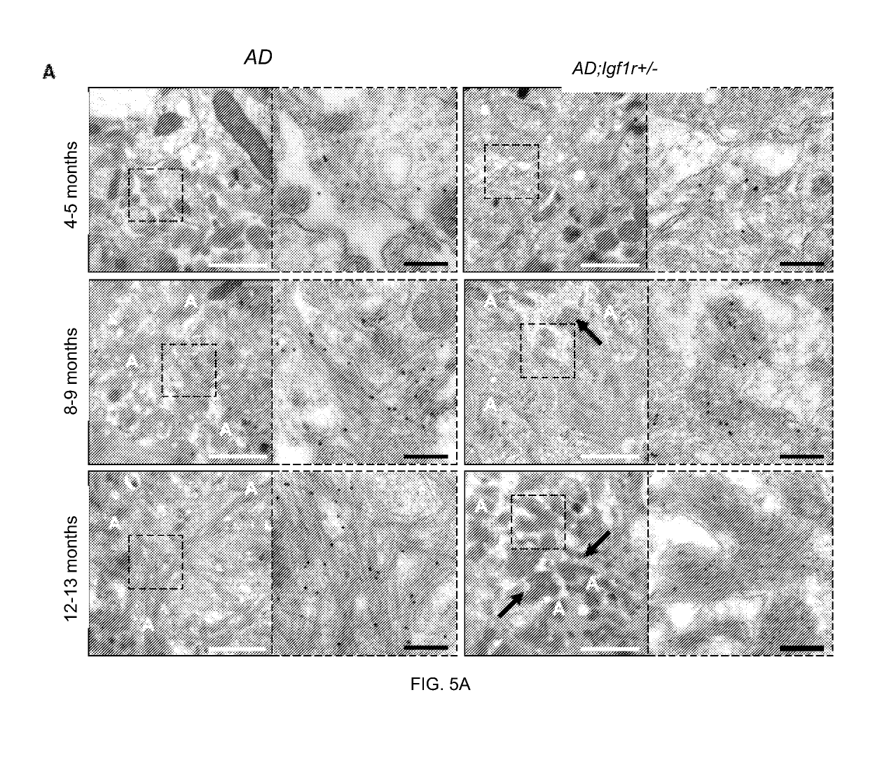

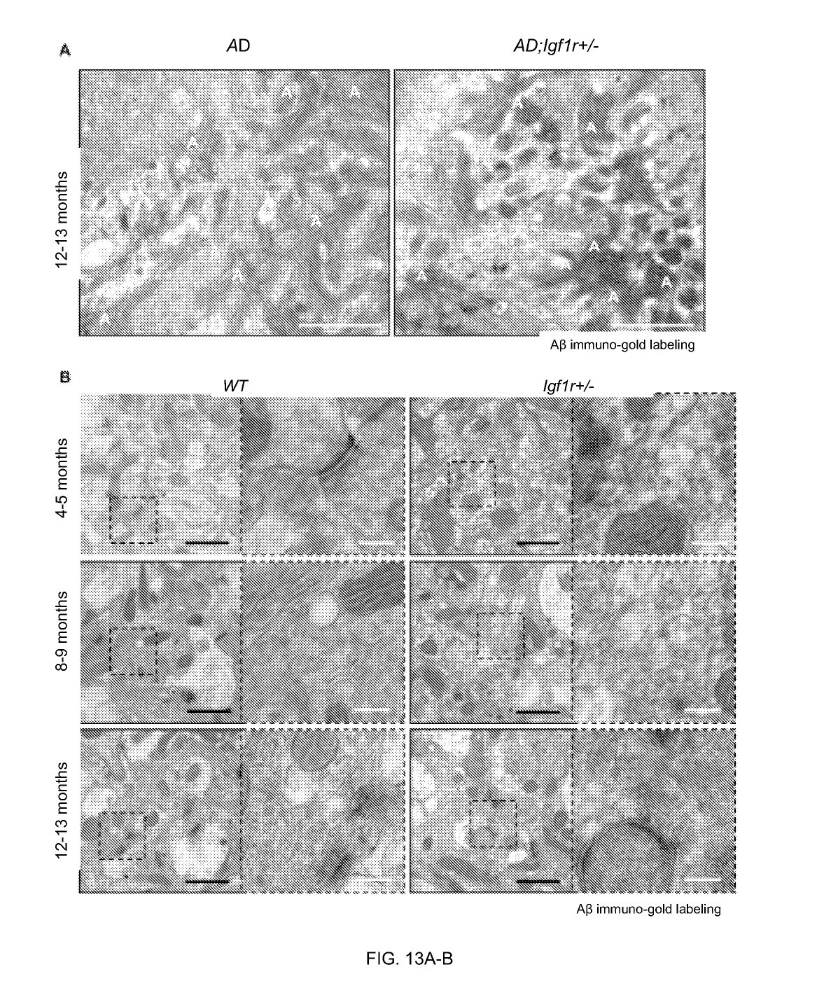

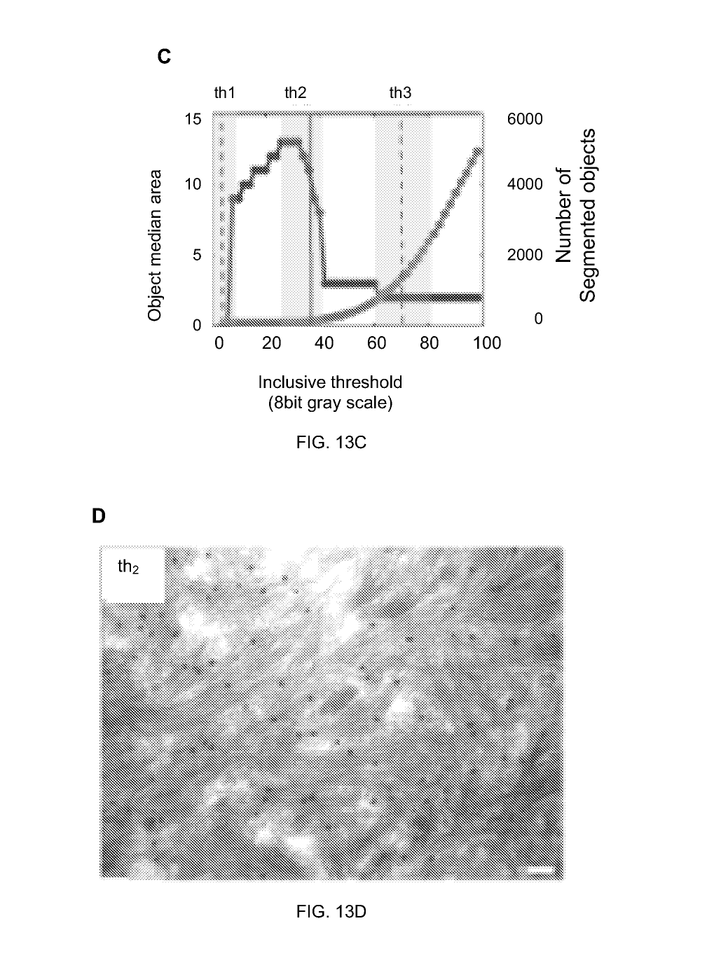

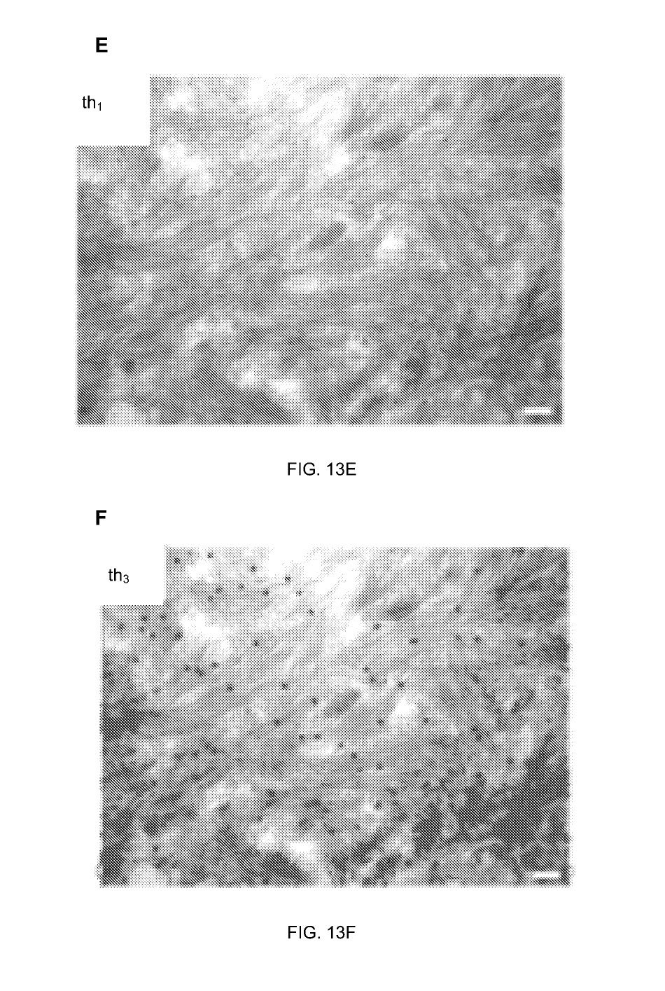

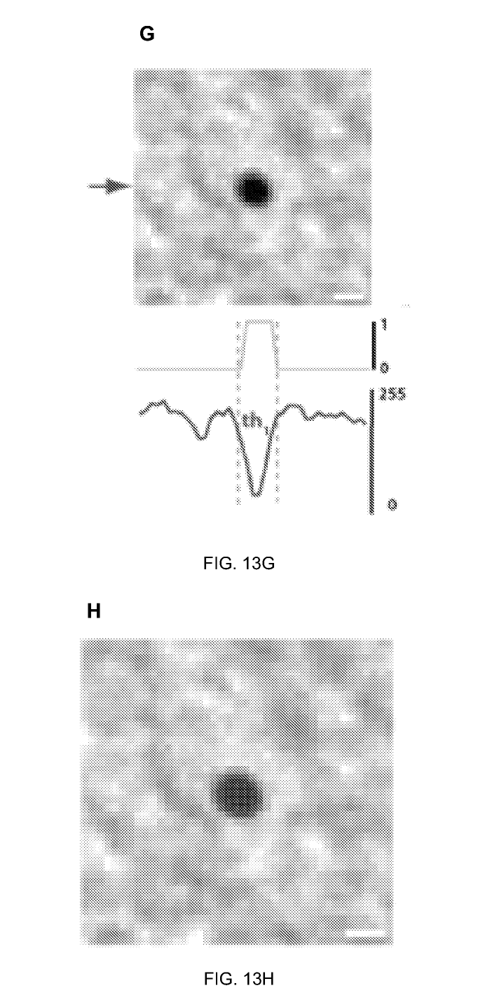

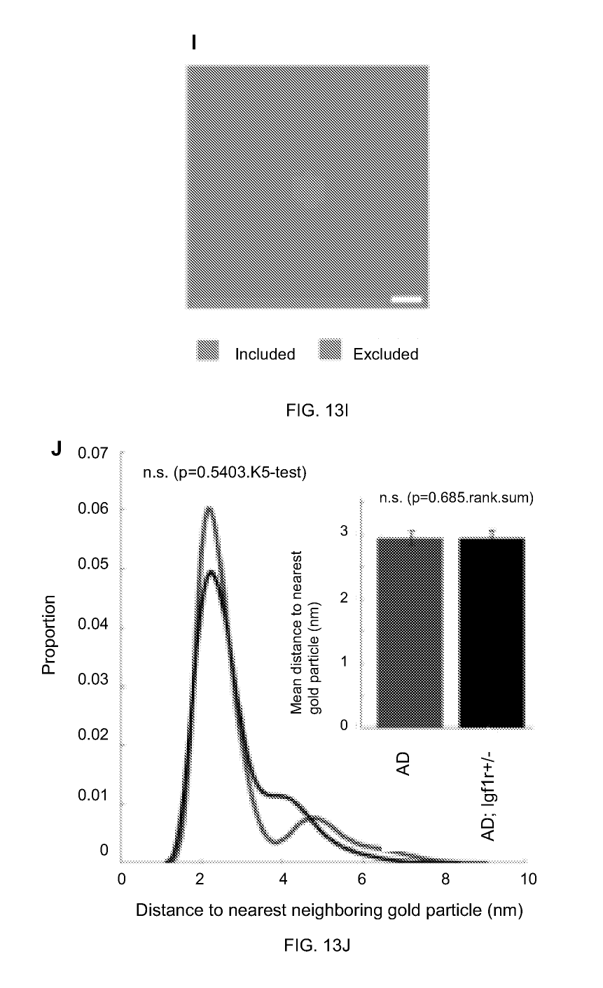

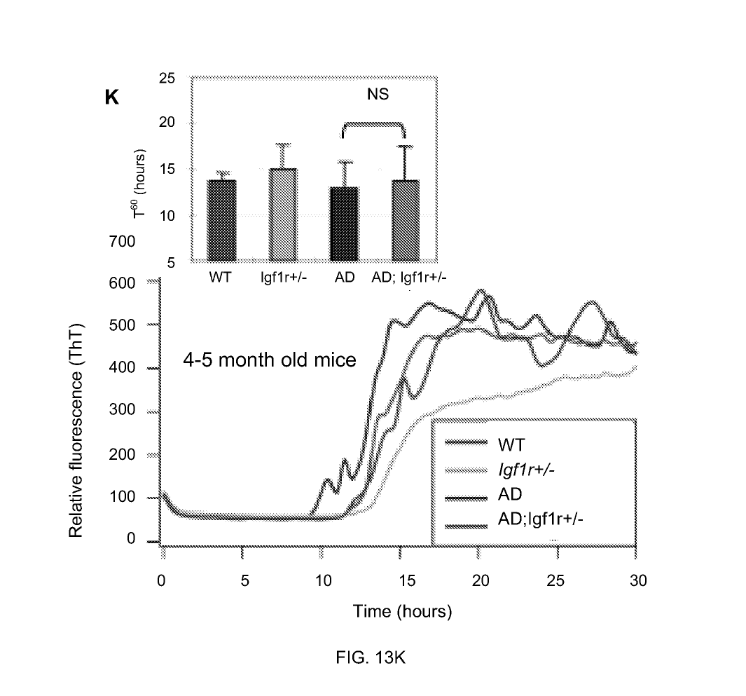

To further analyze the amyloid plaque density, we used postembedding immunoelectron microscopy, Aβ antibodies, and gold-labeled protein A. Aβ fibrils in the cortex of AD;Igf1r^{+/-} mice appeared to be more densely compacted than those of their AD counterparts (FIGS. 5A and 13A). (The lack of immunoreactivity in the brain sections of WT and Igf1r^{+/-} mice confirmed the specificity of the antibody; FIG. 13B).

To quantify and compare the density of the amyloid plaques of AD and AD;Igf1r^{+/-} mouse brains, we developed an electron microscopy (EM) image-processing algorithm that identifies the gold particles conjugated to the Aβ antibodies (FIG. 13C-F), sets a region of interest (ROI) around each particle, and determines the median signal density within the ROI after excluding the gold particle (FIG. 13G-I). ROIs that contain dense structures will have a lower score value due to less bright pixels and more dark pixels (i.e., lower gray-scale value). Cortices of six 12- to 13-month-old AD mice and five AD;Igf1r^{+/-} mice were visualized by EM and 135 images (34,087 ROIs) of AD and 101 images (26,066 ROIs) of AD;Igf1r^{+/-} were automatically segmented and analyzed in an unbiased manner. The distributions of ROI median signal intensities indicate that plaques of AD;Igf1r^{+/-} mice were significantly (p<0.038) denser than those of age-matched AD counterparts (FIG. 5B). The possibility that antibody accessibility to plaques of AD and AD;Igf1r^{+/-} brains differs was assessed by a second algorithm designed to measure the distance between each gold particle and its closest neighboring particle. This algorithm is based on the assumption that lower accessibility would result in sparse distribution and longer distances among the gold particles. Automatic processing of all plaque images of AD and AD;Igf1r^{+/-} showed no difference in distances (FIG. 13J, p>0.54), indicating similar antibody accessibilities.

The results obtained using light and electron microscopy suggest that reduced IGF signaling mediates the assembly of Aβ into more condensed amyloid plaques of lower toxicity. We used an in vitro kinetic aggregation assay (Cohen et al., 2006) to assess the relative total amounts of Aβ amyloid in equal volumes of brains of AD and AD;Igf1r^{+/-} mice. When proteinase K-treated and sonicated (fragments fibrils into a uniform size) brain homogenate is added to an Aβ₁₋₄₀ aggregation reaction, the reduction in the time that it takes the aggregation reaction to reach 50% completion is proportional to the amount of Aβ amyloid fibrils in the tissue (Cohen et al., 2006) (D.D. and J.K., unpublished data). The amyloid load was assessed in 4- to 5-month-old and of 12- to 13-month-old AD and AD;Igf1r^{+/-} mouse brain homogenates (nine animals per genotype). While no significant difference in aggregate load could be detected among brain homogenates of young animals (4-5 months old, FIG. 13K), brain extracts of 12- to 13-month-old AD;Igf1r^{+/-} mice exhibit a higher aggregate load reflected by a shorter t₅₀, (suggesting accelerated aggregation of Aβ₁₋₄₀) relative to age-matched AD animals (FIG. 5C; p=0.035). These data demonstrate that there is more amyloids in an equal volume of 12- to 13-month-old AD;Igf1r^{+/-} brain relative to AD brain. These results are consistent with the light and electron microscopy data indicating that protected AD;Igf1r^{+/-} animals have more densely packed Aβ aggregates than AD animals.

### Reduced IGF-1 Signaling Increases High-MW Aggregates and Reduces SDS-Soluble Aggregates

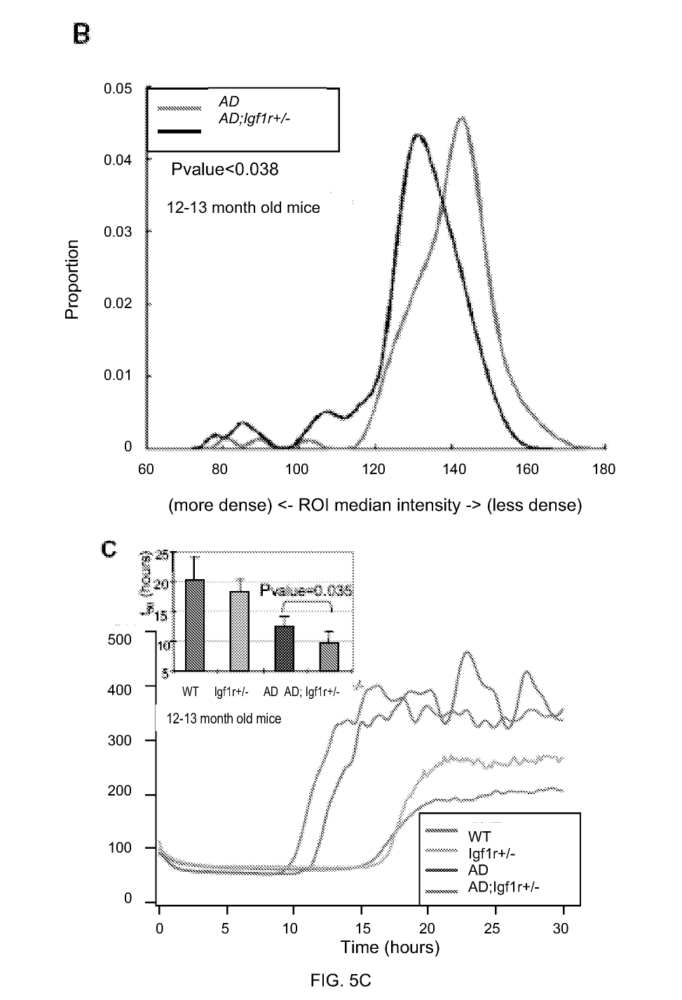

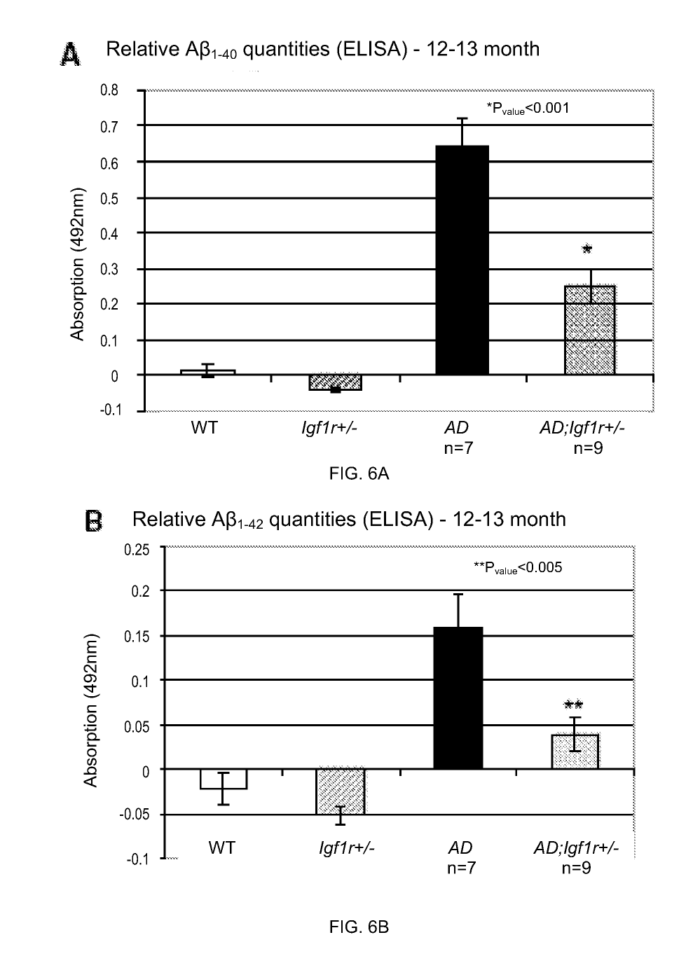

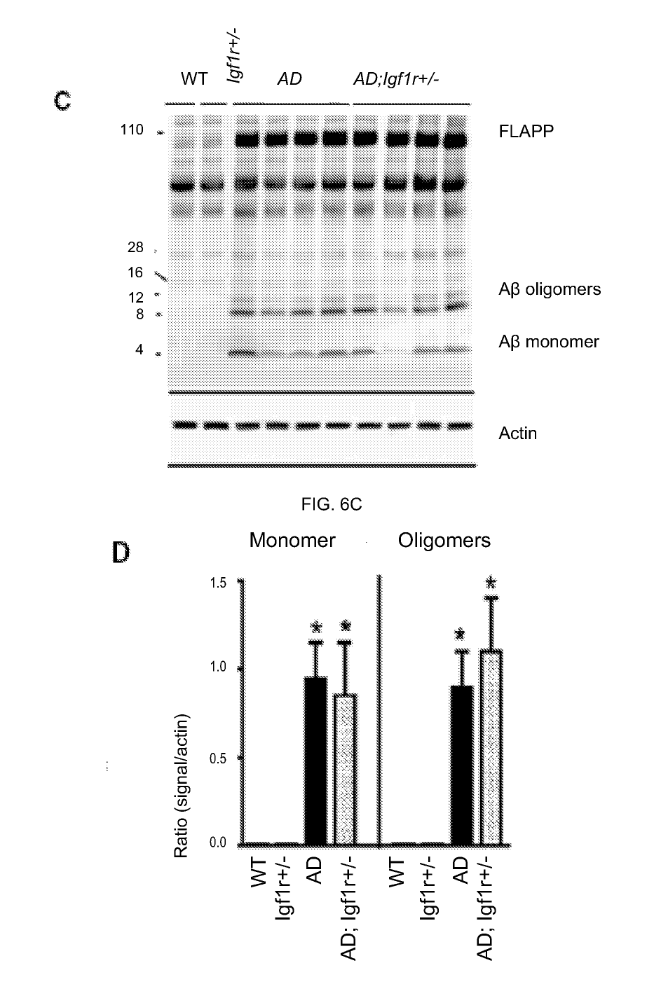

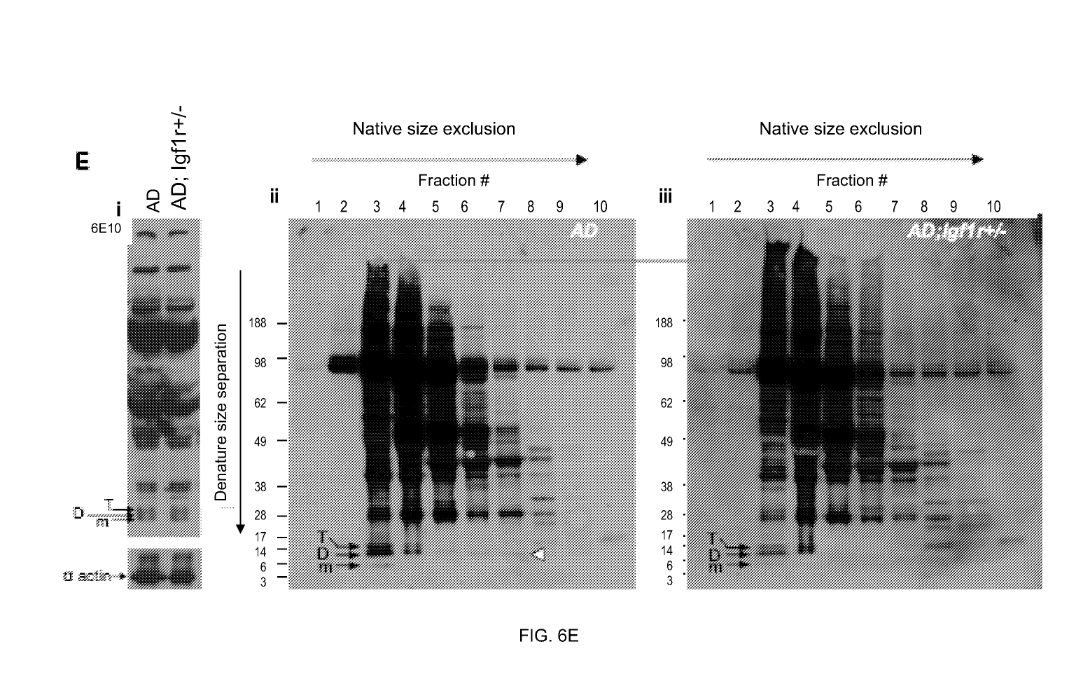

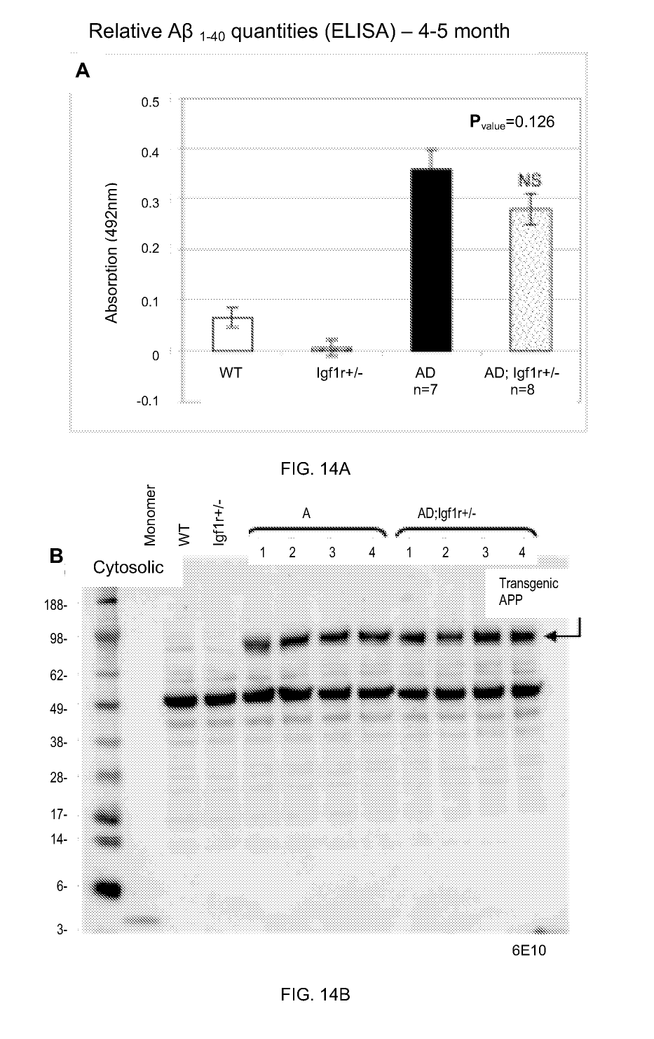

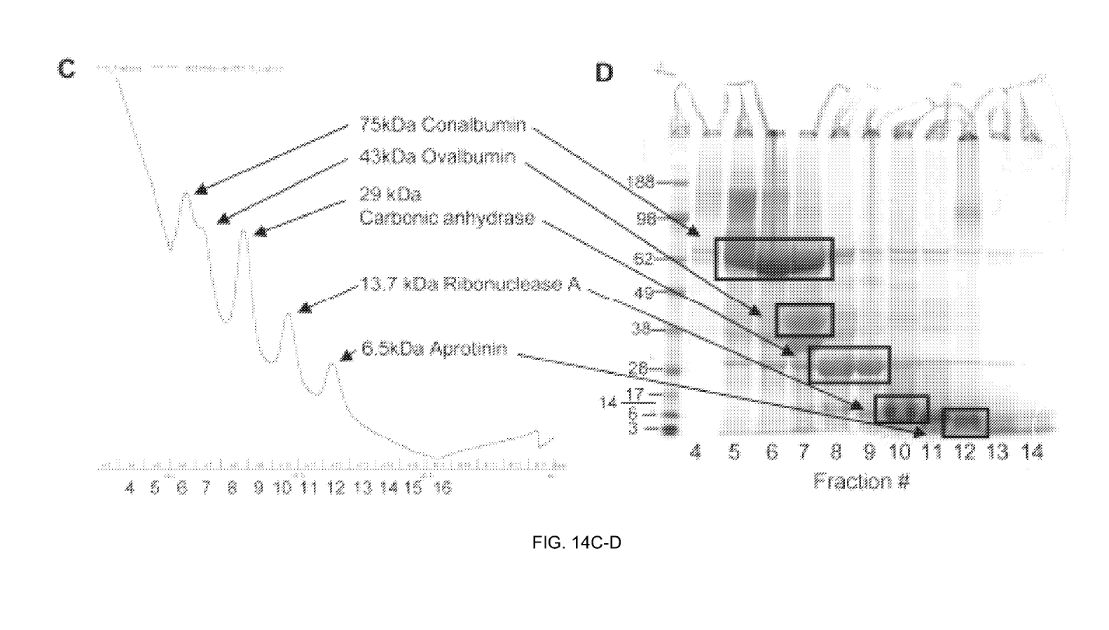

The hyperaggregation of Aβ by reduced IGF-1 signaling predicts lower residual amounts of nonaggregated Aβ and/or oligomeric Aβ. Thus, we tested whether more soluble Aβ is present in AD compared to AD;Igf1r^{+/-} brain homogenates. We spun brain homogenates of seven AD and nine AD;Igf1r^{+/-} 12- to 13-month-old mice to sediment highly aggregated Aβ (10,000 g for 10 min, 4° C.) and quantified the lower molecular weight (MW) Aβ₁₋₄₀ and Aβ₁₋₄₂ levels in the soluble fractions using enzyme-linked immunosorbent (ELISA) assays. Aβ₁₋₄₀ (FIG. 6A) and Aβ₁₋₄₂ (FIG. 6B) levels were significantly (p<0.001 and p<0.005, respectively) lower in the soluble brain supernatant fractions of AD;Igf1r^{+/-} mice compared to age-matched (12-13 month) AD animals. No such differences could be detected in the amounts of soluble Aβ₁₋₄₀ among young mice (FIG. 14A, p=0.126).

Next, we tested whether SDS-soluble Aβ oligomeric content and total quantities were affected by reduced IGF-1 signaling. Four AD and 4 AD;Igf1r^{+/-} mouse brains of 12- to 13-month-old mice were subjected to an Aβ oligomer preparation protocol (Bar-On et al., 2006) followed by SDS-PAGE and western blot (WB) analysis. Surprisingly, the SDS-soluble Aβ oligomer content, and total quantities and amounts of APP were indistinguishable in the total brain homogenates (FIGS. 6C and 6D) of AD and of AD;Igf1r^{+/-} mice (no oligomers could be detected in cytosolic fractions; FIG. 14B). The difference between the oligomer analysis results and the marked difference in the pool of nonaggregated Aβ species among AD;Igf1r^{+/-} and AD animals observed by the ELISA assays (FIGS. 6A and 6B) suggests that the oligomeric Aβ assemblies are SDS sensitive. To test this, we employed size-exclusion chromatography (SEC) to analyze the native composition of Aβ assemblies in the brains of AD and AD;Igf1r^{+/-} mice. Brains of AD;Igf1r^{+/-} and AD mice were homogenized and prepared as done for the ELISA assays to preserve macromolecular structural integrity. Equal amounts of cleared homogenates (FIG. 6E, panel i) were loaded onto a size-exclusion column, and 20 fractions were collected, lyophilized, resuspended, and loaded onto SDS gels. Aβ assemblies were visualized using WB and Aβ antibody (6E10). Notably, higher MW assemblies were observed in the AD;Igf1r^{+/-} (see red reference line) reflecting that they were larger to begin with and/or that they were more SDS resistant (FIG. 6E, panel iii, fraction 3). The apparent dimer band resulting from SDS mediated denaturation of much larger aggregates is not observable in the AD;Igf1r^{+/-} SEC fractions (FIG. 6E, panel iii), but is observable in the AD mouse fractions, (FIG. 6E, panel ii, fractions 5-7, open arrowhead). This proposes that in the AD;Igf1r^{+/-} brains, Aβ fibrils are denser, more SDS resistant and more efficiently prevent the release of potentially toxic oligomeric species. Since toxicity has been previously associated with the capacity of high-MW assemblies to fragment (Shankar et al., 2008), prior correlations between the appearance of small SDS-stabilized Aβ species and neurotoxicity may reflect this. The data obtained from the microscopic analyses, ELISA and in vitro assays suggest that the conversion of oligomers into denser, higher MW, more SDS-resistant aggregates is part of the process that protects against proteotoxicity in the AD;Igf1r^{+/-} animals.

### Discussion

By comparing behavioral and pathological aspects of Alzheimer's-like disease in the AD and AD;Igf1r^{+/-} mice, we found that reduced IGF-1 signaling notably protects mice from proteotoxicity associated with the expression of the AD-linked human peptide, Aβ. Light and electron microscopy, as well as in vitro kinetic aggregation, ELISA, and SEC assays, all indicate that reduced IGF-1 signaling induces the assembly of Aβ into densely packed, larger fibrillar structures late in life. The observation that the protected AD;Igf1r^{+/-} mice form SDS stable Aβ assemblies, making it more difficult to generate presumably toxic Aβ dimers (Shankar et al., 2008), suggests that an active mechanism converts oligomers into densely packed aggregates of lower toxicity that protect the AD;Igf1r^{+/-} mice from proteotoxicity. This hypothesis is consistent with results obtained in the Aβ worm model, where reduced insulin/IGF signaling protected worms from Aβ-associated toxicity while increasing the formation of high-MW Aβ aggregates (Cohen et al., 2006).

How can increased Aβ aggregation protect against proteotoxicity? Highly aggregated Aβ is thought to bear lower toxicity in comparison to oligomers (Haass and Selkoe, 2007). Accordingly, enhanced fibrillization can reduce Aβ toxicity in an AD-murine model (Cheng et al., 2007). Furthermore, results from long-term potentiation assays show that highly aggregated Aβ bears lower toxicity than small oligomers (Shankar et al., 2008). Intriguingly, the release of small oligomers, most notably dimers, from large Aβ assemblies (fibrils) by chemical extraction increases toxicity. In support of the hypothesis that accelerated aggregation can be protective is provided by the discoveries that the cellular chaperones HSP104 (Shorter and Lindquist, 2004) and TRiC (Behrends et al., 2006), both known to disrupt toxic protein aggregates can also mediate protection by accelerating aggregation when the concentration of the aggregating protein exceeded a threshold level. These studies raise the prospect that the creation of densely packed, large Aβ assemblies protects AD;Igf1r^{+/-} mice from proteotoxicity by trapping and storing highly toxic small aggregate structures. If active aggregation protects from Aβ toxicity, such protective mechanism might be expected to be negatively regulated by the IGF signaling pathway. In the worm, this activity is mediated, at least in part, by the FOXO transcription factor DAF-16 (Cohen et al., 2006), which is negatively regulated by the IIS receptor DAF-2. The FOXO gene family is highly conserved in mammals, is expressed in neurons, and is required for neuronal survival under stress (Lehtinen et al., 2006), suggesting that FOXO transcription factors are also mediators of the reduced IGF signaling protective effect in mammals.

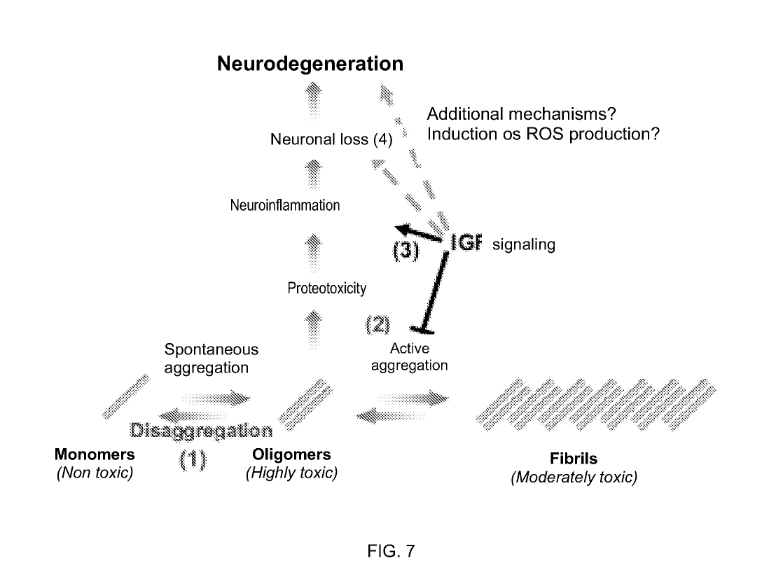

It is likely that reduced IGF signaling ameliorates Aβ proteotoxicity by mechanisms in addition to Aβ dense fibril formation. The observation that Igf1r+/− mice exhibit increased resistance to oxidative stress (Holzenberger et al., 2003) raises the possibility that reduced IGF-1 signaling enhances the neuronal counter proteotoxic capabilities by enhancing the levels of enzymes that protect against oxidative stress proposed to be involved in AD-associated brain damage (Fukui et al., 2007). This is supported by the observation that the production of reactive oxygen species is reduced in brains of Igf1r+/− mice compared with their WT counterparts following MPTP treatment known to induce a Parkinson's disease-like phenotype (Nadjar et al., 2008). Moreover, overexpression of mitochondrial-targeted catalase promotes longevity of mice (Schriner et al., 2005). An alternative model suggests that increased neuronal resilience associated with reduced IGF signaling is promoted by enhanced DNA repair capabilities. It is reasonable to speculate that the histone deacetylase SIRT1, an aging regulator (Ghosh, 2008) that plays roles in the maintenance of genomic stability (Oberdoerffer et al., 2008) and regulates HSF-1 (Westerheide et al., 2009), may also be a mediator of the reduced IGF signaling protective effect in the AD;Igf1r^{+/-} mice. The complexity and variety of effects mediated by FOXO (Partridge and Bruning, 2008) propose that reduced IGF signaling orchestrates an array of counter proteotoxic activities including Aβ hyperaggregation, counter oxidation activities, and presumably other yet to be defined mechanisms (FIG. 7). Further research is required to elucidate whether mammalian FOXO family members play roles in the protective mechanism toward AD.

The Aβ hyperaggregation observed in protected AD;Igf1r^{+/-} mouse brains suggested that Aβ plaques would be visible in the cortex of these animals at younger ages compared to their unprotected AD counterparts, however, this was not evident in our analysis. This is likely due to other mechanisms of protein homeostasis being effective early in life, such as the disaggregase and degradation activity regulated by HSF-1, as observed in the worm (Cohen et al., 2006). In this view, the protective disaggregation/degradation and hyperaggregation mechanisms may be temporally distinct. Active hyperaggregation may only be invoked once the primary disaggregation machinery can no longer effectively clear toxic Aβ species as a consequence of aging or an extrinsic stress. It will be interesting to evaluate whether one or more of the four HSF genes in the mouse are involved in protecting the brain from Aβ toxicity throughout life, and whether FOXO activity becomes prominent later in life.

There is an apparent contradiction between the data presented herein and earlier reports that IGF infusion protects rats (Carro et al., 2002) and mice (Carro et al., 2006) from Aβ proteotoxicity and that IGF-1R blockade induced neurological disease in rats (Carro et al., 2006). The presence of feedback-signaling events that respond to the sudden increase in IGF concentration by tuning down the responsiveness of the IGF signaling cascade over time could explain why IGF infusion is protective against AD-like pathology (Cohen and Dillin, 2008). This explanation is supported by many observations. For example, long-lived female human centenarians have high serum IGF-1 levels, but low IGF-1R activity, leading to reduced IGF signaling (Suh et al., 2008). Therefore, high IGF-1 levels do not necessarily correlate with increased downstream activity over a prolonged time. Additionally, AD patients have lower than normal serum insulin concentrations, but higher than normal CSF insulin levels (Craft et al., 1998). These studies raise the prospect that insulin and IGF signaling are regulated in a tissue-specific manner, and suggest that peripheral IGF infusion may lead to reduced IGF signaling in the brain (Cohen and Dillin, 2008).

The data presented here demonstrate that reduced IGF-1R signaling results in a profound reduction in the toxicity associated with Aβ expression in the brains of mice. The formation of larger and denser Aβ aggregates that appear to be more SDS resistant in the AD;Igf1r^{+/-} mice suggests that this is one core protective activity regulated at least in part by IGF-1R signaling, much like the disaggregase activity reported previously (Cohen et al., 2006). The indication that reduced IIS is protective in nematodes and mammals stresses that manipulation of the highly conserved IGF signaling pathway, and its downstream components, is promising for the development of novel neurodegeneration and proteotoxicity therapies.

### Supplemental Data

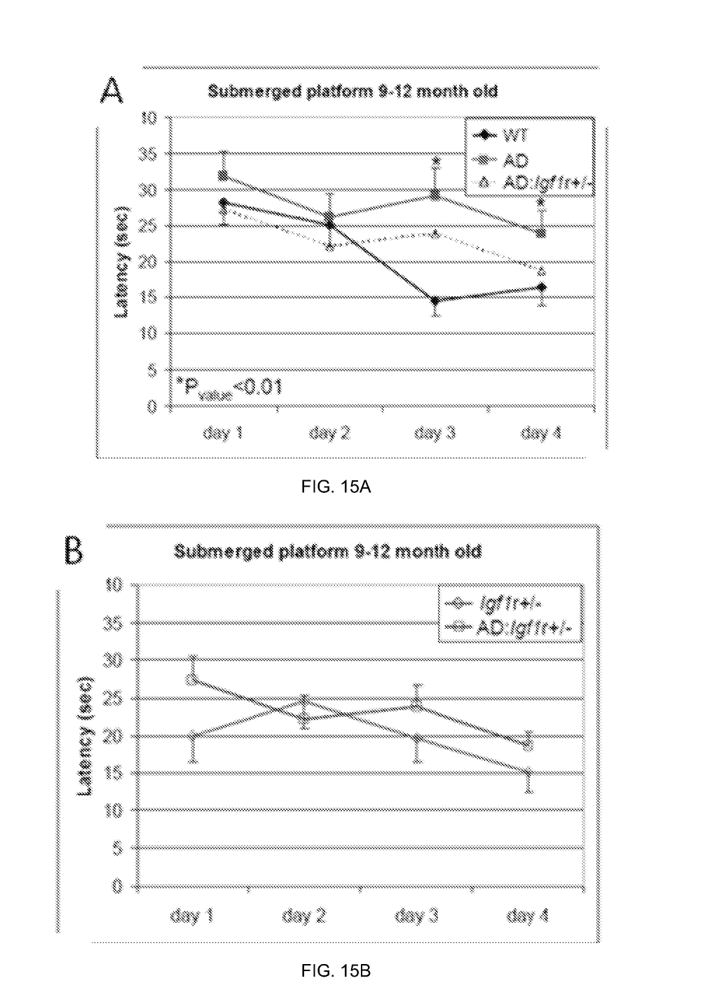

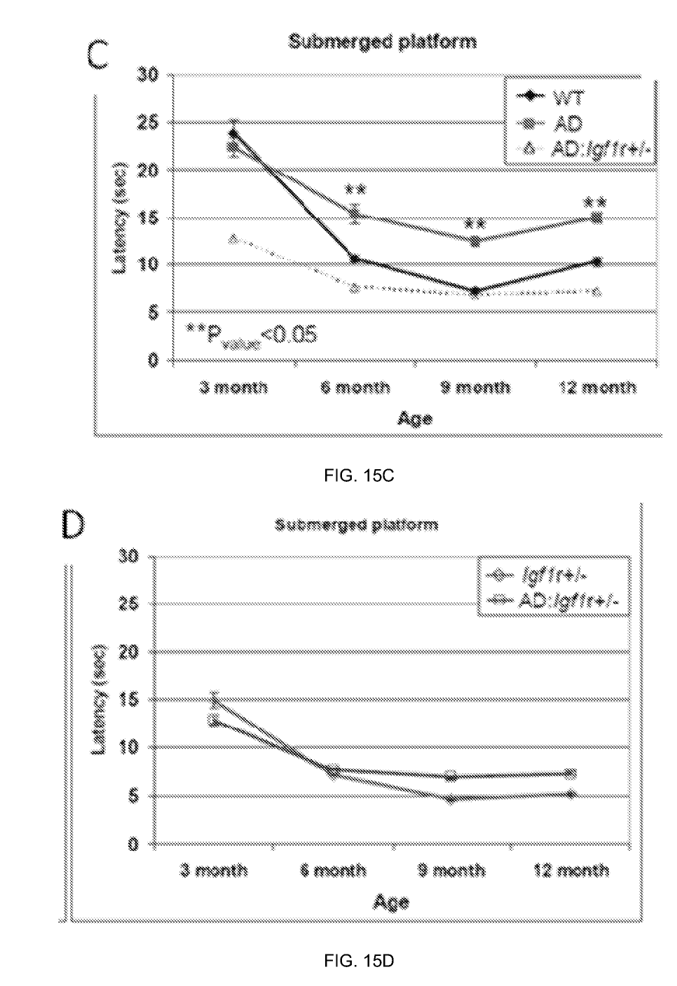

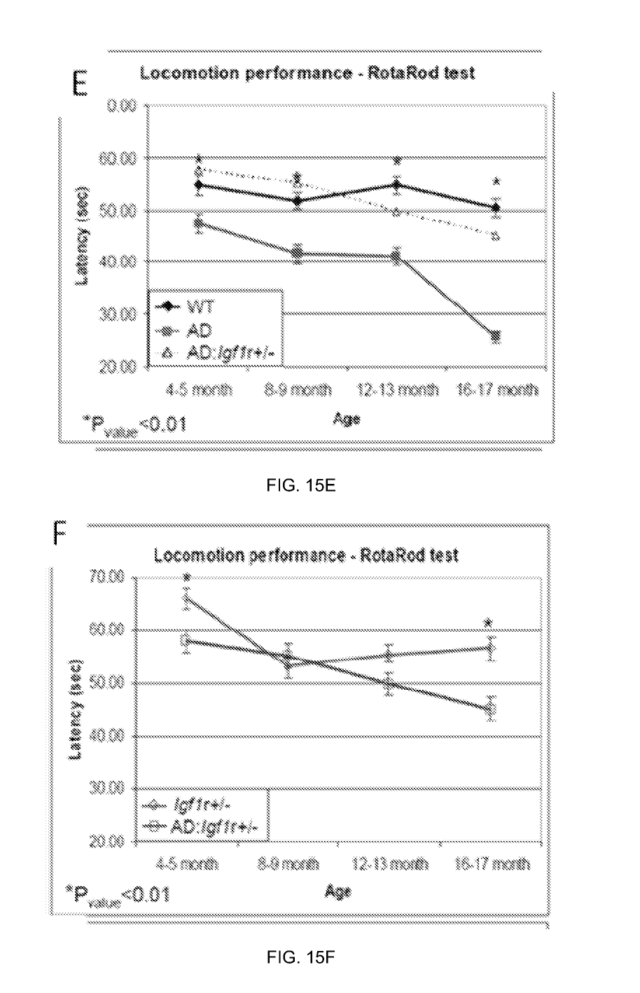

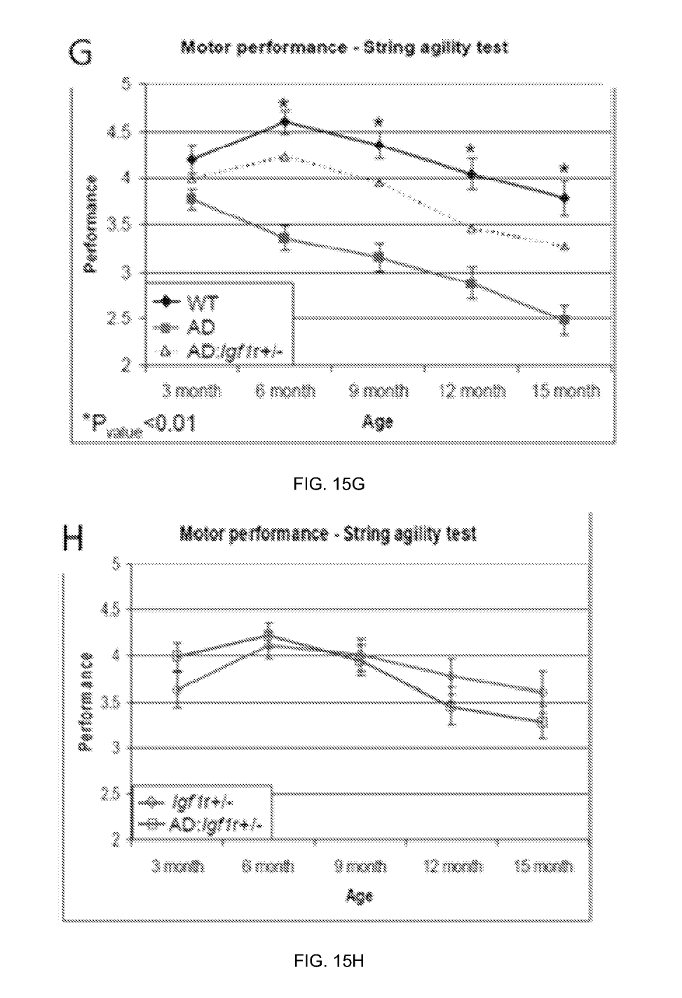

Because the ability of the animals to remember the position of a submerged hidden platform is reduced in AD model mice, memory and orientation aptitude was assessed by removing the flag from the platform and recording the latency time required for animals to find the submerged hidden platform. At days 3 and 4 of the experiment, 9-12 month old AD mice required a significantly (Pᵥₐₗᵤₑ<0.01) longer time to find the hidden platform compared to their WT counterparts (FIG. 15A). In contrast, AD:Igf1r+/− mice found the hidden platform at nearly the same average time as their Igf1r+/− and WT counterparts throughout the experiment (FIG. 15B) (swim speeds were nearly identical for all genotypes). We also tested independent cohorts of mice throughout 12 months of age. AD and WT mice performed similarly at 3 month of age, however memory impairment (Pᵥₐₗᵤₑ<0.05) began at 6 months of age and became progressively worse at 9 and 12 months (FIG. 15C). Consistent with our 9-12 month data, we found that reduction of IGF1 signaling largely restored memory at all ages (FIG. 1D). Consistent with our 9-12 month data, we found that reduction of IGF1 signaling largely restored memory at all ages (FIG. 1E).

Next, we tested the effect of reduced IGF-1 signalling on the motor skills of AD model mice using both Rota-Rod assay to measure locomotion, and the string agility assay to measure motor coordination. Much like the memory test, the AD mice did not perform as well as their WT counterparts in both the Rota-Rod (FIG. 15F) and string agility tests (FIG. 15H). In contrast, AD:Igf1r+/− mice performed nearly as well as their Igf1r+/− and WT counterparts, exhibiting insignificant differences in both assays (FIG. 15F-I)), Collective consideration of the behavioral data reveal that AD mice have normal learning, but impaired memory and this decline can be delayed by protection conferred by reduced IGF-1 signaling. Similarly, locomotion and coordination impairments associated with the human AD-linked transgenes were greatly delayed by reduced IGF-1 signaling.

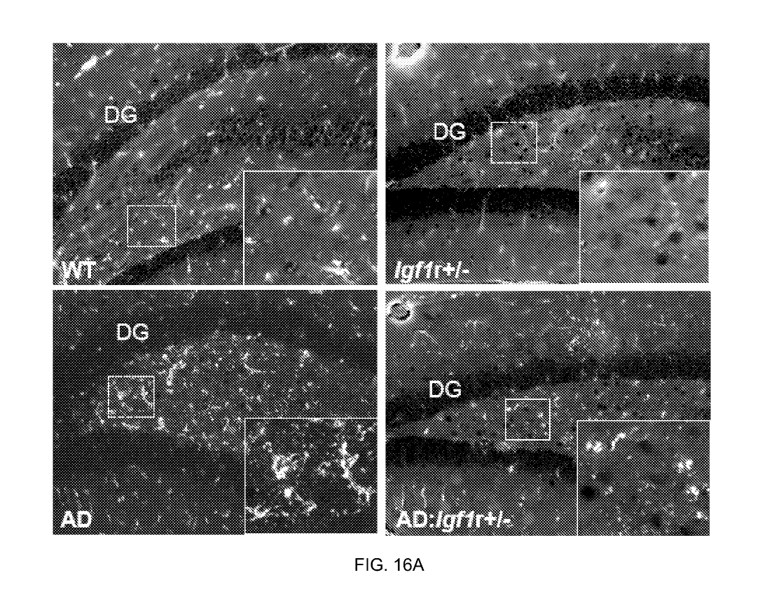

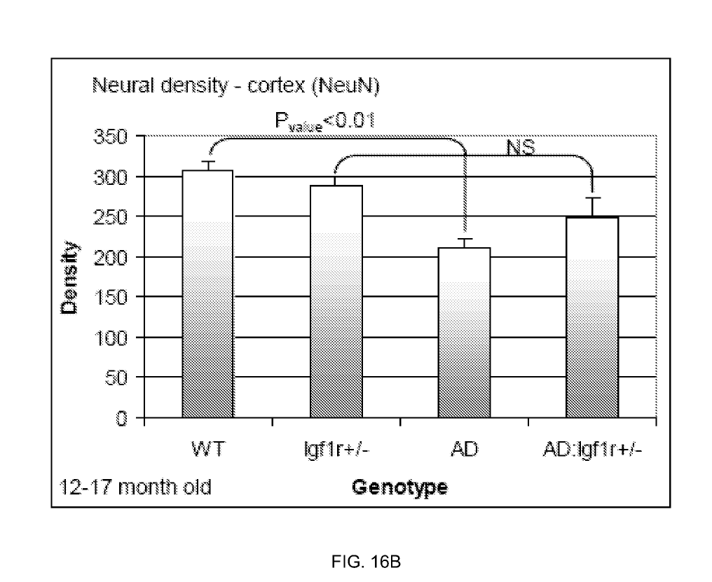

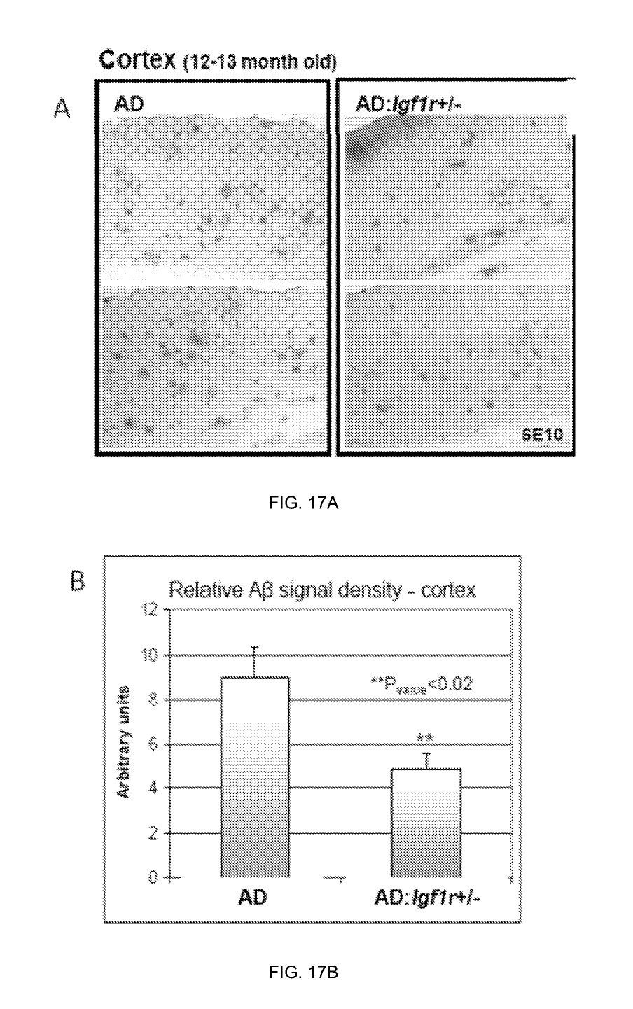

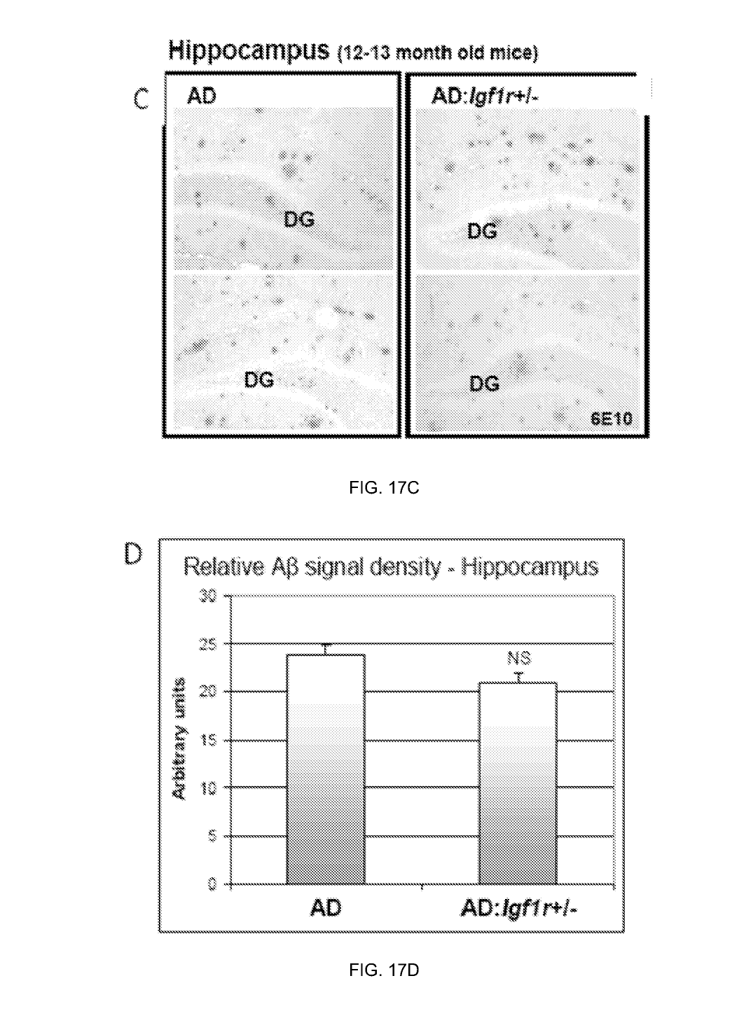

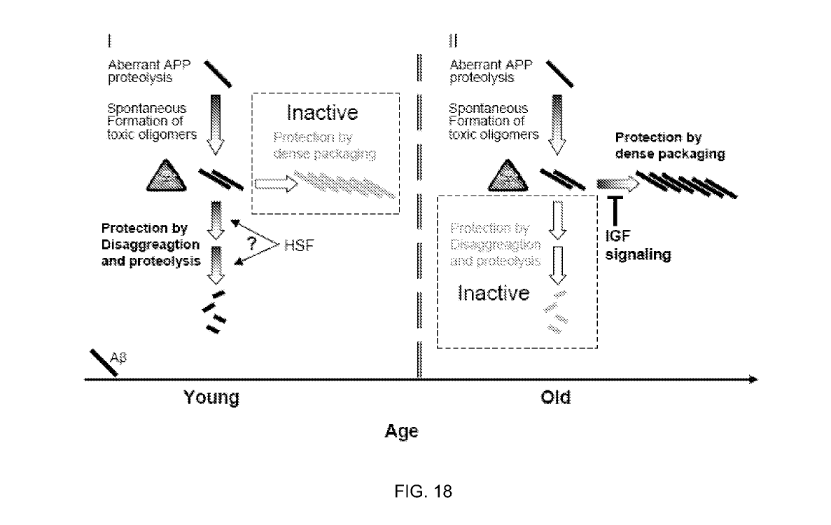

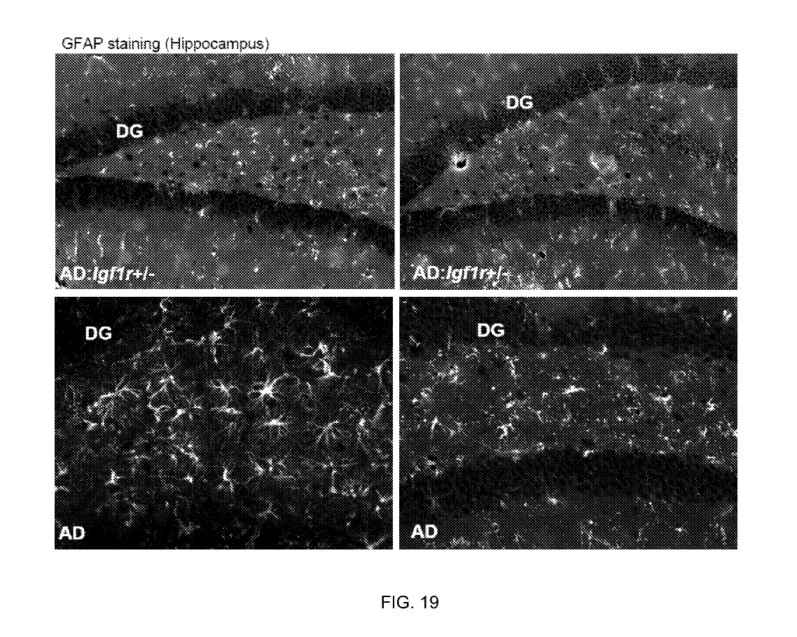

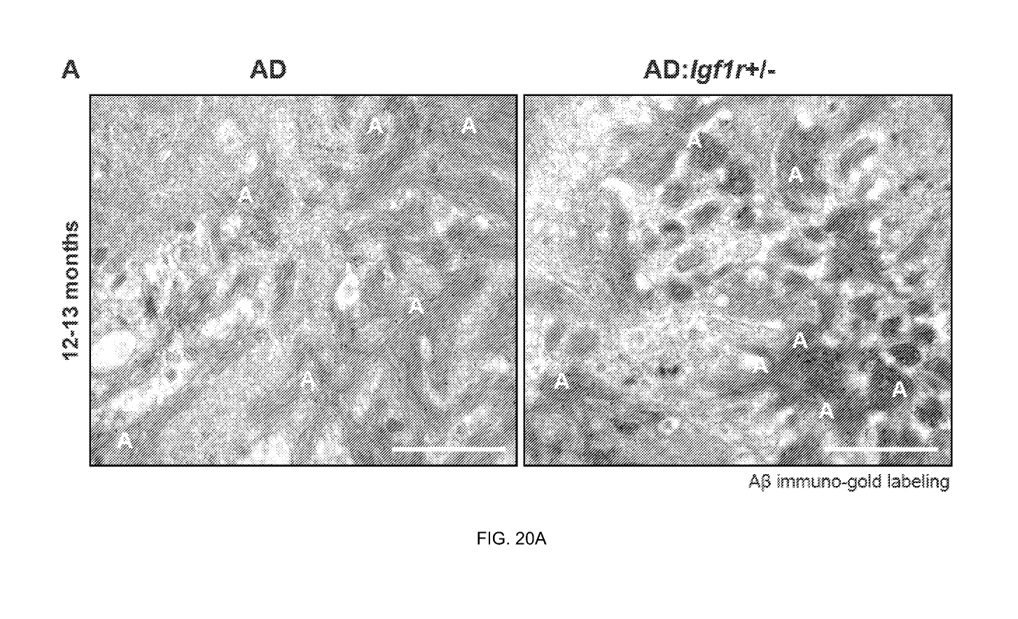

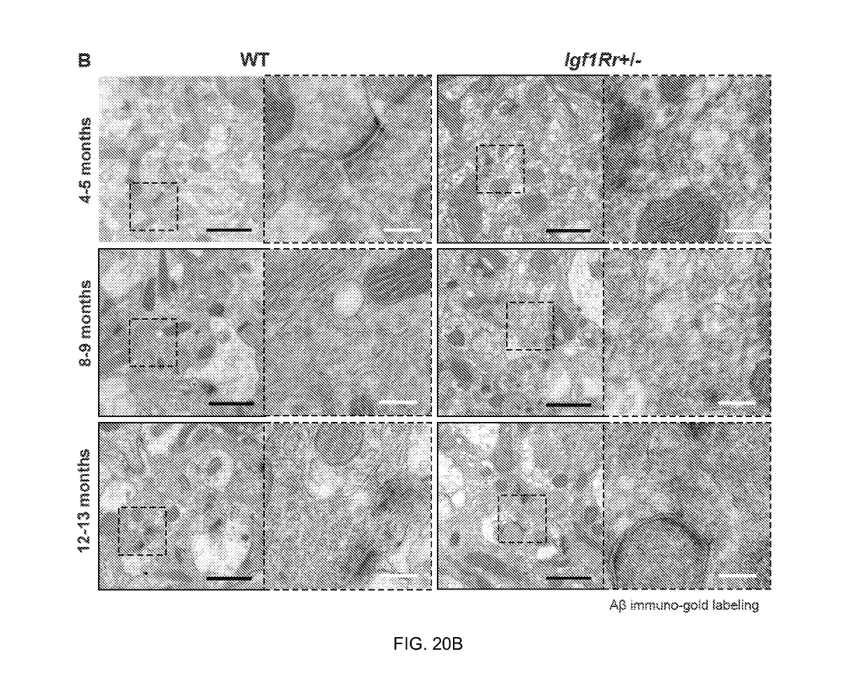

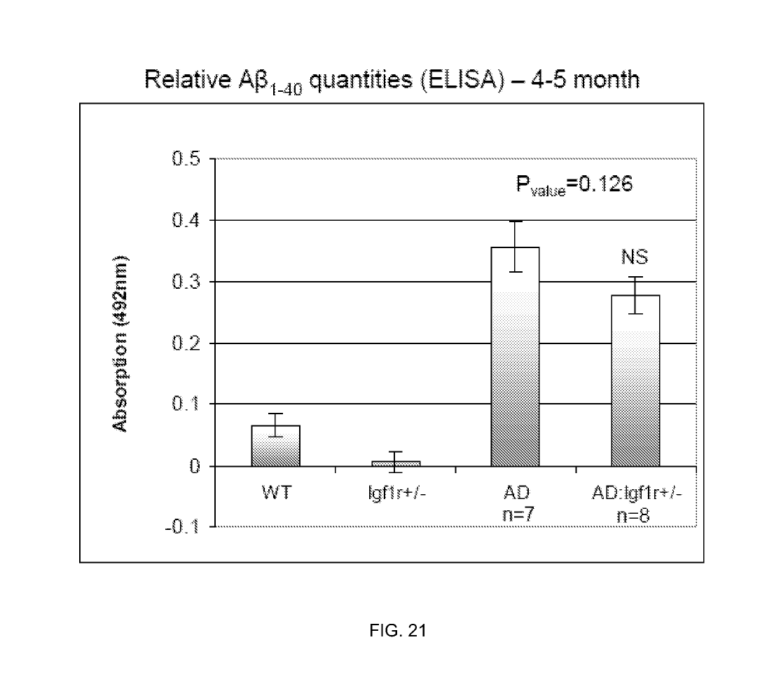

As discussed above, Aβ plaques observed in the cortex of protected AD:Igf1r+/− animals appeared to be smaller in size and more condensed than plaques detected in the cortex of their age matched unprotected AD counterparts. Signal densitometry image analysis confirmed the more dense content of plaques in the cortex (FIGS. 17A and B), but not in the hippocampus (FIGS. 17C and D).

### Experimental Procedures

### Mouse Strains and Genotyping

AD-model male mouse expressing a mutant chimeric mouse/human APP_{swe} and a mutant human presenilin 1 (Delta E9) both driven by the prion protein promoter was purchased from Jackson laboratory (strain B6C3-Tg [APP_{swe} PSEN1 dE9] 85 Dbo/J, stock number 004462).

Long-Lived, Compromised IIS Mice

Males harboring only one Igf1r copy (S129 background [Holzenberger et al., 2003]) were obtained from Dr. Jeffery Friedman (TSRI, La Jolla, Calif.). Males of both strains were crossed for three generations with “wild-type” 129 females (Jackson laboratories, strain 129Xi/SvJ, stock number 000691), to set up two separate colonies. Mice of each colony were backcrossed for additional two generations. Next, Igf1r^{+/-} males were crossed with AD females for three generations to generate the experimental mice.

DNA was purified from biopsies of mouse tails and subjected to PCR. APP_{swe} and PS1ΔE9 were amplified as directed by the Jackson Laboratories. Igf1r was amplified using primers.

### Western Blot Analysis

Brains were dissected, homogenized, and divided by ultracentrifugation (100,000 g, 1 hr, 4° C.) into cytosolic and membrane (particulate) fractions. For WB analysis, 15 μg per lane of cytosolic and particulate fractions, assayed by the Lowry method, were loaded into 10% SDS-PAGE gels and blotted onto nitrocellulose paper. Blots were incubated O/N with antibodies against APP/Aβ (6E10), Aβ (82E1), and C terminus APP(CT-15, courtesy of Dr. Ed Koo). Next, membranes were incubated with secondary antibodies tagged with horseradish peroxidase (1:5000, Santa Cruz Biotechnology, Inc., Santa Cruz, Calif.), visualized by enhanced chemiluminescence and analyzed with a Versadoc XL imaging apparatus (Bio-Rad, Hercules, Calif.). Actin served as a loading control.

### Rota Rod

Locomotion was tested using Rota Rod system (EconoMex, Columbus Instruments, Columbus, Ohio). Four mice were place at a time on the rotating beam set to accelerate at 0.2 rpm/s.

Time from start until each mouse fell off was recorded. Each mouse was trained 1 day for five times prior to the experiment. Each mouse was tested five times a day, for 4 sequential days (total of 20 measurements/mouse/age). At least 12 animals (males and females) per genotype were used in each time point (Supplemental Statistical Data).

### Morris Water Maze

The Morris water maze was conducted as described previously (Jensen et al., 2005). Briefly, 11- to 15-month-old mice of the four genotypes were placed one animal per cage and numbered randomly to avoid genotype identification during the experiment. A plastic tank 120 cm in diameter was filled with room temperature (RT) water (23° C.), which was made opaque with white nontoxic paint. A transparent platform (8 cm×12 cm) was located in the center of one of the four virtually divided quadrants and was submerged 0.5 cm below the water surface to be invisible. Distal cues were provided in all experiments as spatial references. Mice were let swim until platform was found or for a maximum of 60 s. Mice were allowed to rest on the platform for 15 s between trials. In all experimental settings we utilized a video tracking system (Ethovision; Noldus Information Technology, Leesburg, Va.) to record and analyze the swimming path, swim velocity, time taken to reach the platform (latency), and time spent in each quadrant. The experiments were performed at the following order: cued platform (4 sequential days), hidden platform (4 sequential days), probe trial (1 day). The number of animals used was 13 (WT), 12 (Igf1r^{+/-}), 8 (AD), and 15 (AD;Igf1r^{+/-}).

### Cued Platform

For the cued version of water maze testing, the platform was located 0.5 cm below the opaque water level but made clearly visible to the mouse by locating a 15 cm high stick carrying a dotted flag (3 cm×4 cm) on the platform. The platform location was fixed throughout the experiment. The mice were released from four different locations around the water tank.

### Hidden Platform

The platform was located at the same location used for the cued platform experiment, 0.5 cm below the opaque water level but without the dotted flag, to be invisible. The mice were released from four different locations around the tank. Time of latency, swim velocity, path length, and time spent at each quadrant were recorded.

### Probe Trial

The platform was removed and the mice were allowed to swim for 40 s. The time spent in each quadrant and in the previous platform location, number of crossing the area where the platform was previously located, swim velocity, and path length were recorded.

### Size-Exclusion Chromatography

A Superdex 75 10/300 GL column (Cat #17-5174-01 GE Healthcare, Uppsala Sweden) attached to an AKTA FPLC system was used to separate Aβ oligomers from mouse brains. Column was calibrated using low MW calibration kit (GE Healthcare cat #28-4038-41). Then 250 μl 10% (w/v) mouse brain homogenate (in PBS) was injected into the column and eluted with 50 mM ammonium acetate (pH 8.5) at flow rate of 0.5 ml/min. Twenty 1 ml fractions were collected, lyophilized, resuspended in 120 μl PBS and 40 μl LDS sample buffer, boiled for 10 min, and separated on 4%-12% Bis-Tris gels as described above.

### Morphological and Postembedding Immunoelectron Microscopy

WT, Igf1r^{+/-}, AD, and AD;Igf1r^{+/-} mice were sacrificed at the indicated ages. A piece of cortex from each mouse brain was fixed for 24 hr in cold 2% paraformaldehyde and 0.25% glutaraldehyde in PBS followed by washing in PBS and postfixed in 1% osmium tetroxide in PBS. The samples were washed in PBS and dehydrated in graded ethanol solutions followed by propylene oxide and embedded in Epon/Araldite mixture (Cat #13940, Electron Microscopy Sciences, Hatfield, Pa.). The polymerized resin was sectioned (70 nm) using a diamond knife (Diatome, Hatfield, Pa.) and mounted on uncoated 400 mesh nickel grids (Cat# G400-Ni, Electron Microscopy Sciences) for immunolabeling. Antigen retrieval was performed using sodium m-periodate-saturated aqueous solution for 10 min followed by TBS (50 mmol/l Tris-HCl, 150 mmol/l NaCl [pH 7.4]) wash. Sections were background blocked in 3% bovine serum albumin (BSA) in TBS for 30 min followed by an overnight incubation in primary Aβ₁₋₄₂ affinity purified polyclonal rabbit antibody, which recognizes the C terminus of the peptide (Cat # AB5078P Chemicon-Millipore, Temecula, Calif.) 1:50 in 1% BSA in TBS at RT. Sections were washed 3 times in TBS and blocked in 3% BSA in TBS for 30 min followed by 2 hr incubation in protein A conjugated to 10 nm gold particles (Cat # EM PAG10 BB International, Cardiff, UK) diluted 1:100 in 1% BSA in TBS at RT, rinsed three times in TBS, three times in H₂O, and air dried. Higher contrast was achieved with 2% uranyl acetate in 50% ethanol for 10 min and in Reynold's lead citrate solution (120 mmol/l sodium citrate, 25 mmol/l lead citrate [pH 12]) for 1.5 min. The specimens were studied in a Jeol 100CX electron microscope (Jeol, Akishima, Tokyo, Japan) at 100 kV. Electron micrographs were taken with a Mega View III CCD camera (Soft Imaging System GmbH, Muenster, Germany) and Analysis Pro v 3.2 digital micrograph software (Soft Imaging System GmbH).

### In Vitro Kinetic Aβ Aggregation Assay

Aβ₁₋₄₀ peptide (10 μM in phosphate buffer: 300 mM NaCl, 50 mM Na-phosphate [pH 7.4]) was labeled with ThT (20 μM). Mouse brain homogenate was sonicated for 40 min (FS60, Fisher Scientific, Pittsburg, Pa.), treated with proteinase K (2 h, 0.2 μg/ml), and supplemented with complete EDTA-free protease inhibitor cocktail (cat#1836170 Roche, Basel Switzerland). Three aliquots (100 μl each, total protein concentration of 10 μg/ml) were transferred into a 96-well microplate (Costar black, clear bottom) for each reaction. The plate was loaded into a Gemini SpectraMax EM fluorescence plate reader (Molecular Devices, Sunnyvale, Calif.), incubated at 37° C., and fluorescence (excitation at 440 nm, emission at 485 nm) was measured from the bottom at 10 min intervals, with 5 s of shaking before each reading. Half-maximal fluorescence time points (t₅₀) were defined as the time point at which ThT fluorescence reached the middle between pre- and postaggregation baselines. Fluorescence traces and t₅₀ values represent averages of at least three independent experiments.

### RNA Isolation and Quantitative RT-PCR

Brains were removed from mice and flash froze. About 30 mg of tissue was cut from each forebrain and transferred into 500 μl Qiazol (Cat #79306 Qiagen, Hilden Germany), homogenized using a syringe and needle and froze at −80° C. for 4 hours. The samples were thawed, supplemented with 120 μl chloroform and spun for 10 min (18,000 g). Total RNA was isolated from the supernatants using RNeasy kit (Qiagen cat #74106). cDNA was prepared from 1 μg RNA using Quanti Tect kit (Qiagen cat #204341). For quantitative PCR reactions, dilutions of 10× were used. SybrGreen real-time qPCR experiments were performed as described in the manual using ABI Prism7900HT (Applied Biosystems). Quantification was completed using SDS2.1 software (Applied Biosystems), normalizing to control levels of β-actin cDNA.

### Aβ Blotting and Detection

### Aβ Oligomer Analysis

A posterior half hemisphere (approximately 100 mg) was taken from each mouse brain, supplemented with 700 μl PDGF buffer (1 mM HEPES, 5 mM Benzamidine, 2 mM β-Mercaptoethanol, 3 mM EDTA, 5 mM Magnesium Sulfate, 0.05% Sodium Azide, pH8.8) and phosphatase and protease inhibitor cocktails (Calbiochem, San Diego, Calif. cat #524625 and 539134 respectively). The brains were sonicated and spun (5 min, 5000 rpm, desktop centrifuge). Pellets were supplemented with 500 μl PDGF buffer and sonicated again (Total). Supernatants were spun for 1 h, 100,000 rpm, 4° C. (Beckman TL-100 desktop ultracentrifuge, rotor TL-120.2, gav=355,000 g). Ultracentrifugation supernatants were transferred to new tubes (Cytosolic) while pellets were resuspended in 300 μl PDGF buffer (Particulate). BCA kit was used to measure total protein amounts. Equal total protein amounts were separated on 4-12% Bis-Tris gels (Invitrogen, Carlsbad, Calif. cat #NP0322), transferred onto nitrocellulose membrane (Protean 0.2 μm Whatman, Dassel Germany) and blotted with 6E10 Aβ antibody (SIG-39320 Covance Emeryville, Calif.). ECL was developed using Lumi-light plus kit (Roche, Basel Switzerland). The levels of BACE were measured using Western blost (ProSci Inc., CA), ADAM17 (TACE) was detected using a monoclonal specific antibody (Abcam, Mass.) and actin was blotted using Mab1501 antibody (Millipore, Mass.).

### Immunohistochemistry (IHC)

### Tissue Processing

Brains were removed and divided sagitally. The left hemibrain was post-fixed in phosphate-buffered 4% paraformaldehyde (pH 7.4) at 4° C. for 48 hrs and sectioned at 40 μm with a Vibratome 2000 (Leica, Germany), while the right hemibrain was snap frozen and stored at −70° C. for protein analysis.

### Analysis of Neurodegeneration and Aβ Deposits

To evaluate neurodegeneration, blind-coded 40 um thick vibratome sections were immunolabeled as previously described with monoclonal antibodies against NeuN (general neuronal marker, 1:1000, Chemicon) of GFAP (astroglial marker, 1:500, Chemicon) or synaptophysin (Millipore, MAB5258) and reacted with diamine-benzidine (DAB). The sections immunostained with anti-Neu were analyzed with the Stereo-Investigator Software (MBF Biosciences). Images collected according to the optical detector method were analyzed as previously described (Chana et al. 2003). For the sections immunostained with anti-GFAP, tissues were imaged with an Olympus digital microscope (BX51) and images analyzed with the ImageQuant program to determine levels of corrected optical density. Three immunolabeled sections were analyzed per mouse and the average of individual measurements was used to calculate group means. Results were expressed as the average number of GFAP+ cells per 10⁵ mm².

Aβ deposits were detected as previously described, briefly vibratome sections were incubated overnight at 4° C. with the mouse monoclonal antibody 4G8 (1:600, Senetek, Napa, Calif.), followed by incubation with a fluoroscein isothiocyanate (FITC)-conjugated anti-mouse IgG (Vector Laboratories). Sections were imaged with the LSCM (MRC 1024, BioRad) as described previously (Mucke et al. 2000) and digital images were analyzed with NIH Image 1.43 program to determine the percent area occupied by Aβ deposits. Three immunolabeled sections were analyzed per mouse and the average of individual measurements was used to calculate group means.

For analysis of the density of the Aβ deposits, sections were immunolabeled as previously described (Rockenstein et al., 2007) with the antibodies against Aβ (82E1 clone, prepared against Aβ 1-16) and reacted with DAB. From each case, 3 serial blind coded sections were scanned with a digital bright field photo-microscope (Olympus, BX51). From each section, 4 images of the neocortex and hippocampus were obtained an analyzed for levels of optical density with the ImageQuant program. Results were averaged and expressed as means per case. When Proteinase K (PK) treatment was applied, the sections were incubated with 10 ug/ml PK for 8 minutes prior to immunolabeling with specific Aβ antibody (82E1).

All sections were processed simultaneously under the same conditions and experiments were performed twice to assess reproducibility. Sections were imaged with an Olympus 60X (NA 1.4) objective on a digital Olympus or a Zeiss 63X (NA 1.4) objective on an Axiovert 35 microscope (Zeiss, Germany) with an attached MRC1024 LSCM system (BioRad) (Masliah et al., 2000). To confirm the specific primary antibodies, control experiments were performed where sections were incubated overnight in the absence of primary antibody (deleted) or preimmune serum and primary antibody alone.

### Aβ ELISA Assay

Mouse brains were removed and cut mid saggitaly. One hemisphere was homogenized in cold PBS containing complete protease inhibitor cocktail (Roche Cat#1836170) using a glass tissue grinder (885482, Kontes, Vineland, N.J.) to final concentration of 10% w/v. The homogenates were spun briefly to sediment debris (5000 rpm, 3 min, desktop centrifuge). Supernatants were transferred to new tubes and were spun again to sediment non-highly aggregated Aβ (rcf=10,000 g, 10 min, 4° C.). ELISA kits were used according to the manufacturer instructions to measure Aβ₁₋₄₀ and Aβ₁₋₄₂ contents in the secondary supernatants (Cat# SIG-38940 and SIG-38942, respectively, Covance, Emeryville, Calif.). 9 ul of each supernatant were used for Aβ₁₋₄₀ assay and 72 ul of each supernatant to measure Aβ₁₋₄₂.

### Statistical Methods

Data analysis was performed using parametric linear models (one-way or two-way analysis of variance) or their counterpart non-parametric substitutes for cases where the data was not normally distributed. Post-hoc pair-wise contrasts (planned comparisons) were obtained by Fisher-Least Square Differences (LSD). In the current study, this need raised for the analysis of the “probe trials” in the Morris water maze and the comparison of EM particle intensity. For this purpose, we analyzed the corresponding data using the Kruskal-Wallis non-parametric test followed by post hoc Mann-Whitney U test. In all cases, significance was considered for p-values lower than 0.05. Normal distribution was tested using a two-sided Lilliefors' composite goodness-of-fit test with the null hypothesis that data is normally distributed. All data analysis was performed under Matlab (Mathworks Inc.) by implementing the provided statistical toolbox. Neuronal counts (NeuN-based) datasets of both cortex and hippocampal areas were analyzed for each age group separately using a one-way ANOVA design. Only the hippocampal counts for the 12-13 month age group was analyzed using the Kruskall-Wallis non-parametric test as it did not fulfill the Lilliefor normality test. Post-hoc comparisons were performed via Tukey-Kramer test. In all analyses, a confidence level of 95% was used, for all age groups, the degrees of freedom are the same (i.e., df=3) and the number of cases per genotypes are presented in the following order: WT, IgfR1+/1, AD and AD:IgfR1+/−.

### EM Particle Analysis

We have developed an unsupervised algorithm for automatic densitometry analysis of gold-labeled electron microscope images. The algorithm consists of two major steps: i) gold particle segmentation via adaptive thresholding; and ii) gold particle removal prior to intensity measurement on a region of interest (ROI) located around each labeled particle. For the segmentation of gold particles, we use an adaptive threshold selection for each image. First the algorithm consists of performing a survey of segmentation results for different thresholds. Then, based on this survey, a threshold is selected such that it results in high detection rate combined with negligible false positive rate (FIG. 13C). Three segmentation regimes are shown at low threshold levels (th1 in FIGS. 13C and D) no objects are segmented. As the threshold is increased objects start to be detected. Due to its three dimensional nature, the core of the gold-labeled particle has higher density at the center (thus a lower gray scale value). As such, object median area increases with threshold, up to a point where some background pixels start to be included into the segmented results, at this point, the mean object area starts to decrease and that the number of objects starts to rise (second regime). It is within this regime that th2 threshold is selected (FIG. 13C). It is depicted in FIG. 13E that the adaptive threshold selection results in a satisfactory segmentation. Further increasing the threshold resulted in further decrease in object size and a fast increase in the number of segmented objects (third regime, th3 and its corresponding segmentation results (FIGS. 13C and F). Next, segmented object were filtered by their area, outliers where consider to be either smaller than the 2.5% or larger than the 97.5% quantiles or if having areas smaller than 9 nm2 or larger that 180 nn2; these values where obtained through inspection of the segmentation results in small inset dataset (n=10 images). Plaque density is then estimated in a ROI of about 500 nm2 (41×41 pixels) centered around each of the segmented and area-filtered gold particles. To obtain an unbiased metric the gold particle is removed after estimating the appropriate threshold, here again this process is performed for each ROI separately. This threshold is estimated based on set of four intensity profiles obtained by sampling the image across its main diagonal and along the pixel lines that cross the center of the ROI (a single such image profile is shown in FIG. 13G). Each intensity profile is processed to provide a single threshold estimate, then the median of these estimates id computed and further implemented. First the inverse of the profile is computed then threshold at 0.05% of the maximal value. The purpose of this step is to provide a sharp boundary between pixels along the image profile that belong to the gold particle and the ones that do not. Next, starting from the center of the intensity profile, the left and right first positions that equal zero are selected and their value along the image profile serves as single threshold estimate. By doing so, measuring other particles that might have been included in the current ROI is avoided. If such particles are present, they will also be removed when the median estimated threshold is applied to the ROI. Following gold particle removal, the total intensity is measured for the remaining pixels (‘included’ area in FIG. 13I) and divided by its corresponding area, thus providing an estimate of plaque density via its corresponding median grey level intensity. This algorithm was implemented in Matlab (Matworks, Inc.).

### REFERENCES

Amaducci and Tesco, 1994 Amaducci, L., and Tesco, G. (1994). Aging as a major risk for degenerative diseases of the central nervous system. Curr. Opin. Neurol. 7, 283-286.
Bar-On et al., 2006 Bar-On, P., Rockenstein, E., Adame, A., Ho, G., Hashimoto, M., and Masliah, E. (2006). Effects of the cholesterol-lowering compound methyl-beta-cyclodextrin in models of alpha-synucleinopathy. J. Neurochem. 98, 1032-1045.
Behrends et al., 2006 Behrends, C., Langer, C. A., Boteva, R., Bottcher, U. M., Stemp, M. J., Schaffar, G., Rao, B. V., Giese, A., Kretzschmar, H., and Siegers, K., et al. (2006). Chaperonin TRiC promotes the assembly of polyQ expansion proteins into nontoxic oligomers. Mol. Cell. 23, 887-897.
Blanchard et al., 2008 Blanchard, J., Martel, G., Guillou, J. L., Nogues, X., and Micheau, J. (2008). Impairment of spatial memory consolidation in APP(751SL) mice results in cue-guided response. Neurobiol. Aging 29, 1011-1021.
Bluher et al., 2003 Bluher, M., Kahn, B. B., and Kahn, C. R. (2003). Extended longevity in mice lacking the insulin receptor in adipose tissue. Science 299, 572-574.
Carro et al., 2002 Carro, E., Trejo, J. L., Gomez-Isla, T., LeRoith, D., and Torres-Aleman, I. (2002). Serum insulin-like growth factor I regulates brain amyloid-beta levels. Nat. Med. 8, 1390-1397.
Carro et al., 2006 Carro, E., Trejo, J. L., Spuch, C., Bohl, D., Heard, J. M., and Torres-Aleman, I. (2006). Blockade of the insulin-like growth factor I receptor in the choroid plexus originates Alzheimer's-like neuropathology in rodents: new cues into the human disease? Neurobiol. Aging 27, 1618-1631.
Cheng et al., 2007 Cheng, I. H., Scearce-Levie, K., Legleiter, J., Palop, J. J., Gerstein, H., Bien-Ly, N., Puolivali, J., Lesne, S., Ashe, K. H., and Muchowski, P. J., et al. (2007). Accelerating amyloid-beta fibrillization reduces oligomer levels and functional deficits in Alzheimer disease mouse models. J. Biol. Chem. 282, 23818-23828.
Cohen et al., 2006 Cohen, E., Bieschke, J., Perciavalle, R. M., Kelly, J. W., and Dillin, A. (2006). Opposing activities protect against age-onset proteotoxicity. Science 313, 1604-1610.
Cohen and Dillin, 2008 Cohen, E., and Dillin, A. (2008). The insulin paradox: aging, proteotoxicity and neurodegeneration. Nat. Rev. Neurosci. 9, 759-767.
Craft et al., 1998 Craft, S., Peskind, E., Schwartz, M. W., Schellenberg, G. D., Raskind, M., and Porte, D. (1998). Cerebrospinal fluid and plasma insulin levels in Alzheimer's disease: relationship to severity of dementia and apolipoprotein E genotype. Neurology 50, 164-168.
Farzan et al., 2000 Farzan, M., Schnitzler, C. E., Vasilieva, N., Leung, D., and Choe, H. (2000). BACE2, a beta-secretase homolog, cleaves at the beta site and within the amyloid-beta region of the amyloid-beta precursor protein. Proc. Natl. Acad. Sci. USA 97, 9712-9717.
Flachsbart et al., 2009 Flachsbart, F., Caliebe, A., Kleindorp, R., Blanche, H., von Eller-Eberstein, H., Nikolaus, S., Schreiber, S., and Nebel, A. (2009). Association of FOXO3A variation with human longevity confirmed in German centenarians. Proc. Natl. Acad. Sci. USA 106, 2700-2705.
Fukui et al., 2007 Fukui, H., Diaz, F., Garcia, S., and Moraes, C. T. (2007). Cytochrome c oxidase deficiency in neurons decreases both oxidative stress and amyloid formation in a mouse model of Alzheimer's disease. Proc. Natl. Acad. Sci. USA 104, 14163-14168.
Ghosh, 2008 Ghosh, H. S. (2008). The anti-aging, metabolism potential of SIRT1. Curr. Opin. Investig. Drugs 9, 1095-1102.
Glenner and Wong, 1984 Glenner, G. G., and Wong, C. W. (1984). Alzheimer's disease: initial report of the purification and characterization of a novel cerebrovascular amyloid protein. Biochem. Biophys. Res. Commun. 120, 885-890.
Haass and Selkoe, 2007 Haass, C., and Selkoe, D. J. (2007). Soluble protein oligomers in neurodegeneration: lessons from the Alzheimer's amyloid beta-peptide. Nat. Rev. 8, 101-112.
Hamos et al., 1989 Hamos, J. E., DeGennaro, L. J., and Drachman, D. A. (1989). Synaptic loss in Alzheimer's disease and other dementias. Neurology 39, 355-361.
Holzenberger et al., 2003 Holzenberger, M., Dupont, J., Ducos, B., Leneuve, P., Geloen, A., Even, P. C., Cervera, P., and Le Bouc, Y. (2003). IGF-1 receptor regulates lifespan and resistance to oxidative stress in mice. Nature 421, 182-187.
Jankowsky et al., 2004 Jankowsky, J. L., Fadale, D. J., Anderson, J., Xu, G. M., Gonzales, V., Jenkins, N. A., Copeland, N. G., Lee, M. K., Younkin, L. H., and Wagner, S. L., et al. (2004). Mutant presenilins specifically elevate the levels of the 42 residue beta-amyloid peptide in vivo: evidence for augmentation of a 42-specific gamma secretase. Hum. Mol. Genet. 13, 159-170.
Jankowsky et al., 2001 Jankowsky, J. L., Slunt, H. H., Ratovitski, T., Jenkins, N. A., Copeland, N. G., and Borchelt, D. R. (2001). Co-expression of multiple transgenes in mouse CNS: a comparison of strategies. Biomol. Eng. 17, 157-165.
Jensen et al., 2005 Jensen, M. T., Mottin, M. D., Cracchiolo, J. R., Leighty, R. E., and Arendash, G. W. (2005). Lifelong immunization with human beta-amyloid (1-42) protects Alzheimer's transgenic mice against cognitive impairment throughout aging. Neuroscience 130, 667-684.
Kenyon et al., 1993 Kenyon, C., Chang, J., Gensch, E., Rudner, A., and Tabtiang, R. (1993). A C. elegans mutant that lives twice as long as wild type. Nature 366, 461-464.
Kimura et al., 1997 Kimura, K. D., Tissenbaum, H. A., Liu, Y., and Ruvkun, G. (1997). daf-2, an insulin receptor-like gene that regulates longevity and diapause in Caenorhabditis elegans. Science 277, 942-946.
King and Arendash, 2002 King, D. L., and Arendash, G. W. (2002). Behavioral characterization of the Tg2576 transgenic model of Alzheimer's disease through 19 months. Physiol. Behav. 75, 627-642.
Lehtinen et al., 2006 Lehtinen, M. K., Yuan, Z., Boag, P. R., Yang, Y., Villen, J., Becker, E. B., extends life span. Cell 125, 987-1001. Abstract|Full Text|PDF (969 kb)|CrossRef|PubMed.
Lesne et al., 2006 Lesne, S., Koh, M. T., Kotilinek, L., Kayed, R., Glabe, C. G., Yang, A., Gallagher, M., and Ashe, K. H. (2006). A specific amyloid-beta protein assembly in the brain impairs memory. Nature 440, 352-357.
Link, 1995 Link, C. D. (1995). Expression of human beta-amyloid peptide in transgenic Caenorhabditis elegans. Proc. Natl. Acad. Sci. USA 92, 9368-9372.
Mancardi et al., 1983 Mancardi, G. L., Liwnicz, B. H., and Mandybur, T. I. (1983). Fibrous astrocytes in Alzheimer's disease and senile dementia of Alzheimer's type. Acta Neuropathol. 61, 76-80.
Masliah and Rockenstein, 2000 Masliah, E., and Rockenstein, E. (2000). Genetically altered transgenic models of Alzheimer's disease. J. Neural Transm. Suppl. 59, 175-183.
Morley et al., 2002 Morley, J. F., Brignull, H. R., Weyers, J. J., and Morimoto, R. I. (2002). The threshold for polyglutamine-expansion protein aggregation and cellular toxicity is dynamic and influenced by aging in Caenorhabditis elegans. Proc. Natl. Acad. Sci. USA 99, 10417-10422.
Nadjar et al., 2008 Nadjar, A., Berton, O., Guo, S., Leneuve, P., Dovero, S., Diguet, E., Tison, F., Zhao, B., Holzenberger, M., and Bezard, E. (2008). IGF-1 signaling reduces neuro-inflammatory response and sensitivity of neurons to MPTP. Neurobiol. Aging 30, 2021-2130.
Oberdoerffer et al., 2008 Oberdoerffer, P., Michan, S., McVay, M., Mostoslaysky, R., Vann, J., Park, S. K., Hartlerode, A., Stegmuller, J., Hafner, A., and Loerch, P., et al. (2008). SIRT1 redistribution on chromatin promotes genomic stability but alters gene expression during aging. Cell 135, 907-918.
Partridge and Bruning, 2008 Partridge, L., and Bruning, J. C. (2008). Forkhead transcription factors and ageing. Oncogene 27, 2351-2363.
Reiserer et al., 2007 Reiserer, R. S., Harrison, F. E., Syverud, D. C., and McDonald, M. P. (2007). Impaired spatial learning in the APPSwe+PSEN1DeltaE9 bigenic mouse model of Alzheimer's disease. Genes Brain Behav. 6, 54-65.
Scheff et al., 1990 Scheff, S. W., DeKosky, S. T., and Price, D. A. (1990). Quantitative assessment of cortical synaptic density in Alzheimer's disease. Neurobiol. Aging 11, 29-37.
Schriner et al., 2005 Schriner, S. E., Linford, N. J., Martin, G. M., Treuting, P., Ogburn, C. E., Emond, M., Coskun, P. E., Ladiges, W., Wolf, N., and Van Remmen, H., et al. (2005). Extension of murine life span by overexpression of catalase targeted to mitochondria. Science 308, 1909-1911.
Selkoe, 2004 Selkoe, D. J. (2004). Alzheimer disease: mechanistic understanding predicts novel therapies. Ann. Intern. Med. 140, 627-638.
Shankar et al., 2008 Shankar, G. M., Li, S., Mehta, T. H., Garcia-Munoz, A., Shepardson, N. E., Smith, I., Brett, F. M., Farrell, M. A., Rowan, M. J., and Lernere, C. A., et al. (2008). Amyloid-beta protein dimers isolated directly from Alzheimer's brains impair synaptic plasticity and memory. Nat. Med. 14, 837-842.
Shorter and Lindquist, 2004 Shorter, J., and Lindquist, S. (2004). Hsp104 catalyzes formation and elimination of self-replicating Sup35 prion conformers. Science 304, 1793-1797.
Suh et al., 2008 Suh, Y., Atzmon, G., Cho, M. O., Hwang, D., Liu, B., Leahy, D. J., Barzilai, N., and Cohen, P. (2008). Functionally significant insulin-like growth factor I receptor mutations in centenarians. Proc. Natl. Acad. Sci. USA 105, 3438-3442.
Tatar et al., 2001 Tatar, M., Kopelman, A., Epstein, D., Tu, M. P., Yin, C. M., and Garofalo, R. S. (2001). A mutant Drosophila insulin receptor homolog that extends life-span and impairs neuroendocrine function. Science 292, 107-110.
Westerheide et al., 2009 Westerheide, S. D., Anckar, J., Stevens, S. M., Sistonen, L., and Morimoto, R. I. (2009). Stress-inducible regulation of heat shock factor 1 by the deacetylase SIRT1. Science 323, 1063-1066.
Westerman et al., 2002 Westerman, M. A., Cooper-Blacketer, D., Mariash, A., Kotilinek, L., Kawarabayashi, T., Younkin, L. H., Carlson, G. A., Younkin, S. G., and Ashe, K. H. (2002). The relationship between Abeta and memory in the Tg2576 mouse model of Alzheimer's disease. J. Neurosci. 22, 1858-1867.
Willcox et al., 2008 Willcox, B. J., Donlon, T. A., He, Q., Chen, R., Grove, J. S., Yano, K., Masaki, K. H., Willcox, D. C., Rodriguez, B., and Curb, J. D. (2008). FOXO3A genotype is strongly associated with human longevity. Proc. Natl. Acad. Sci. USA 105, 13987-13992.
Wirths et al., 2008 Wirths, O., Breyhan, H., Marcello, A., Cotel, M. C., Bruck, W., and Bayer, T. A. (2008). Inflammatory changes are tightly associated with neurodegeneration in the brain and spinal cord of the APP/PS1KI mouse model of Alzheimer's disease. Neurobiol. Aging, doi:10.1016/j.neurobiolaging.2008.06.011|in press.
Wolfe et al., 1999 Wolfe, M. S., Xia, W., Ostaszewski, B. L., Diehl, T. S., Kimberly, W. T., and Selkoe, D. J. (1999). Two transmembrane aspartates in presenilin-1 required for presenilin endoproteolysis and gamma-secretase activity. Nature 398, 513-517.

### SUPPLEMENTAL REFERENCES

Chana et al. (2003). Biological Pyschiatry 53: 1086-98.
Masliah et al. (2000). Science 287: 1265-1269.
Mucke et al. (2000). J Neurosci 20: 4050-58.
Rockenstein et al. (2007). J Neurosci 27: 1981-99.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating a patient suffering from a gain of function disease, wherein the method comprises administering to said patient a therapeutically effective amount of an agent that reduces IGF-1 signaling in said patient, and wherein the gain of function disease is a neurodegenerative disease.

2. The method of claim 1 wherein the gain of function disease is Alzheimer's disease.

3. The method of claim 2 wherein the agent that reduces IGF-1 signaling inhibits the binding of a ligand to an IGF1 receptor (IGF-1R).

4. The method of claim 3 wherein the ligand is IGF-1.

5. The method of claim 3 wherein the agent is a receptor antagonist.

6. The method of claim 5 wherein the agent is selected from the group consisting of a small molecule and a protein.

7. The method of claim 6 wherein the protein is an anti-IGF-1R antibody.

8. The method of claim 3 wherein the agent is a soluble IGF-1R.

9. The method of claim 2 wherein the agent decreases the level of IGF-1 in the serum.

10. The method of claim 2 wherein the agent decreases the level of IGF-1 in the brain.

11. The method of claim 2 wherein the agent reduces the expression of IGF-1R.

12. The method of claim 11 wherein the agent is an antisense nucleic acid or an RNA interfering agent.

13. The method of claim 11 wherein the agent is a small molecule.

14. The method of claim 2 wherein the agent activates a FOXO transcription factor.

15. The method of claim 14 wherein the agent that activates a FOXO transcription factor is selected from the group consisting of an agent that increases deacetylation of the FOXO transcription factor, an agent that decreases phosphorylation of the FOXO transcription factor, an agent that promotes nuclear translocation of the FOXO transcription factor and an agent that increases binding to a FOXO transcriptional co-regulator.

16. The method of claim 2 wherein the agent inhibits the phosphorylation of IGF-1R.

17. A method of inducing Aβ hyper-aggregation in a patient in need thereof, wherein the method comprises administering to said patient a therapeutically effective amount of an agent that reduces IGF-1 signaling.

18. The method of claim 17 wherein the agent that reduces IGF-1 signaling inhibits the binding of a ligand to an IGF-1R.

19. The method of claim 18 wherein the agent is a receptor antagonist.

20. The method of claim 19 wherein the agent is selected from the group consisting of a small molecule and a protein.

21. The method of claim 20 wherein the protein is an anti-IGF-1R antibody.

22. The method of claim 21 wherein the agent is a soluble IGF-1R.

23. The method of claim 17 wherein the agent decreases the level of IGF-1 in the serum.

24. The method of claim 17 wherein the agent decreases the level of IGF-1 in the brain.

25. The method of claim 17 wherein the agent reduces the expression of IGF-1R.

26. The method of claim 25 wherein the agent is an antisense nucleic acid or an RNA interfering agent.

27. The method of claim 25 wherein the agent is a small molecule.

28. The method of claim 17 wherein the agent activates a FOXO transcription factor.

29. The method of claim 28 wherein the agent that activates a FOXO transcription factor is selected from the group consisting of an agent that increases deacetylation of the FOXO transcription factor, an agent that decreases phosphorylation of the FOXO transcription factor, an agent that promotes nuclear translocation of the FOXO transcription factor and an agent that increases binding to a FOXO transcriptional co-regulator.

30. The method of claim 17 wherein the agent inhibits the phosphorylation of IGF-1R.

31. A method of reducing Aβ proteotoxicity in a patient in need thereof, wherein the method comprises administering to said patient a therapeutically effective amount of an agent that reduces IGF-1 signaling.

32. The method of claim 31 wherein the agent that reduces IGF-1 signaling inhibits the binding of a ligand to an IGF-1R.

33. The method of claim 32 wherein the agent is a receptor antagonist.

34. The method of claim 33 wherein the agent is selected from the group consisting of a small molecule and a protein.

35. The method of claim 34 wherein the protein is an anti-IGF-1R antibody.

36. The method of claim 32 wherein the agent is a soluble IGF-1R.

37. The method of claim 31 wherein the agent decreases the level of IGF-1 in the serum.

38. The method of claim 31 wherein the agent decreases the level of IGF-1 in the brain.

39. The method of claim 32 wherein the agent reduces the expression of IGF-1R.

40. The method of claim 39 wherein the agent is an antisense nucleic acid or an RNA interfering agent.

41. The method of claim 40 wherein the agent is a small molecule.

42. The method of claim 40 wherein the agent activates a FOXO transcription factor.

43. The method of claim 42 wherein the agent that activates a FOXO transcription factor is selected from the group consisting of an agent that increases deacetylation of the FOXO transcription factor, an agent that decreases phosphorylation of the FOXO transcription factor, an agent that promotes nuclear translocation of the FOXO transcription factor and an agent that increases binding to a FOXO transcriptional co-regulator.

44. The method of claim 31 wherein the agent decreases the level of IGF-1 in the brain.

45. The method of claim 31 wherein the agent inhibits the phosphorylation of IGF-1R.
